(19) United States
(12) Patent Application Publication
(10) Pub. No.: US 2026/0217785 A1
(43) Pub. Date: Jul. 30, 2026

(54) INSULIN-FC FUSION PROTEINS AND METHODS OF USE TO TREAT CANCER

(71) Applicant: AKSTON BIOSCIENCES CORPORATION, Beverly, MA (US)

(72) Inventors: Todd C. ZION, Salem, MA (US); Thomas M. LANCASTER, Wenham, MA (US)

(21) Appl. No.: 19/143,692

(22) PCT Filed: Dec. 13, 2023
(86) PCT No.: PCT/US2023/083765

§ 371 Date: Jun. 26, 2025

Related U.S. Application Data
(60) Provisional application No. 63/477,576, filed on Dec. 29, 2022.

(51) Int. Cl. C07K 14/62; A61K 9/00; A61K 38/00; A61K 45/06; A61P 35/00; C12N 15/62

(52) U.S. Cl. CPC C07K 14/62; A61K 9/0019; A61K 45/06; A61P 35/00; C12N 15/62; A61K 38/00; C07K 2319/30

(57) ABSTRACT

The present disclosure relates to fusion proteins, e.g., insulin-Fc fusion proteins, compositions thereof, and their use to treat cancer cells and cancer tumors in subjects in need thereof. The fusion protein exhibits an anti-tumor effect on a cancer cell in the subject after administration, such as downregulation of insulin receptor, downregulation of insulin-like growth factor 1 receptor (IGF1R), decreased phosphorylated Akt, and a combination thereof. The disclosure relates to methods of downregulating insulin-like growth factor 1 receptor (IGF1R) in a subject in need thereof, as well as methods of inhibiting cancer cell metabolism, growth, and/or proliferation.

### CROSS-REFERENCE TO RELATED APPLICATIONS

The present application claims the priority benefit of U.S. Provisional patent application Ser. No. 63/477,576, filed Dec. 29, 2022, entitled INSULIN-FC FUSION PROTEINS AND METHODS OF USE TO TREAT CANCER, incorporated by reference in its entirety herein.

### SEQUENCE LISTING

The following application contains a sequence listing submitted electronically as a Standard ST.26 compliant XML file entitled “SequenceListing_ABC-048.xml,” created on Dec. 12, 2023, as 60,412 bytes in size, the contents of which are incorporated herein.

### TECHNICAL FIELD

The present disclosure relates to compositions of insulin-Fc fusion proteins and their use to treat cancer and cancer tumors.

### BACKGROUND

The following description of the background of the present technology is provided simply as an aid in understanding the present technology and is not admitted to describe or constitute prior art to the present technology.

Cancer is the second leading cause of death in the United States, with a projected population of 18 million cancer patients in 2020 and annual costs exceeding $170 billion, and accounting for almost 600,000 deaths annually. Surgery remains the best treatment available, but its applicability is often restricted to localized primary tumors and associated lymph nodes. Broad-based approaches such as radiation and chemotherapy have long been used together with surgery to improve disease control or to treat metastatic disease and are effective against many cancer types. However, neither approach can discriminate between rapidly dividing normal cells and cancerous cells, leading to adverse effects that often limit dosing to sub-efficacious levels, and subsets of tumor cells are either intrinsically resistant or can acquire resistance to these treatments through various mechanisms. These limitations motivate a shift in research focus to selectively target genetic and biochemical drivers of the disease.

### SUMMARY OF THE PRESENT TECHNOLOGY

In one embodiment, the present technology discloses a fusion protein comprising an insulin polypeptide and an Fc fragment, wherein the fusion protein comprises the sequence:

(SEQ ID NO: 18)
FVNQHLCGSDLVEALALVCGERGFFYTDPTGGGPRRGIVEQCCHS
ICSLYQLENYCNGGGGAGGGGDKTHTCPPCPAPELLGGPSVFLFP
PKPKDTLMISRTPEVTCVVVDVSHEDPEVKFNWYVDGVEVHNAKT
KPREEQYSSTYRVVSVLTVLHQDWLNGKEYKCKVSNKALPAPIEK
TISKAKGQPREPQVYTLPPSRDELTKNQVSLTCLVKGFYPSDIAV
EWESNGQPENNYKTTPPVLDSDGSFFLYSKLTVDKSRWQQGNVFS
CSVMHEALHNHYTQKSLSLSPG.

In one embodiment, the present technology discloses a fusion protein comprising an insulin polypeptide and an Fc fragment, wherein the fusion protein comprises the sequence:

(SEQ ID NO: 20)
FVNQHLCGSDLVEALALVCGERGFFYTDPTGGGPRRGIVEQCCHS
ICSLYQLENYCNGGGGAGGGGDKTHTCPPCPAPELLGGPSVFLFP
PKPKDTLMISRTPEVTCVVVDVSHEDPEVKFNWYVDGVEVHNAKT
KPREEQYQSTYRVVSVLTVLHQDWLNGKEYKCKVSNKALPAPIEK
TISKAKGQPREPQVYTLPPSRDELTKNQVSLTCLVKGFYPSDIAV
EWESNGQPENNYKTTPPVLDSDGSFFLYSKLTVDKSRWQQGNVFS
CSVMHEALHNHYTQKSLSLSPG.

In embodiments, the fusion protein comprises an insulin polypeptide and an Fc fragment, wherein the insulin polypeptide comprises the sequence:

(SEQ ID NO: 6)
FVNQHLCGSDLVEALALVCGERGFFYTDPTGGGPRRGIVEQCCHS
ICSLYQLENYCN

and wherein the fusion protein has a maximum insulin receptor binding IC₅₀ ratio relative to recombinant human insulin (RHI) of less than or equal to 50, less than or equal to 40, less than or equal to 30, or more preferably less than or equal to 20.

In some embodiments, the Fc fragment of the fusion protein comprises the sequence:

(SEQ ID NO: 32)
DKTHTCPPCPAPELLGGPSVFLFPPKPKDTLMISRTPEVTCVVVD
VSHEDPEVKFNWYVDGVEVHNAKTKPREEQYSSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G.

In some embodiments, the Fc fragment of the fusion protein comprises the sequence:

(SEQ ID NO: 33)
DKTHTCPPCPAPELLGGPSVFLFPPKPKDTLMISRTPEVTCVVVD
VSHEDPEVKFNWYVDGVEVHNAKTKPREEQYQSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G.

In embodiments, the insulin polypeptide and the Fc fragment are connected by a linker, wherein the linker comprises the sequence:

(SEQ ID NO: 11)
GGGGAGGGG.

In embodiments, the fusion protein has a maximum insulin receptor binding IC₅₀ ratio relative to recombinant human insulin (RHI) of less than or equal to 50, less than or equal to 40, less than or equal to 30, or more preferably less than or equal to 20.

Embodiments with lower IC₅₀ ratios demonstrate higher affinities for the insulin receptor than embodiments with higher IC₅₀ ratios. Insulin-Fc fusion proteins that are dimeric with respect to the insulin polypeptide (e.g. two moles of insulin polypeptide chain per mole of insulin-Fc homodimer, e.g. 2 moles of insulin analog B-chain and 2 moles of insulin analog A-chain per mole of insulin-Fc homodimer) with maximum insulin receptor binding IC₅₀ ratios relative to recombinant human insulin (RHI) of less than or equal to 50, less than or equal to 40, less than or equal to 30, or more preferably less than or equal to 20 demonstrate greater downregulation of insulin receptor (IR) compared to recombinant human insulin (RHI) (e.g. which has one mole of insulin polypeptide with respect to RHI, e.g. one mole of insulin B-chain and one mole of insulin A-chain per mole of RHI). Without wishing to be bound by any theory, it is hypothesized that insulin-Fc fusion proteins that (i) are dimeric with respect to the insulin polypeptide (e.g., two insulin polypeptide chains per insulin-Fc homodimer) and (ii) demonstrate maximum insulin receptor binding IC₅₀ ratios relative to recombinant human insulin (RHI) of less than or equal to 50, less than or equal to 40, less than or equal to 30, or more preferably less than or equal to 20, will demonstrate the ability to downregulate the insulin receptor (as shown in FIG. 4). Downregulation of insulin receptor is a key aspect to the efficacy of the preferred fusion proteins of the present technology. In embodiments, the insulin-Fc fusion proteins of the present technology have decreased Fc(gamma) receptor binding and decreased C1q binding, resulting in a long-term treatment option for certain cancers that does not generate anti-drug antibodies with repeated use. In particular, the insulin-Fc fusion proteins of the present technology are effective in reducing the size of tumors in which the IGF1 receptor (IFG1R), which is a transmembrane receptor found on the surface of cells, is overexpressed. In embodiments, the insulin-Fc fusion proteins of the present technology may be used with other anti-IGF1R therapies which may have developed resistance through alternate signaling pathways.

In embodiments, the nucleic acid (cDNA) encoding the fusion protein of SEQ ID NO: 18 comprises the following nucleic acid sequence:

(SEQ ID NO: 19)
ATGGAATGGAGCTGGGTCTTTCTCTTCTTCCTGTCAGTAACGACT
GGTGTCCACTCCTTCGTGAACCAGCACCTGTGCGGCTCCGACCTG
GTGGAAGCTCTGGCTCTCGTGTGCGGCGAGCGGGGCTTCTTCTAC
ACCGATCCCACTGGAGGCGGTCCACGCAGAGGCATCGTGGAACAG
TGCTGCCACTCCATCTGCTCCCTGTACCAGCTGGAAAACTACTGC
AATGGCGGAGGTGGTGCAGGAGGCGGTGGAGACAAAACTCACACA
TGCCCACCGTGCCCAGCACCTGAACTCCTGGGGGGACCGTCAGTC
TTCCTCTTCCCCCCAAAACCCAAGGACACCCTCATGATCTCCCGG
ACCCCTGAGGTCACATGCGTGGTGGTGGACGTGAGCCACGAAGAC
CCTGAGGTCAAGTTCAACTGGTACGTGGACGGCGTGGAGGTGCAT
AATGCCAAGACAAAGCCGCGGGAGGAGCAGTACAGCAGCACGTAC
CGTGTGGTCAGCGTCCTCACCGTCCTGCACCAGGACTGGCTGAAT
GGCAAGGAGTACAAGTGCAAGGTCTCCAACAAAGCCCTCCCAGCC
CCCATCGAGAAAACCATCTCCAAAGCCAAAGGGCAGCCCCGAGAA
CCACAGGTGTACACCCTGCCCCCATCCCGGGATGAGCTGACCAAG
AACCAGGTCAGCCTGACCTGCCTGGTCAAAGGCTTCTATCCCAGC
GACATCGCCGTGGAGTGGGAGAGCAATGGGCAGCCGGAGAACAAC
TACAAGACCACGCCTCCCGTGCTGGACTCCGACGGCTCCTTCTTC
CTCTACAGCAAGCTCACCGTGGACAAGAGCAGGTGGCAGCAGGGG
AACGTCTTCTCATGCTCCGTGATGCATGAGGCTCTGCACAACCAC
TACACGCAGAAGAGCCTCTCCCTGTCTCCGGGTTAG.

In embodiments, the nucleic acid (cDNA) encoding the fusion protein of SEQ ID NO: 20 comprises the following nucleic acid sequence:

(SEQ ID NO: 21)
ATGGAATGGAGCTGGGTCTTTCTCTTCTTCCTGTCAGTAACGACT
GGTGTCCACTCCTTCGTGAACCAGCACCTGTGCGGCTCCGACCTG
GTGGAAGCTCTGGCTCTCGTGTGCGGCGAGCGGGGCTTCTTCTAC
ACCGATCCCACTGGAGGCGGTCCACGCAGAGGCATCGTGGAACAG
TGCTGCCACTCCATCTGCTCCCTGTACCAGCTGGAAAACTACTGC
AATGGCGGAGGTGGTGCAGGAGGCGGTGGAGACAAAACTCACACA
TGCCCACCGTGCCCAGCACCTGAACTCCTGGGGGGACCGTCAGTC
TTCCTCTTCCCCCCAAAACCCAAGGACACCCTCATGATCTCCCGG
ACCCCTGAGGTCACATGCGTGGTGGTGGACGTGAGCCACGAAGAC
CCTGAGGTCAAGTTCAACTGGTACGTGGACGGCGTGGAGGTGCAT
AATGCCAAGACAAAGCCGCGGGAGGAGCAGTACCAGAGCACGTAC
CGTGTGGTCAGCGTCCTCACCGTCCTGCACCAGGACTGGCTGAAT
GGCAAGGAGTACAAGTGCAAGGTCTCCAACAAAGCCCTCCCAGCC
CCCATCGAGAAAACCATCTCCAAAGCCAAAGGGCAGCCCCGAGAA
CCACAGGTGTACACCCTGCCCCCATCCCGGGATGAGCTGACCAAG
AACCAGGTCAGCCTGACCTGCCTGGTCAAAGGCTTCTATCCCAGC
GACATCGCCGTGGAGTGGGAGAGCAATGGGCAGCCGGAGAACAAC
TACAAGACCACGCCTCCCGTGCTGGACTCCGACGGCTCCTTCTTC
CTCTACAGCAAGCTCACCGTGGACAAGAGCAGGTGGCAGCAGGGG
AACGTCTTCTCATGCTCCGTGATGCATGAGGCTCTGCACAACCAC
TACACGCAGAAGAGCCTCTCCCTGTCTCCGGGTTAG.

In embodiments, a cell may be engineered to express the fusion protein. The cell may be transfected with a nucleic acid encoding the fusion protein. In some examples, the cell is an HEK293 cell or a CHO cell.

In some embodiments, the fusion protein comprises a dimer of two identical monomers bound together via disulfide bonds, e.g., the fusion protein is a homodimer.

In some embodiments, the fusion protein comprises domains in the following orientation from N- to C-termini: (N-terminus)—insulin polypeptide—linker—Fc fragment—(C-terminus), and wherein the insulin polypeptide comprises domains in the following orientation from N- to C-termini: (N-terminus)—B-chain—C-peptide—A-chain-(C-terminus).

In one embodiment, the present technology discloses pharmaceutical compositions for inhibiting cancer cell metabolism, growth, and/or proliferation. In embodiments, pharmaceutical compositions comprising the insulin-Fc fusion proteins of the present technology demonstrate decreased Fc(gamma) receptor binding and decreased C1q binding, resulting in a long-term treatment option for cancer tumors that does not generate anti-drug antibodies with repeated use. In particular, pharmaceutical compositions comprising the insulin-Fc fusion proteins of the present technology are effective in reducing the size of cancer tumors in which the IFG1R is overexpressed. In embodiments, pharmaceutical compositions of the insulin-Fc fusion proteins of the present technology may be used with other anti-IGF1R therapies which may have developed resistance through alternate signaling pathways.

In embodiments, the pharmaceutical compositions comprise a fusion protein, for example an insulin-Fc fusion protein, dispersed in a pharmaceutically acceptable carrier (e.g. a buffer, e.g. a sodium phosphate buffer, e.g. a sodium phosphate and sodium chloride solution, e.g. a buffer solution optionally containing an additive, e.g. wherein the additive is polysorbate-20 or polysorbate-80) wherein the fusion protein comprises an insulin polypeptide and an Fc fragment connected by a linker, and where the maximum insulin receptor binding IC₅₀ ratio relative to recombinant human insulin (RHI) is less than or equal to 50, less than or equal to 40, less than or equal to 30, or more preferably less than or equal to 20. In embodiments, the pharmaceutical composition comprises a fusion protein comprising the insulin polypeptide of

(SEQ ID NO: 6)
FVNQHLCGSDLVEALALVCGERGFFYTDPTGGGPRRGIVEQCCHS
ICSLYQLENYCN.

In embodiments, the pharmaceutical composition comprises a fusion protein wherein the insulin polypeptide and the Fc fragment of the fusion protein are connected by a linker comprising the sequence GGGGAGGGG (SEQ ID NO: 11).

In embodiments, the pharmaceutical composition comprises a fusion protein dispersed in a pharmaceutically acceptable carrier, wherein the Fc fragment of the fusion protein comprises the sequence:

(SEQ ID NO: 32)
DKTHTCPPCPAPELLGGPSVFLFPPKPKDTLMISRTPEVTCVVVD
VSHEDPEVKFNWYVDGVEVHNAKTKPREEQYSSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G.

In embodiments, the pharmaceutical composition comprises a fusion protein dispersed in a pharmaceutically acceptable carrier, wherein the Fc fragment of the fusion protein comprises the sequence:

(SEQ ID NO: 33)
DKTHTCPPCPAPELLGGPSVFLFPPKPKDTLMISRTPEVTCVVVD
VSHEDPEVKFNWYVDGVEVHNAKTKPREEQYQSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G.

In one embodiment, the present technology discloses a pharmaceutical composition for inhibiting cancer cell metabolism, growth, and/or proliferation, wherein the pharmaceutical composition comprises a fusion protein dispersed in a pharmaceutically acceptable carrier, the fusion protein comprising an insulin polypeptide and an Fc fragment, wherein the insulin polypeptide and the Fc fragment are connected by a linker, and wherein the fusion protein comprises the sequence:

(SEQ ID NO: 18)
FVNQHLCGSDLVEALALVCGERGFFYTDPTGGGPRRGIVEQCCHS
ICSLYQLENYCNGGGGAGGGGDKTHTCPPCPAPELLGGPSVFLFP
PKPKDTLMISRTPEVTCVVVDVSHEDPEVKFNWYVDGVEVHNAKT
KPREEQYSSTYRVVSVLTVLHQDWLNGKEYKCKVSNKALPAPIEK
TISKAKGQPREPQVYTLPPSRDELTKNQVSLTCLVKGFYPSDIAV
EWESNGQPENNYKTTPPVLDSDGSFFLYSKLTVDKSRWQQGNVFS
CSVMHEALHNHYTQKSLSLSPG.

In one embodiment, the present technology discloses a pharmaceutical composition for inhibiting cancer cell metabolism, growth, and/or proliferation, wherein the pharmaceutical composition comprises a fusion protein dispersed in a pharmaceutically acceptable carrier, the fusion protein comprising an insulin polypeptide and an Fc fragment, wherein the insulin polypeptide and the Fc fragment are connected by a linker, and wherein the fusion protein comprises the sequence:

(SEQ ID NO: 20)
FVNQHLCGSDLVEALALVCGERGFFYTDPTGGGPRRGIVEQCCHS
ICSLYQLENYCNGGGGAGGGGDKTHTCPPCPAPELLGGPSVFLFP
PKPKDTLMISRTPEVTCVVVDVSHEDPEVKFNWYVDGVEVHNAKT
KPREEQYQSTYRVVSVLTVLHQDWLNGKEYKCKVSNKALPAPIEK
TISKAKGQPREPQVYTLPPSRDELTKNQVSLTCLVKGFYPSDIAV
EWESNGQPENNYKTTPPVLDSDGSFFLYSKLTVDKSRWQQGNVFS
CSVMHEALHNHYTQKSLSLSPG.

In embodiments, the pharmaceutical composition for use in treating cancer, wherein the pharmaceutical composition comprises a fusion protein dispersed in a pharmaceutically acceptable carrier, and wherein the pharmaceutical composition exhibits an anti-tumor effect on a cancer cell, wherein the anti-tumor effect is selected from the group consisting of downregulation of insulin receptor, downregulation of insulin-like growth factor 1 receptor (IGF1R), decreased phosphorylated Akt, or a combination thereof, as compared to an untreated control cancer cell.

In embodiments, the present technology discloses a method of inhibiting cancer cell metabolism, growth, and/or proliferation or cancer tumor growth, the method comprising administering an effective amount of a fusion protein or administering a pharmaceutical composition comprising an effective amount of a fusion protein dispersed in a pharmaceutically acceptable carrier, to a subject in need thereof. That is, the fusion protein inhibits cancer cell metabolism, growth, and/or proliferation or cancer tumor growth in the subject after the administration of the fusion protein. In embodiments, the fusion protein or the pharmaceutical composition comprising an effective amount of a fusion protein dispersed in a pharmaceutically acceptable carrier is administered to said subject under fasted conditions. In embodiments, the subject has been diagnosed with a cancer selected from the group consisting of breast cancer, colorectal cancer, and melanoma.

In examples, the fusion proteins of the present technology are effective in downregulating insulin-like growth factor 1 receptor (IGF1R) in a subject in need thereof, wherein IGR1R is downregulated in said subject after said administration, wherein said subject has a reduction in tumor volume as compared to an untreated control tumor.

In examples, gene amplification of insulin growth factor 1 receptor (IGF1R), which encodes insulin-like growth factor 1 receptor, is a frequent event across numerous cancer types. The IGF axis, of which IGF1R is a surface cell receptor, is involved in cancer development, metastasis and cancer therapeutic resistance. Among the molecules of the IGF axis, IGF1R is recognized as a promising target of cancer therapy because of its frequent amplification and overexpression in various cancers and its established functions in transmitting tumorigenic signals, in addition to its role as the primary receptor of the insulin growth factors.

Overexpression of IGF1R has been reported in multiple human cancer lineages and was associated with poor prognosis of cancer patients. For example, in prostate cancer, protein levels of IGF1R have been shown to be significantly higher in malignant epithelia compared with benign glands from the same biopsies. In examples of head and neck squamous cell cancer, patients with high IGF1R levels have been shown to have significantly reduced overall survival and disease-specific survival. Studies have shown that at least 50% of breast cancer cases were found to express activated IGF1R. (Yuan J, Yin Z, Tao K, Wang G, Gao J. Function of insulin-like growth factor 1 receptor in cancer resistance to chemotherapy. Oncol Lett. 2018 January; 15(1):41-47. doi: 10.3892/ol.2017.7276. Epub 2017 October 26. PMID: 29285186; PMCID: PMC57386963@PAlfaro-Arnedo, F., López, I. P., Piñeiro-Hermida, S. et al. IGF1R acts as a cancer-promoting factor in the tumor microenvironment facilitating lung metastasis implantation and progression. Oncogene 41, 3625-3639 (2022); Amutha P, Rajkumar T. Role of Insulin-like Growth Factor, Insulin-like Growth Factor Receptors, and Insulin-like Growth Factor-binding Proteins in Ovarian Cancer. Indian J Med Paediatr Oncol. 2017 April-June; 38(2):198-206; Sun Y, Sun X, Shen B. Molecular Imaging of IGF-1R in Cancer. Mol Imaging. 2017 January-December; 16:1536012117736648. doi: 10.11771536012117736648. PMID: 29169312; PMCID: PMC5703088).

Examples of the most prevalent cancers with overexpression of IG1FR are uterine corpus endometrial carcinoma, stomach adenocarcinoma, skin cutaneous melanoma, sarcoma, breast invasive carcinoma, ovarian serous cystadenocarcinoma, ovarian serous cystadenocarcinoma, adrenocortical carcinoma, esophageal adenocarcinoma, liver hepatocellular carcinoma, lung squamous cell carcinoma, bladder urothelial carcinoma, cervical squamous cell carcinoma, colorectal adenocarcinoma, lung adenocarcinoma, and pancreatic adenocarcinoma (Panpan Wang, Victor C Y. Mak, Lydia W T. Cheung, Drugging IGF-1R in cancer: New insights and emerging opportunities, Genes & Diseases, 2022, ISSN 2352-3042.) The insulin-Fc fusion proteins of the present technology are expected to reduce tumor volumes in types of cancers with overexpression of IG1FR.

In some embodiments, the subject may exhibit a reduction in tumor volume of at least 40% after the administration of the insulin-Fc fusion protein, compared to a fasted untreated control. In some embodiments, the subject may exhibit a reduction in tumor volume of at least 30% after the administration of the insulin-Fc fusion protein, compared to an unfasted untreated control. In embodiments, the primary or secondary cancer therapy are selected from the group consisting of chemotherapy agents, tamoxifen agonists, or antibodies against the IGF1R.

In embodiments, the present technology discloses a method of inhibiting cancer cell metabolism, growth, and/or proliferation or cancer tumor growth, wherein the method comprises administering an effective amount of a fusion protein or of a pharmaceutical composition comprising the fusion protein dispersed in a pharmaceutically acceptable carrier exhibits an anti-tumor effect on a cancer cell, wherein the anti-tumor effect is selected from the group consisting of downregulation of insulin receptor, downregulation of insulin-like growth factor 1 receptor (IGF1R), decreased phosphorylated Akt, or a combination thereof, as compared to an untreated control cancer cell.

In embodiments, the fusion protein or a pharmaceutical composition comprising the fusion protein dispersed in a pharmaceutically acceptable carrier is administered by intravenous injection, by subcutaneous injection, or by intratumoral injection. In embodiments, the fusion protein or a pharmaceutical composition comprising the fusion protein dispersed in a pharmaceutically acceptable carrier is administered as a bolus, as an infusion, or as an intravenous push. In embodiments, the fusion protein or a pharmaceutical composition comprising the fusion protein dispersed in a pharmaceutically acceptable carrier is administered through syringe injection, or using a pump, a pen, a needle, or an indwelling catheter.

The present invention is also directed to the use of the fusion protein of the invention for the manufacture of a medicament for the treatment of cancer, preferably inhibiting cancer cell metabolism, growth, and/or proliferation.

Furthermore, said fusion protein may exhibit an anti-tumor effect on a cancer cell in said subject after administration, said anti-tumor effect being selected from the group consisting of downregulation of insulin receptor, downregulation of insulin-like growth factor 1 receptor (IGF1R), decreased phosphorylated Akt, and a combination thereof, as compared to an untreated control cancer cell.

Ideally, the fusion protein is for use via intravenous, subcutaneous, or intratumoral injection and/or may be administered as a bolus, infusion, or an intravenous push. Preferably, the fusion protein may be administered through syringe injection, pump, pen, needle, or indwelling catheter. Additionally, the fusion protein may be co-administered with a primary or secondary cancer therapy selected from the group consisting of chemotherapy agents, tamoxifen agonists, or antibodies against the IGF1R.

According to a preferred embodiment, the fusion may be administered to said subject at a dose of from about 150 to about 1,500 micrograms per kilogram of body weight per day. Additionally, the fusion protein may be administered to a subject under fasted or unfasted conditions.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including Definitions, will control. In addition, the materials, methods, and examples are illustrative only and are not intended to be limiting. Other features and advantages of the disclosure will be apparent from the following Detailed Description, Examples, and Claims.

### BRIEF DESCRIPTION OF THE DRAWINGS

FIG. 1 shows a schematic representation of an exemplary insulin-Fc fusion protein homodimer;

FIG. 2 illustrates the “full aa sequence” of a fusion protein (SEQ ID NO: 18) including the leader sequence of SEQ ID NO: 14, that is the cDNA sequence of SEQ ID NO: 15 and the cDNA sequence of SEQ ID NO: 19 corresponding to the amino acid sequence of SEQ ID NO: 14 and SEQ ID NO: 18, respectively;

FIG. 3 illustrates the “full aa sequence” of a fusion protein (SEQ ID NO: 20) including the leader sequence of SEQ ID NO: 14, that is the cDNA sequence of SEQ ID NO: 15 and the cDNA sequence of SEQ ID NO: 21 corresponding to the amino acid sequence of SEQ ID NO: 14 and SEQ ID NO: 20, respectively;

FIG. 4 shows Western Blot images of HCT-116 bearing nude mice treated in vitro with medium only, RHI, SEQ ID NO: 1 or SEQ ID NO: 47 at concentrations between 0.05 and 500 nM;

FIG. 5 shows the percent fasting blood glucose level for SEQ ID NO: 1 and porcine insulin NPH vehicle;

FIG. 6 shows tumor volume ratios in HCT-116 bearing nude mice for SEQ ID NO: 1;

FIG. 7 shows tumor volume ratios in WM266.4 bearing nude mice for SEQ ID NO: 1;

FIG. 8 illustrates a side-by-side sequence comparison of SEQ ID NO: 1 and SEQ ID NO: 47. “*” represents complete homology across all sequences at a given sequence position, while “:”, “.” or spaces refer to conservative, moderate, or very different amino acid mutations across the sequences at a given sequence position, respectively;

FIG. 9 illustrates a side-by-side sequence comparison of SEQ ID NO: 1 and SEQ ID NO: 16. “*” represents complete homology across all sequences at a given sequence position, while “:”, “.” or spaces refer to conservative, moderate, or very different amino acid mutations across the sequences at a given sequence position, respectively;

FIG. 10 illustrates a side-by-side sequence comparison of SEQ ID NO: 1, SEQ ID NO: 22, SEQ ID NO: 24 and SEQ ID NO: 26. “*” represents complete homology across all sequences at a given sequence position, while “:”, “.” or spaces refer to conservative, moderate, or very different amino acid mutations across the sequences at a given sequence position, respectively;

FIG. 11 illustrates a side-by-side sequence comparison of SEQ ID NO: 1, SEQ ID NO: 28 and SEQ ID NO: 30. “*” represents complete homology across all sequences at a given sequence position, while “:”, “.” or spaces refer to conservative, moderate, or very different amino acid mutations across the sequences at a given sequence position, respectively;

FIG. 12 illustrates a side-by-side sequence comparison of SEQ ID NO: 1, SEQ ID NO: 18 and SEQ ID NO: 20. “*” represents complete homology across all sequences at a given sequence position, while “:”, “.” or spaces refer to conservative, moderate, or very different amino acid mutations across the sequences at a given sequence position, respectively.

### DETAILED DESCRIPTION

The present disclosure relates to compositions of fusion proteins, e.g., insulin-Fc fusion proteins, and their use to treat cancer tumors.

### Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

As used herein, an amount of a molecule, compound, conjugate, or substance effective to treat a disorder (e.g., a disorder described herein), “therapeutically effective amount,” or “effective amount” refers to an amount of the molecule, compound, conjugate, or substance which is effective, upon single or multiple dose administration(s) to a subject, in treating a subject, or in curing, alleviating, relieving or improving a subject with a disorder (e.g., a disorder described herein) beyond that expected in the absence of such treatment.

As used herein, the term “analog” refers to a compound or conjugate (e.g., a compound or conjugate as described herein, e.g., insulin) having a chemical structure similar to that of another compound or conjugate but differing from it in at least one aspect.

As used herein, the term “antibody” or “antibody molecule” refers to an immunoglobulin molecule (Ig), immunologically active portions of an immunoglobulin (Ig) molecule, i.e., a molecule that contains an antigen binding site that specifically binds, e.g., immunoreacts with, an antigen. As used herein, the term “antibody domain” refers to a variable or constant region of an immunoglobulin. As used herein, the term “antibody domain” refers to a variable or constant region of an immunoglobulin. It is documented in the art that antibodies comprise several classes, for example IgA, IgM, or IgG in the case of mammals (e.g., humans). Classes of immunoglobulins can be further classified into different isotypes such as IgGA, IgGB, IgGC, and IgGD for canines, and IgG1, IgG2, IgG3, and IgG4 for humans. Those skilled in the art will recognize that immunoglobulin isotypes of a given immunoglobulin class will comprise different amino acid sequences, structures, and functional properties from one another (e.g., different binding affinities to Fc(gamma) receptors). “Specifically binds” or “immunoreacts with” means that the antibody reacts with one or more antigenic determinants of the desired antigen and has a lower affinity for other polypeptides, e.g., does not react with other polypeptides.

As used herein the terms “insulin-Fc fusion protein” or “insulin-Fc protein” or “fusion protein” or “insulin-Fc fusion homodimer” refer to a protein comprising an insulin protein and an Fc fragment.

As used herein, the term “bioactivity,” “activity,” “biological activity,” “potency,” “bioactive potency,” or “biological potency” refers to the extent to which an insulin-Fc fusion protein activates the IR and/or exerts a reduction in blood glucose levels in a target subject. As used herein, “in vitro activity” or “IR activity” refers to the affinity with which an insulin-Fc fusion protein binds to the IR and is typically measured by the concentration at which an insulin-Fc fusion protein displaces half of an insulin reference standard from the IR in a competitive binding assay (i.e., IC₅₀). As used herein, “in vivo activity” refers to the extent and duration of reduction in a target subject's fasting blood glucose level after administration of an insulin-Fc fusion protein.

It is noted that fusion proteins of the current technology exhibit weaker binding to the insulin receptor than RHI, as demonstrated by an IC₅₀ ratio of greater than 1. The term “strong binding to the insulin receptor” indicates a relative comparison of the insulin receptor binding ratio to RHI between different insulin-Fc fusion protein compounds. For example, an insulin-Fc fusion protein (referred to as ‘insulin-Fc fusion protein A’) with an IC₅₀ ratio of 20 means that the insulin-Fc fusion protein binds to the insulin receptor 20 times weaker than recombinant human insulin. However, a different insulin-Fc fusion protein with an IC₅₀ ratio of greater than 100 (referred to as ‘insulin-Fc fusion protein A’) binds the insulin receptor much less strongly than referred to as ‘insulin-Fc fusion protein A’. In this case, we may state that ‘insulin-Fc fusion protein A exhibits strong binding to the insulin receptor’ or that ‘insulin-Fc fusion protein A exhibits stronger binding to the insulin receptor than a reference insulin-Fc fusion protein’, where the reference insulin-Fc fusion protein may be SEQ ID NO: 47 which has an IC₅₀ ratio of 142. Alternatively, we may state that ‘insulin-Fc fusion protein A exhibits a low IC₅₀ ratio to RHI’. Such expressions may be interchangeable used to express that the exemplary insulin-Fc fusion proteins of the present technology exhibit relatively strong binding to the insulin receptor compared to other insulin-Fc fusion proteins.

As used herein, the term “biosynthesis,” “recombinant synthesis,” or “recombinantly made” refers to the process by which an insulin-Fc fusion protein is expressed within a host cell by transfecting the cell with a nucleic acid molecule (e.g., vector) encoding the insulin-Fc fusion protein (e.g., where the entire insulin-Fc fusion protein is encoded by a single nucleic acid molecule). Exemplary host cells include mammalian cells, e.g., HEK293 cells or CHO cells. The cells can be cultured using standard methods in the art and the expressed insulin-Fc fusion protein may be harvested and purified from the cell culture using standard methods in the art.

As used herein, the term “cell surface receptor” refers to a molecule such as a protein, generally found on the external surface of the membrane of a cell and which interacts with soluble molecules, e.g., molecules that circulate in the blood supply. In some embodiments, a cell surface receptor may include a hormone receptor (e.g., an insulin hormone receptor or insulin receptor (IR)) or an Fc receptor which binds to an Fc fragment or the Fc region of an antibody (e.g., an Fc(gamma) receptor, for example Fc(gamma)RI, or an Fc neonatal receptor, for example FcRn). As used herein, “in vitro activity” or “Fc(gamma) receptor activity” or “Fc(gamma) receptor binding” or “FcRn receptor activity” or “FcRn binding” refers to the affinity with which an insulin-Fc fusion protein binds to the Fc receptor (e.g. Fc(gamma) receptor or FcRn receptor) and is typically measured by the concentration of an insulin-Fc fusion protein that causes the insulin-Fc fusion protein to reach half of its maximum binding (i.e., EC50 value) as measured on an assay (e.g., an enzyme-linked immunosorbent assay (ELISA) assay) using OD 450 nm values as measured on a microplate reader. Alternatively, the affinity with which an insulin-Fc fusion protein binds to the Fc receptor (e.g., Fc(gamma) receptor or FcRn receptor) is measured by the OD 450 nm value obtained on a microplate reader in an enzyme-linked immunosorbent assay (ELISA) assay at a given concentration of the insulin-Fc fusion protein.

As used herein, the term “C1q” or “complement component 1q” means a protein complex involved in the complement system, which is part of the innate immune system. C1q together with C1r and C1s form the C1 complex. C1q plays a role in involved in specific antigen presentation by dendritic cells to T cells and B cells.

As used herein, the term “fasting blood glucose level” or “FBGL” refers to the average blood glucose level in a target subject at the end of a period during which no food is administered and just prior to the time at which an insulin-Fc fusion protein is administered. As used herein, the term “percent fasting blood glucose level,” “% fasting blood glucose level,” or “% FBGL” refers to the ratio of a given blood glucose level to the fasting blood glucose level multiplied by 100.

As used herein, the term “immunogenic” or “immunogenicity” refers to the capacity for a given molecule (e.g., an insulin-Fc fusion protein of the present invention) to provoke the immune system of a target subject such that after repeated administrations of the molecule, the subject develops antibodies capable of specifically binding the molecule (i.e., anti-drug antibodies). As used herein, the terms “neutralizing,” “neutralizing antibodies”, or “neutralizing anti-drug antibodies” refer to the capacity for antibodies to interfere with the compound's biological activity in the target subject. As used herein, the term “immunogenic epitopes,” ‘immunogenic hot spots,” or “hot spots” refers to the mutations or epitopes of a given molecule (e.g., an insulin-Fc fusion protein of the present invention) that are responsible for moderate or strong binding of the anti-drug antibodies.

As used herein, the term “insulin reference standard” is any one of: (i) a naturally occurring insulin from a mammal (e.g., a human); (ii) an insulin polypeptide that does not comprise an Fc fragment; or (iii) a standard of care insulin (e.g., a commercially available insulin).

As used herein, the term “monomer” refers to a protein or a fusion protein comprising a single polypeptide. In embodiments, the “monomer” is a protein or a fusion protein, e.g., a single polypeptide, comprising an insulin polypeptide and an Fc fragment polypeptide, wherein the insulin and Fc fragment polypeptides are joined by peptide bonds to form the single polypeptide. In embodiments, the monomer is encoded by a single nucleic acid molecule.

As used herein, “N-terminus” refers to the start of a protein or polypeptide that is initiated by an amino acid containing a free amine group that is the alpha-amino group of the amino acid (e.g., the free amino that is covalently linked to one carbon atom that is located adjacent to a second carbon atom, wherein the second carbon atom is part of the carbonyl group of the amino acid). As used herein, “C-terminus” refers to the end of a protein or polypeptide that is terminated by an amino acid containing a carboxylic acid group, wherein the carbon atom of the carboxylic acid group is located adjacent to the alpha-amino group of the amino acid.

As used herein, “pharmacodynamics” or “PD” generally refers to the biological effects of an insulin-Fc fusion protein in a subject. Specifically, herein the PD refers to the measure of the reduction in fasting blood glucose level over time in a subject after the administration of an insulin-Fc fusion protein.

As used herein, “pharmacokinetics” or “PK” generally refers to the characteristic interactions of an insulin-Fc fusion protein and the body of the subject in terms of its absorption, distribution, metabolism, and excretion. Specifically, herein the PK refers to the concentration of an insulin-Fc fusion protein in the blood or serum of a subject at a given time after the administration of the insulin-Fc fusion protein. As used herein, “half-life” refers to the time taken for the concentration of insulin-Fc fusion protein in the blood or serum of a subject to reach half of its original value as calculated from a first order exponential decay model for drug elimination. Insulin-Fc fusion proteins with greater “half-life” values demonstrate greater duration of action in the target subject.

The terms “sequence identity” “sequence homology” “homology” or “identical” in amino acid or nucleotide sequences as used herein describes that the same nucleotides or amino acid residues are found within the variant and reference sequences when a specified, contiguous segment of the nucleotide sequence or amino acid sequence of the variant is aligned and compared to the nucleotide sequence or amino acid sequence of the reference sequence. Methods for sequence alignment and for determining identity between sequences are known in the art, including the use of Clustal Omega, which organizes, aligns, and compares sequences for similarity, wherein the software highlights each sequence position and compares across all sequences at that position and assigns one of the following scores: an “*” (asterisk) for sequence positions which have a single, fully conserved residue, a “:” (colon) indicates conservation between groups of strongly similar properties with scoring greater than 0.5 in the Gonnet PAM 250 matrix, and a “.” (period) indicates conservation between groups of weakly similar properties with scoring less than or equal to 0.5 in the Gonnet PAM 250 matrix, a “-” (dash) indicates a sequence gap, meaning that no local homology exists within a particular set of comparisons within a certain range of the sequences, and an empty space “” indicates little or no sequence homology for that particular position across the compared sequences. See, for example Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5: Suppl. 3 (National Biomedical Research Foundation, Washington, D.C.)). With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. In some embodiments, the contiguous segment used for comparison to the reference nucleotide sequence or reference amino acid sequence will comprise at least 6, 10, 15, or 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are known in the art.

In embodiments, the determination of percent identity or “homology” between two sequences is accomplished using a mathematical algorithm. For example, the percent identity of an amino acid sequence is determined using the Smith-Waterman homology search algorithm using an affine 6 gap search with a gap open penalty of 12 and a gap extension penalty of 2, BLOSUM matrix 62. The Smith-Waterman homology search algorithm is described in Smith and Waterman (1981) Adv. Appl. Math 2:482-489, herein incorporated by reference. In embodiments, the percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

As used herein, the term “homology” is used to compare two or more proteins by locating common structural characteristics and common spatial distribution of, for instance, beta strands, helices, and folds. Accordingly, homologous protein structures are defined by spatial analyses. Measuring structural homology involves computing the geometric-topological features of a space. One approach used to generate and analyze three-dimensional (3D) protein structures is homology modeling (also called comparative modeling or knowledge-based modeling) which works by finding similar sequences on the basis of the fact that 3D similarity reflects 2D similarity. Homologous structures do not imply sequence similarity as a necessary condition.

As used herein, the terms “subject” and “patient” are intended to include humans having a disease or a disorder, e.g., a cancerous tumor, diabetes or another disease or disorder described herein, or normal subjects.

As used herein, the term “titer” or “yield” refers to the amount of a fusion protein product (e.g., an insulin-Fc fusion protein described herein) resulting from the biosynthesis (e.g., in a mammalian cell, e.g., in a HEK293 cell or CHO cell) per volume of the cell culture. The amount of product may be determined at any step of the production process (e.g., before or after purification), but the yield or titer is always stated per volume of the original cell culture. As used herein, the term “product yield” or “total protein yield” refers to the total amount of insulin-Fc fusion protein expressed by cells and purified via at least one affinity chromatography step (e.g., Protein A or Protein G) and includes monomers of insulin-Fc fusion protein, homodimers of insulin-Fc fusion protein, and higher-order molecular aggregates of homodimers of insulin-Fc fusion protein. As used herein, the term “percent homodimer” or “% homodimer” refers to the proportion of a fusion protein product (e.g., an insulin-Fc fusion protein described herein) that is the desired homodimer. As used herein, the term “homodimer titer” refers to the product of the % homodimer and the total protein yield after Protein A purification step reported per volume of the cell culture.

As used herein, the terms “treat” or “treating” a subject having a disease or a disorder refer to subjecting the subject to a regimen, for example the administration of a fusion protein, such as a fusion protein described herein, such that at least one symptom of the disease or disorder is cured, healed, alleviated, relieved, altered, remedied, ameliorated, or improved. Treating includes administering an amount effective to alleviate, relieve, alter, remedy, ameliorate, improve, or affect the disease or disorder, or the symptoms of the disease or disorder. The treatment may inhibit deterioration or worsening of a symptom of a disease or disorder.

### Fusion Protein Components and Structure

The present disclosure relates to a composition of a fusion protein (i.e., an insulin-Fc fusion protein) comprising an insulin polypeptide linked either directly or via a peptide linker to a species-specific Fc fragment, and its use to treat cancer in mammals. As used herein, the terms “fusion protein” and “insulin-Fc fusion protein” refer to a protein comprising more than one part, for example from different sources (different proteins, polypeptides, cells, etc.), that are covalently linked through peptide bonds. Insulin-Fc fusion proteins may be covalently linked by (i) connecting the genes that encode for each part into a single nucleic acid molecule and (ii) expressing in a host cell (e.g., HEK or CHO) the protein for which the nucleic acid molecule encodes as follows: (N-terminus)—insulin polypeptide—linker—Fc fragment—(C-terminus). The fully recombinant synthesis approach is preferred over methods in which the insulin polypeptide and Fc fragments are synthesized separately and then chemically conjugated. The chemical conjugation step and subsequent purification process increase the manufacturing complexity, reduce product yield, and increase cost.

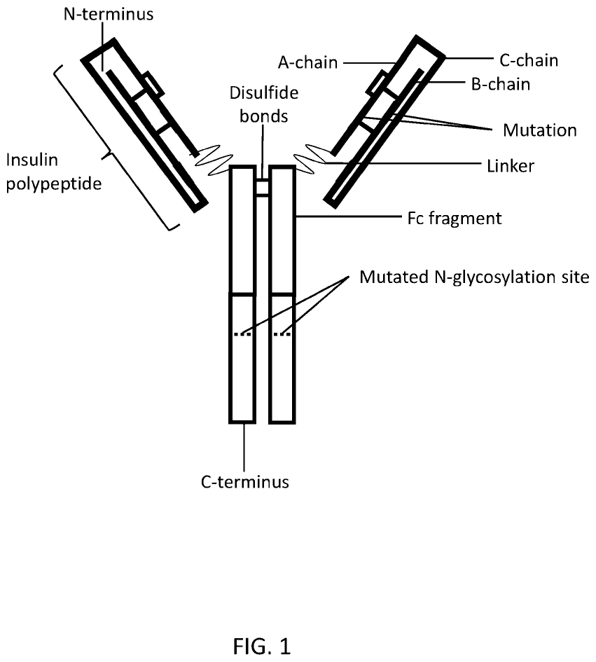

As used herein, the term “dimer” refers to a protein or a fusion protein comprising two polypeptides linked covalently. In embodiments, two identical polypeptides are linked covalently (e.g., via disulfide bonds) forming a “homodimer” (diagrammatically represented in FIG. 1). Disulfide bonds are shown in FIG. 1; the total number of disulfide bonds in actuality may be greater or less than the number shown in FIG. 1. In embodiments, the homodimer is encoded by a single nucleic acid molecule, wherein the homodimer is made recombinantly inside a cell by first forming insulin-Fc fusion protein monomers and by then assembling two identical insulin-Fc fusion protein monomers into the homodimer upon further processing inside the cell.

As used herein, the terms “multimer,” “multimeric,” or “multimeric state” refer to non-covalent, associated forms of Fc fusion protein dimers that may be in equilibrium with Fc fusion protein dimers or may act as permanently aggregated versions of Fc fusion protein dimers (e.g., dimers of Fc fusion protein homodimers, trimers of Fc fusion protein homodimers, tetramers of Fc fusion protein homodimers, or higher order aggregates containing five or more Fc fusion protein homodimers). It may be expected that multimeric forms of Fc fusion proteins may have different physical, stability, or pharmacologic activities from that of the insulin-Fc fusion protein homodimers.

### Insulin Polypeptide

In embodiments, the insulin-Fc fusion proteins described herein comprise an insulin polypeptide, e.g., an insulin or insulin analog. Insulin is a peptide hormone produced by β-cells in islets of Langerhans within the pancreas. Insulin functions by regulating the absorption of glucose from the blood. Upon a stimulus, such as increased protein and glucose levels, insulin is released from β-cells and binds to the insulin receptor, initiating a signal cascade that affects many aspects of mammalian metabolism. Disruption of this process is directly related to several diseases, notably diabetes, insulinoma, insulin resistance, metabolic syndromes, and polycystic ovary syndrome.

Insulin analogs of the present disclosure may be related to the structure of insulin yet contain one or more modifications. In some embodiments, the insulin analog comprises at least one amino acid substitution, deletion, addition, or chemical modification relative to insulin, which may impact a particular feature or characteristic of the insulin-Fc fusion protein configuration. For example, the modifications or alterations described herein may impact the structure, stability, pH sensitivity, bioactivity, or binding affinity of the insulin-Fc fusion protein configuration to a cell surface receptor (e.g., an insulin hormone receptor) relative to a reference standard.

The amino acid sequence of insulin is strongly conserved throughout evolution, particularly in vertebrates. For example, native canine and porcine insulins differ by only one amino acid from human insulin, native bovine insulin differs by only three amino acids from human insulin, and native feline insulin differs by just four amino acids from human insulin. As used herein, the terms “B-chain or B-chain analog”, “C-peptide” or “C-chain”, and “A-chain or A-chain analog” refer to the peptide segments of an insulin polypeptide as illustrated in FIG. 1. Insulin is a 51 amino acid hormone containing two peptide chains (i.e., a B-chain and an A-chain) connected via disulfide bonds (e.g., disulfide bonds formed by one or more B-chain cysteine side chain thiols and one or more A-chain cysteine side chain thiols). The A-chain of insulin is 21 amino acids in length and the B-chain of insulin is 30 amino acids in length. In the native form of insulin, the A-chain contains one intrachain disulfide bond formed by two A-chain cysteine side chain thiols.

As used herein, the term “insulin” or “insulin polypeptide” encompasses mature insulin, preproinsulin, proinsulin, and naturally occurring insulin, or analogs thereof. In embodiments, an insulin polypeptide can be a full-length insulin polypeptide or a fragment thereof. In embodiments, an insulin polypeptide can comprise one or more fragments from mature insulin, preproinsulin, proinsulin, or naturally occurring insulin.

Insulin is normally constructed as a N-terminus—B-chain:C-chain:A-chain—C-terminus polypeptide, wherein the C-chain is cleaved in order to make it bioactive. For reference purposes, the sequence of the entire human insulin molecule including the C-chain (i.e., human proinsulin) is shown below with the C-chain shown in bold-faced type:

(SEQ ID NO: 4)
FVNQHLCGSHLVEALYLVCGERGFFYTPKTRREAEDLQVGQVELG
GGPGAGSLQPLALEGSLQKRGIVEQCCTSICSLYQLENYCN.

The transformation of the single-chain insulin polypeptide into a bioactive two-chain polypeptide is normally accomplished within the p-cells of the islets of Langerhans prior to glucose-stimulated insulin secretion by two endoproteases, Type I endoproteases, PC1 and PC3, that disrupt the C peptide-B chain connection and PC2, and a Type II endoprotease, that cleaves the C peptide-A chain bond at exactly the right sites. However, cell systems used for the biosynthesis of therapeutic molecules such as insulin (e.g., bacteria, yeast, and mammalian (e.g., HEK and CHO) cell systems) do not possess this pathway, and therefore the transformation must take place after expression and harvesting of the single chain polypeptide using chemical or enzymatic methods. Known techniques for cleaving the C-chain after expression and harvesting rely on first modifying the C-chain such that it terminates in a lysine just before the N-terminus of the A-chain. Then, using an enzyme selected from the trypsin or Lys-C families, which clips peptide bonds specifically at the C-termini of lysine residues, the single chain-insulin polypeptide is cleaved at the C-terminal lysine of the C-chain and at the C-terminal lysine at the 29th position from the N-terminus of the B-chain. In some cases, the resulting bioactive two-chain insulin is used without reattaching the clipped amino acid at the 30th position from the N-terminus of the B-chain, and in some cases the clipped amino acid at the 30th position from the N-terminus of the B-chain is added back to the molecule using an additional enzymatic method. Such a process works well with insulin because it contains only one lysine in its entire two chain polypeptide form.

Recombinant human insulin (which in the present application is herein referred to as “RHI”) is a bioactive two-chain polypeptide comprising the B-chain of SEQ ID NO: 5: FVNQHLCGSHLVEALYLVCGERGFFYTPKT, and the A-chain of SEQ ID NO: 9: GIVEQCCTSICSLYQLENYCN connected by two disulfide bonds derived from cysteine residues (A7-B7 and A20-B19). A third disulfide is an intrachain disulfide bond derived from cysteine residues on the A-chain (A6-A11). This structure of RHI is well known in the art (see for example Brange, Jens, Gelanics of Insulin: The Physico-Chemical and Pharmaceutical Aspects of Insulin and Insulin Preparations (1987) Springer-Verlag Berlin Heidelberg, https://doi.org/10.1007/978-3-662-02526-0).

However, this process cannot be used on the insulin-Fc fusion proteins contained herein, because all known Fc fragments contain multiple lysine residues. The enzymatic cleavage process would, therefore, digest the Fc fragment into non-functional parts, thereby eliminating the ability of the Fc fragment to prolong the action of the insulin polypeptide in vivo. Therefore, an insulin-Fc fusion protein of the present invention must comprise an insulin polypeptide that does not require C-chain cleavage and is therefore bioactive in its single chain form.

A number of bioactive single chain insulin polypeptides have been described in the art. In all cases, the single chain insulin polypeptides contain C-chains of specific length and composition as well as A-chains and B-chains mutated at specific amino acid sites in order to achieve electrostatic balance, prevent aggregation, and enhance IR binding and/or downstream signaling to achieve bioactivity at levels comparable to that of the native two-chain insulin. Herein, the location of mutations on peptide segments are notated using the name of the segment (e.g., B-chain, C-chain, A-chain) and the number of the amino acid counting from the N-terminus of the segment. For example, the notation “B10” refers to the 10th amino acid from the N-terminus of the amino acid sequence of the B-chain. The notation “A8” refers to the 8th amino acid from the N-terminus of the A-chain. Furthermore, if an amino acid is mutated from its native form to a new amino acid at a particular location, the location is appended with the one letter amino acid code for the new amino acid. For example, B10D refers to an aspartic acid mutation at the 10th amino acid from the N-terminus of the amino acid sequence of the B-chain and A8H refers to a histidine mutation at the 8th amino acid from the N-terminus of the amino acid sequence of the A-chain.

In some embodiments, the insulin polypeptides of the present disclosure comprise insulin analogs. The insulin analogs may be closely related to the structure of insulin yet contain a modification (e.g., a structural modification) to enhance a certain functional aspect. In some embodiments, the insulin analog comprises a variant or mutant of insulin. In some embodiments, the insulin analog comprises at least one amino acid substitution, deletion, or addition relative to insulin.

In some embodiments, modifications to the sequence or structure of insulin or an insulin analog (e.g., an amino acid substitution, deletion, or addition, or a chemical modification) may impact a particular feature or characteristic of the insulin-Fc fusion protein (e.g., insulin-Fc fusion protein described herein). For example, the modifications or alterations described herein may impact the structure, stability, pH sensitivity, bioactivity, or binding affinity of the insulin-Fc fusion protein to a cell surface receptor (e.g., an insulin hormone receptor). In some embodiments, an amino acid substitution, addition, deletion, or a chemical modification relative to insulin may affect the activity of the insulin analog relative to a reference standard.

In embodiments, the insulin or insulin analog is a three-segment peptide comprising elements of a B-chain, a C-peptide, and an A-chain. In other embodiments, an insulin-Fc fusion protein described herein comprises an insulin polypeptide comprising a mutant insulin B-chain, C-peptide, and/or A-chain.

In embodiments, modifications to the sequence of the insulin or insulin analog (e.g., amino acid substitutions, deletions, or additions or chemical modifications) may be to either the B-chain of insulin, the C-peptide of insulin, the A-chain of insulin, or any combination thereof.

### Fc Fragment

Insulin-Fc fusion proteins combine an insulin polypeptide with a human Fc region as illustrated in FIG. 1. These insulin-Fc fusion proteins are made biologically in mammalian cells as a single chain, in which the insulin molecule is connected between the A- and B-chains with a short peptide sequence, and the use of a human Fc region acts to prolong their action in vivo.

The terms “Fc region,” “Fc domain,” “Fc polypeptide,” or “Fc fragment” as used herein are used to define a C-terminal region of an immunoglobulin heavy chain. The Fc fragment, region, or domain may be a native sequence Fc region or a variant/mutant Fc region. Although the boundaries of the Fc region of an immunoglobulin heavy chain may vary, they generally comprise some or all of the hinge region of the heavy chain, the CH2 region of the heavy chain, and the CH3 region of the heavy chain. The hinge region of a human Fc fragment comprises amino acid sequences that connect the CH1 domain of the heavy chain to the CH2 region of the heavy chain and which contain one or more cysteines that form one or more interheavy chain disulfide bridges to form a homodimer of the Fc fusion protein from two identical but separate monomers of the Fc fusion protein. The hinge region may comprise all or part of a naturally occurring amino acid sequence or a non-naturally occurring amino acid sequence.

An Fc receptor (FcR) refers to a receptor that binds to an Fc fragment or the Fc region of an antibody. In embodiments, the FcR is a native sequence human FcR. In embodiments, the FcR is one which binds an Fc fragment or the Fc region of an IgG antibody (a gamma receptor) and includes without limitation, receptors of the FcγRI, FcγRIIa, FcγRIIb, and FcγRIII subclasses, including allelic variants and alternatively spliced forms of these receptors. “FcR” also includes the neonatal receptor, FcRn, which is responsible for the transfer of maternal IgGs to the fetus (Guyer et al., 1976 J. Immunol., 117:587; and Kim et al., 1994, J. Immunol., 24:249) and is also responsible for the prolonged in vivo elimination half-lives of antibodies and Fc-fusion proteins in vivo. In embodiments, an Fc fragment described herein is capable of binding to mammalian Fc(gamma) or Fc(Rn) receptors, e.g., human Fc(gamma) or human Fc(Rn) receptors.

In embodiments, the C-terminal lysine that is often found in native human IgG isotype Fc fragment amino acid sequences (i.e., the lysine that represents the last amino acid of the Fc fragment sequence) is omitted to prevent the accidental production of unwanted amino acid sequence variants during manufacturing (e.g., Fc fragments containing the C-terminal lysine becoming mixed with Fc fragments where the C-terminal lysine is omitted, which can occur during production of the desired protein within cells (Dick, L W., (2008) Biotechnol Bioeng. August 15; 100(6) pp 1132-43).

In embodiments, the Fc fragment comprises the Fc region, e.g., hinge region, CH2 domain, and CH3 domain (or a fragment thereof) of a human immunoglobulin (e.g., IgG1). In embodiments, the Fc fragment comprises the hinge region (or a fragment thereof) of a human IgG1. In embodiments, the Fc fragment comprises the Fc region, e.g., CH2 domain and CH3 domain (or a fragment thereof) of human IgG1.

### Linker

In embodiments, a fusion protein described herein comprises a linker, e.g., between one or more domains of the polypeptide. For example, a fusion protein comprises a linker between the insulin polypeptide and the Fc fragment.

In some examples, the C-terminus of the insulin polypeptide is connected directly to the N-terminus of the Fc fragment (e.g., no linker or linker absent). In other examples, the successful construction of a recombinantly made insulin-Fc fusion protein requires a linker connecting the insulin polypeptide to the Fc fragment. In embodiments, the linker is a peptide. In embodiments, insulin-Fc fusion protein configurations described herein comprise a peptide linker between the insulin polypeptide and the Fc fragment comprising amino acids (e.g., natural, or unnatural amino acids). In embodiments, the peptide linker can be encoded by a nucleic acid molecule, for example such that a single nucleic acid molecule can encode the various peptides within an insulin polypeptide as well as the peptide linker and the Fc fragment. The choice of peptide linker (for example, the length, composition, hydrophobicity, and secondary structure) could impact the manufacturability of the insulin-Fc fusion protein configuration (i.e., the homodimer titer), the chemical and enzymatic stability, the bioactivity, parameters that correlate with bioactivity (i.e., the FcRn assay EC50 value), and the immunogenicity of the insulin-Fc fusion protein (Chen, X., Zaro, J., Shen, W. C., Adv Drug Deliv Rev. 2013 October 15; 65(10): 1357-1369).

### Fusion Proteins

Provided herein are fusion proteins, e.g., insulin-Fc fusion proteins. In embodiments, the fusion protein comprises an insulin polypeptide described herein, e.g., in the Insulin polypeptide section herein. In embodiments, the fusion protein comprises an Fc fragment, e.g., an Fc fragment described herein, e.g., in the Fc fragment section herein.

In embodiments, the fusion protein comprises a linker described, e.g., in the Linker section herein between the insulin polypeptide described, e.g., in the Insulin polypeptide section herein and the Fc fragment described, e.g., in the Fc fragment section herein.

In embodiments, the insulin polypeptide comprises domains in the following orientation from N- to C-termini: (N-terminus)—B-chain—C-peptide—A-chain—(C-terminus). The insulin polypeptide may be located at the N-terminus of the Fc domain.

In embodiments, the fusion protein comprises domains in the following orientation from N- to C-termini: (N-terminus)—insulin polypeptide—linker—Fc fragment—(C-terminus) (e.g., (N-terminus)—B-chain—C-peptide—A-chain—linker—Fc fragment—(C-terminus); or (N-terminus)—B-chain-C-peptide—A-chain—linker—Fc fragment—(C-terminus)) as illustrated in FIG. 1. In embodiments, the fusion protein, also referred to as the insulin-Fc fusion protein, is comprised of two identical insulin-Fc fusion proteins covalently bound together via one or more disulfide bonds (shown as dotted lines in FIG. 1; the total number of disulfide bonds in actuality may be greater or less than the number shown in FIG. 1). Each insulin-Fc fusion protein comprises a proinsulin-like insulin molecule containing an insulin B-chain and an insulin A-chain that are connected between the B-chain-C-terminal region and the A-chain-NH2 terminal region with a C-chain (gray line in FIG. 1), and the A-chain-C-terminal region and Fc-chain amino terminus with a linker, where the insulin-Fc fusion protein sequence terminates in the Fc-CH3 region-C-terminal region. Note that the B-chain and A-chain are also linked together via two disulfide bonds (dotted lines in FIG. 1). The A-chain also has an intramolecular disulfide bond (not shown in FIG. 1).

### The Treatment of Cancer Tumors Through Prolonged Hypoglycemia

Cancer cells consume increased amounts of glucose compared to normal cells, metabolizing the glucose-derived pyruvate to lactate even in the presence of oxygen (the Warburg effect). While aerobic glycolysis is less efficient (in terms of adenosine triphosphate production) than mitochondrial oxidative phosphorylation that normal cells use to produce energy, it does lead to the increased generation of additional metabolites that benefit proliferating cells such as cancer cells. As the Warburg effect is associated with glucose uptake and utilization, it was envisioned that an ultra-long-acting basal insulin for diabetes treatment that would effectively lower blood sugar for a prolonged period of time would be useful in treating cancer. Insulin-analog mutants that can bind and activate the insulin hormone receptor and take advantage of FcRn receptor recycling to prolong their action could accomplish this goal through prolonged interaction with insulin receptors present on cell surfaces to help arrest cancer cell growth and mitogenesis.

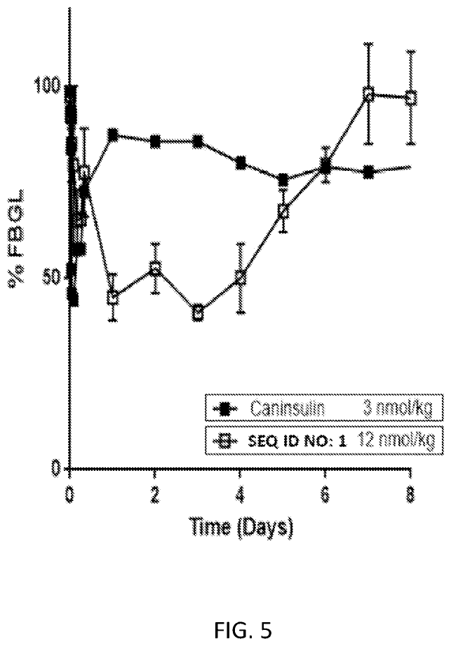

Insulin-Fc fusion proteins comprising mutations in the A- and B-chains and covalently linked through a peptide linker to an IgG Fc region were required to accomplish this goal. Variations of insulin-Fc fusion proteins were designed with the expectation that compounds with similar properties could bind and activate the insulin hormone receptor and do so with an acceptably low insulin receptor affinity EC50 ratio as compared to the affinity of endogenous insulin, in addition to taking advantage of the FcRn receptor recycling to substantially increase the half-life of the compounds in serum. Achieving these outcomes, for example in the insulin-Fc fusion protein of SEQ ID NO: 1, required including a peptide sequence between the A- and B-chains, in addition to requiring specific mutations in the A- and B-chains themselves. The insulin-Fc fusion protein of SEQ ID NO: 1, in addition to demonstrating high affinity for the insulin receptor (as described in Example 6a) demonstrated acceptable potency and protracted blood glucose lowering activity in mice as described in Example 8a and shown in FIG. 5.

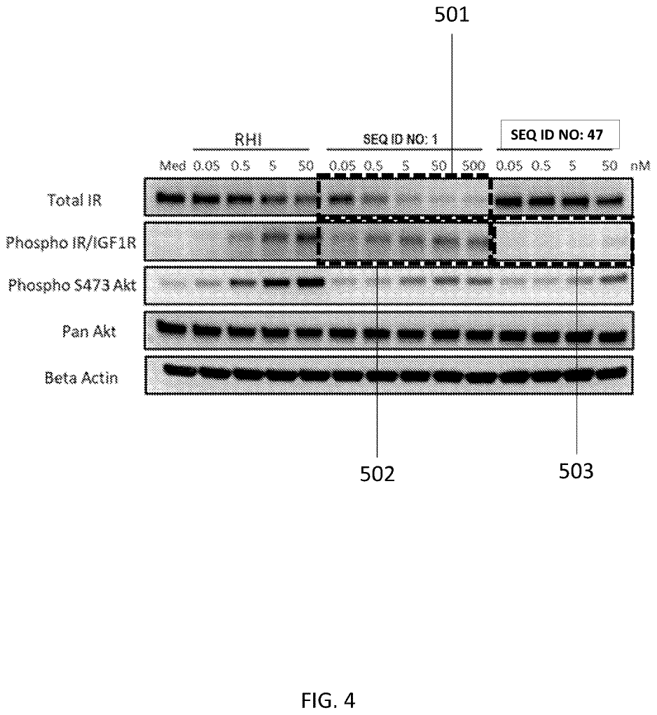

Unexpectedly, it was observed in mice treated with the insulin-Fc fusion protein of SEQ ID NO: 1 that even though the mice had blood glucose (BG) levels significantly lower than normal (as shown in FIG. 5 with a comparison to a porcine insulin NPH vehicle), they did not exhibit clinical signs of hypoglycemia such as lethargy, loss of balance, convulsions, or loss of consciousness even when fasted for prolonged periods of time. Given these unexpected results, it was hypothesized that the insulin-Fc fusion protein of SEQ ID NO: 1 may be able to slow cancerous tumor growth by inducing “managed hypoglycemia” thereby limiting the glucose available to cancer cells for aerobic glycolysis, while sparing the rest of the body. The insulin-Fc fusion protein of SEQ ID NO: 1 demonstrated the ability to downregulate the insulin receptor in HCT-116 cells vivo (as described in Example 7a and shown in FIG. 4).

Testing in nude mice according to Example 9a performed in HCT-116 xenograft models (HCT-116 cells are used in a variety of biomedical studies involving colon cancer proliferation and corresponding inhibitors) and according to Example 10a with an in vivo model of a metastatic human melanoma cell line (WM266.4), demonstrated that the insulin-Fc fusion protein of SEQ ID NO: 1 was capable of slowing tumor growth compared to controls under fasted conditions. Animals treated with conventional NPH insulin showed no benefit with treatment under fasted conditions, even though similar fasting levels of hypoglycemia were observed in both NPH and the insulin-Fc fusion protein of SEQ ID NO: 1 after dosing, and higher doses of NPH were not feasible due to frequent incidences of life-threatening hypoglycemia in the mice. Unexpectedly, animals treated with the insulin-Fc fusion protein of SEQ ID NO: 1 without fasting still exhibited significant reduction in tumor growth rate. The data indicated that prolonged hypoglycemia was not the mechanism by which the insulin-Fc fusion protein of SEQ ID NO: 1 was inhibiting cancer cell growth.

### Treatment of Cancer Tumors Through Decreasing Insulin-Like Growth Factor 1 Receptor (IGF1R)

The IGF1 receptor or IFG1R is a transmembrane receptor found on the surface of cells. Because the IGF1R is overexpressed in several tumor types and as a result of its impact on tumor survival and proliferation in preclinical studies, several anti-IGF1R therapies have been developed for clinical trials. While promising, these therapies have had limited success in the clinic, most likely due to the development of resistance through alternate signaling pathways.

Still further, it is known that treatment of some cancers with particular drugs (e.g., treatment of breast cancers with a drug such as Tamoxifen) can result in breast cancer cells with reduced or downregulated IFG1R. This downregulation of IFG1R eventually allows the cancer cells to become resistant to the drug (e.g., drug induced resistance, or “Tamoxifen-resistant” cancers or tumors) through alternate signaling pathways similar to what has occurred in anti-IGF1R therapy approaches. This drug-induced resistance has been demonstrated in the laboratory with cancerous cell lines (e.g., breast cancer cell line MCF-7 and Tamoxifen resistant breast cancer cell line MCF-7 (also known as MCF-7 TamR).

The IGF1R is activated by a hormone called insulin-like growth factor 1 (IGF-1) and by a related hormone called insulin-like growth factor 2 (IGF-2). Ligand binding of IGF-1 and IGF-2 to the IGF1R on the surface of cells leads to autophosphorylation and activation of two distinct but overlapping pathways: PI3K-Akt and the MAPK. The PI3K pathway is a cascade which leads to phosphorylation and activation of Akt, a serine/threonine kinase, which regulates cellular metabolism through the translocation of the GLUT4 glucose transporter to the cell surface. The fully activated Akt mediates downstream responses including cell survival, growth, proliferation, cell migration and angiogenesis, by phosphorylating a range of intracellular proteins, regulating cell survival through inhibition of apoptosis, and making it important for tumor survival. Activation of the MAPK pathway causes the activation of ERK1/2, leading to increased cell proliferation, metastasis, and tumor growth.

Clinicians would like to treat certain cancers by decreasing the IGF1R present on tumors, based on the hypothesis that less IGF-1 and IGF-2 would be capable of binding the IFG1R and triggering the downstream cell proliferation and growth of the tumors. Approaches using anti-IGF1R antibody therapies have been tried in the clinic, many of which have advanced as far as Phase 3 clinical trials, but all programs have been discontinued due to the tumors building resistance over time. When IGF1R is downregulated in response to binding the therapeutically administered anti-IGF1R antibodies and internalization of the receptor-antibody complex, IGF-1 and IGF-2 serum concentrations increase as they are not being eliminated through the IGF1R as quickly. IGF-1 and IGF-2 are capable of activating the insulin receptor (IR) at high concentrations due to their structural similarity to insulin, resulting in binding and signaling through the IR. This undesirable activation of the IR/phosphorylated-Akt signals the tumor to continue proliferating. Thus IGF-2 signaling through the IR is a potential mechanism of resistance for IFG1R therapies.

One way to prevent IGF-2 signaling through the IR is to use anti-IR antibodies in combination with anti-IGF1R antibodies. However there have only been a limited number of clinical candidates targeting the IR due to anti-IR antibodies resulting in downregulation of IR, which leads to decreased insulin binding leading to unwanted hyperglycemia and insulin resistance.

Another approach focuses on short interfering RNA (siRNA) or RNA interference (RNAi). A promoter system may be used to deliver and express siRNA targeting IGF1R to reduce its expression in cells. This downregulation of IGF1R results in significant inhibition of cancer cell growth in vitro and in vivo in rodents. However, this approach is also likely to suffer from upregulated IGF-1 and IGF-2 binding and activation of IR leading to tumor growth and unwanted hyperglycemia. A further approach is to use small molecule, tyrosine kinase inhibitors (TKIs) that simultaneously target both the IGF1R and IR systems without having to blockade the receptors themselves or reduce the receptor expression levels. However, because TKIs are small molecules, they lack specificity for IGF1R/IR and can therefore potentially disrupt other receptor systems, including those not involved in cancer cell metabolism, causing unwanted side effects and toxicity as a result. Furthermore, TKIs also lead to unwanted hyperglycemia due to disruption of the IR pathway for glucose homeostasis. These various approaches support the assertion that preparations that downregulate both the IGF1R and the IR without unwanted side effects or hypo/hyperglycemia risks would significantly inhibit cancer cell growth resulting in desirable anti-tumor efficacy.

### Treatment of Cancer Tumors Through Downregulation of Insulin Receptor (IR)

To examine the relationship between IR binding and activation in vitro and tumor volume reduction in vivo, the IR activity of the insulin-Fc fusion protein of SEQ ID NO: 1 was tested in HCT-116 cells.

When tested according to the protocol of Example 7a the insulin-Fc fusion protein of SEQ ID NO: 1 and RHI caused substantial downregulation of IR in HCT-116 cells Unexpectedly, the insulin-Fc fusion protein of SEQ ID NO: 1 caused observably more downregulation than RHI at every concentration tested, suggesting that its bivalent homodimer structure, unique insulin mutations, and/or Fc component differentiate its behavior at the receptor level. Furthermore, the insulin-Fc fusion protein of SEQ ID NO: 1 induced lower levels of Akt phosphorylation than RHI at every concentration tested. Correlation of this data with the IR binding in vitro suggests that strong IR binding (relative to a reference insulin-Fc fusion protein) was necessary for receptor downregulation. The effects of both the insulin-Fc fusion protein of SEQ ID NO: 1 and RHI on the MAPK pathway were much more muted, with almost no change in phosphor-ERK1/2 expression after 72 hours of treatment.

The preliminary data indicate that compared to insulin, the insulin-Fc fusion protein of SEQ ID NO: 1 slightly decreases activation of the PI3K pathway, which plays an important role in cancer cell metabolism and survival. However, unlike TKIs and anti-IR antibodies, the insulin-Fc fusion protein of SEQ ID NO: 1 is not an antagonist. It allows enough signaling through this pathway to regulate blood glucose levels, which is shown in FIG. 4 for Phospho S473 Akt, which shows retained activation of the insulin receptor pathway even when the IR is being downregulated. Thus, the insulin-Fc fusion protein of SEQ ID NO: 1 is unique among IR targeting molecules in that it can inhibit tumor growth without causing hyperglycemia or insulin resistance. Thus, we hypothesize that the in vivo anti-tumor efficacy observed thus far with the insulin-Fc fusion protein of SEQ ID NO: 1 (and, conversely, not with insulin NPH) is likely related to one or more of the following effects on tumor cells: (i) downregulation of IR, (ii) downregulation of IGF1R, (iii) lower activation of the Akt pathway (e.g. less phosphorylated Akt in the presence of the insulin-Fc fusion protein of SEQ ID NO: 1), (iv) downregulation of IR in combination with additional therapies that separately target downregulation of IGF1R, or (v) downregulation of IR in tumors that have low levels of IGF1R expression.

To attempt to determine whether specific mutations on the B-chain and A-chain of the insulin polypeptide contributed to the in vivo anti-tumor efficacy of the insulin-Fc fusion protein of SEQ ID NO: 1, the insulin-Fc fusion protein of SEQ ID NO: 47 was designed. As shown in Table A, the insulin-Fc fusion protein of SEQ ID NO: 47 comprises the B-chain of SEQ ID NO: 49 (FVNQHLCGSHLVEALELVCGERGFHY) which maintains the native histidine at B10 and the A-chain of SEQ ID NO: 51 (GIVEQCCTSTCSLDQLENYC) which maintains the native threonine at A8. The B-chain and A-chain are connected by the C-peptide of SEQ ID NO: 50 (GGGGGGSGGGG). The complete insulin polypeptide of the fusion protein SEQ ID NO: 47 is shown below as SEQ ID NO: 53.

(SEQ ID NO: 53)
FVNQHLCGSHLVEALELVCGERGFHYGGGGGGSGGGGGIVEQCCTSTCS
LDQLENYC

The insulin polypeptide of SEQ ID NO: 53 was linked via the linker of SEQ ID NO: 52 (GGGGGQGGGGQGGGGQGGGGG) to the human IgG1 fragment of SEQ ID NO: 13.

(SEQ ID NO: 13)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG.

The resulting insulin-Fc fusion protein of SEQ ID NO: 47 is given below.

(SEQ ID NO: 47)
FVNQHLCGSHLVEALELVCGERGFHYGGGGGGSGGGGGIVEQCCTSTCS
LDQLENYCGGGGGQGGGGQGGGGQGGGGGDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPG.

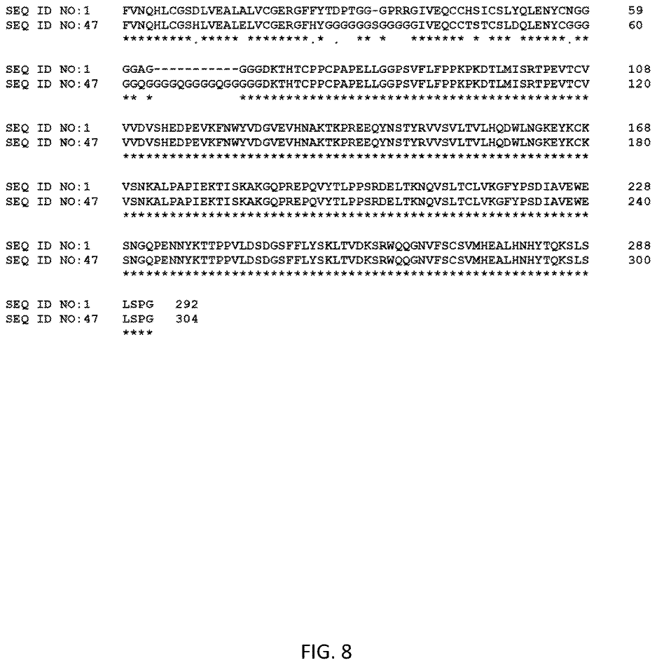

FIG. 8 illustrates a side-by-side sequence comparison of the insulin-Fc fusion protein of SEQ ID NO: 1 and the insulin-Fc fusion protein of SEQ ID NO: 47. “*” represents complete homology across all sequences at a given sequence position, while “:”, “.” or spaces refer to conservative, moderate, or very different amino acid mutations across the sequences at a given sequence position, respectively.

The insulin-Fc fusion protein of SEQ ID NO: 47 was manufactured according to Example 1a, resulting in an acceptable protein titer of 196.4 mg/L. The insulin-Fc fusion protein of SEQ ID NO: 47 was purified according to Example 2a and the structure of the insulin-Fc fusion protein of SEQ ID NO: 47 was confirmed according to Example 3a and is identified according to Example 4. The homodimer percentage was determined according to Example 5a to be acceptable at 97.8%.

The in vitro IM-9 insulin receptor (IR) binding of the insulin-Fc fusion protein of SEQ ID NO: 47 was tested according to Example 6a. Unexpectedly, as described in Example 6a, the insulin-Fc fusion protein of SEQ ID NO: 47 showed very low affinity to the insulin receptor in vitro.

When tested according to the protocol of Example 7a, unexpectedly the insulin-Fc fusion protein of SEQ ID NO: 47 did not cause more downregulation of IR than RHI at any concentration tested, as shown in FIG. 4, even though the insulin-Fc fusion protein of SEQ ID NO: 47 has the same bivalent homodimer structure as the insulin-Fe fusion protein of SEQ ID NO: 1. It was hypothesized that the one or more of the unique insulin mutations of B10D and A8H contained in the insulin-Fc fusion protein of SEQ ID NO: 1 and not in the insulin-Fc fusion protein of SEQ ID NO: 47 were necessary to differentiate the behavior of the insulin-Fc fusion protein at the insulin receptor level. Furthermore, the inability of the insulin-Fc fusion protein of SEQ ID NO: 47 to induce lower levels of Akt phosphorylation than RHI at any concentration tested (again as shown in FIG. 4) confirmed the correlation of this data with the IR binding in vitro, confirming that strong IR binding (relative to a reference insulin-Fc fusion protein) was necessary for receptor downregulation.

To confirm whether the specific mutations of B10D on the B-chain and A8H on the A-chain of the insulin polypeptide contributed to the in vivo anti-tumor efficacy of the insulin-Fc fusion protein of SEQ ID NO: 1, the insulin-Fc fusion proteins of the insulin-Fc fusion protein of SEQ ID NO: 16 was designed. As shown in Table A, the insulin Fc fusion protein of SEQ ID NO: 16 comprises the B-chain of SEQ ID NO: 35 which maintains the mutation at B10 from histidine to aspartic acid (B10D) from the insulin-Fc fusion protein of SEQ ID NO: 1, the C-Chain peptide sequence of SEQ ID NO: 8 as in the insulin-Fc fusion protein of SEQ ID NO: 1, and the A-Chain of SEQ ID NO: 10 which maintains the mutation at A8 from threonine to histidine (A8H) from the insulin-Fc fusion protein of SEQ ID NO: 1.

(SEQ ID ON: 35)
FVNQHLCGSDLVEALALGCGEEGFFYTDPT
(SEQ ID NO: 8)
GGGPRR
(SEQ ID NO: 10)
GIVEQCCHSICSLYQLENYCN

The resulting insulin polypeptide is given in SEQ ID NO: 41.

(SEQ ID NO: 41)
FVNQHLCGSDLVEALALVCGEEGFFYTDPTGGGPRRGIVEQCCHSICSL
YQLENYCN

The insulin polypeptide of SEQ ID NO: 41 is linked via the peptide linker of SEQ ID NO: 12 (GGGGSGGGG) to the human IgG1 Fc fragment of SEQ ID NO: 13.

(SEQ ID NO: 13)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG.

The resulting insulin-Fc fusion protein of SEQ ID NO: 16 is given below.

(SEQ ID NO: 16)
FVNQHLCGSDLVEALALVCGEEGFFYTDPTGGGPRRGIVEQCCHSICSL
YQLENYCNGGGGSGGGGDKTHTCPPCPAPELLGGPSVFLFPPKPKDTLM
ISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG

The insulin-Fc fusion protein of SEQ ID NO: 16 is manufactured according to Example 1b and purified according to Example 2b. The structure of the insulin-Fc fusion protein of SEQ ID NO: 16 is confirmed according to Example 3b and identified according to Example 4. The homodimer percentage is determined according to Example 5b.

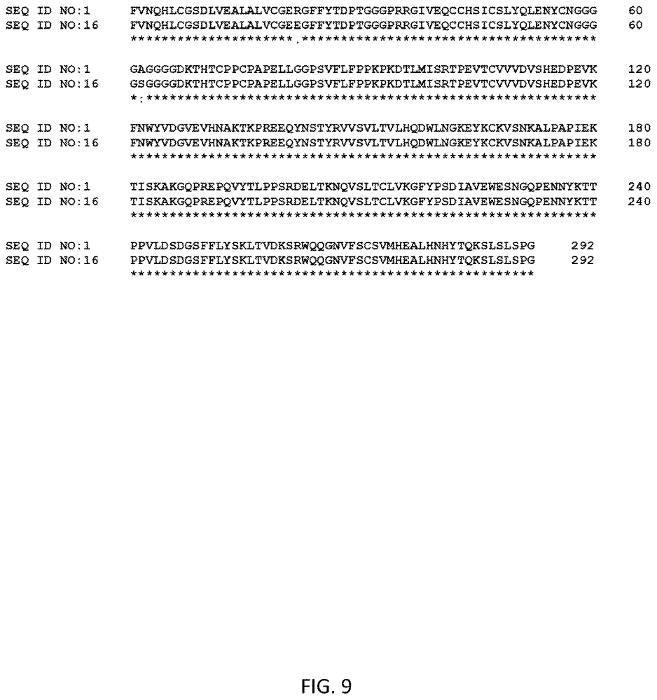

FIG. 9 illustrates a side-by-side sequence comparison of the insulin-Fc fusion protein of SEQ ID NO: 1 and the insulin-Fc fusion protein of SEQ ID NO: 16. “*” represents complete homology across all sequences at a given sequence position, while “:”, “.” or spaces refer to conservative, moderate, or very different amino acid mutations across the sequences at a given sequence position, respectively.

Testing of the insulin-Fc fusion protein of SEQ ID NO: 16 according to Example 6b is expected to reveal that like SEQ ID NO: 1, the insulin-Fc fusion protein of SEQ ID NO: 16 has strong affinity to the insulin receptor in vitro (where the strength of the affinity or binding to the insulin receptor is relative to a reference insulin-Fc fusion protein). When tested according to the protocol of Example 7b, the insulin-Fc fusion protein of SEQ ID NO: 16 is expected to cause substantial downregulation of IR in HCT-116 cells at every concentration tested and is expected to induce lower levels of Akt phosphorylation than RHI at every concentration tested.

Testing in nude mice according to Example 9b performed in HCT-116 xenograft models and testing according to Example 10b with an in vivo model of a metastatic human melanoma cell line (WM266.4) is expected to demonstrate that the insulin-Fc fusion protein of SEQ ID NO: 16 is capable of slowing tumor growth compared to controls under fasted and unfasted conditions.

In a further attempt to make insulin-Fc fusion proteins with strong insulin receptor affinity (where the strength of the affinity or binding to the insulin receptor is relative to a reference insulin-Fc fusion protein) and the ability to downregulate the insulin receptor in HCT-116 cells, two insulin-Fc fusion proteins with the A8H A-chain mutation were designed, where the B-chain was directly linked to the A-chain without a C-chain peptide linker. The first of these insulin-Fc-fusions was SEQ ID NO: 28. As shown in Table A, the insulin Fc fusion protein of SEQ ID NO: 28 comprises the B-chain of SEQ ID NO: 39 which conserves the native histidine at B10, has no C-Chain peptide sequence, and comprises the A-Chain of SEQ ID NO: 10 which maintains the mutation at A8 from threonine to histidine (A8H) from SEQ ID NO: 1.

FVNQHLCGSHLVEALALVCGEEGFFYTDK (SEQ ID NO: 39)
(SEQ ID NO: 10)
GIVEQCCHSICSLYQLENYCN

The resulting insulin polypeptide is given in SEQ ID NO: 45.

(SEQ ID NO: 45)
FVNQHLCGSHLVEALALVCGEEGFFYTDKGIVEQCCHSICSLYQLENY
CN

The insulin polypeptide of SEQ ID NO: 45 is linked via the peptide linker of SEQ ID NO: 11 (GGGGAGGGG) to the human IgG1 Fc fragment of SEQ ID NO: 34.

(SEQ ID NO: 34)
DQTHTCPPCPAPELLGGPSVFLFPPKPKDTLMISRTPEVTCVVVDVSHE
DPEVKFNWYVDGVEVHNAKTKPREEQYQSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG.

The resulting insulin-Fc fusion protein of SEQ ID NO: 28 is given below.

(SEQ ID NO: 28)
FVNQHLCGSHLVEALALVCGEEGFFYTDKGIVEQCCHSICSLYQLENYC
NGGGGAGGGGDQTHTCPPCPAPELLGGPSVFLFPPKPKDTLMISRTPEV
TCVVVDVSHEDPEVKFNWYVDGVEVHNAKTKPREEQYQSTYRVVSVLTV
LHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFL
YSKLTVDKSRWQQGNVFSCSVMHEALHNHYTQKSLSLSPG

The second insulin-Fc-fusion where the B-chain was directly linked to the A-chain without a C-chain peptide linker was SEQ ID NO: 30. As shown in Table A, the insulin-Fc fusion protein of SEQ ID NO: 30 comprises the B-chain of SEQ ID NO: 40 which conserves the native histidine at B10, has no C-Chain peptide sequence, and has the A-Chain of SEQ ID NO: 10 which maintains the mutation at A8 from threonine to histidine (A8H) from the insulin-Fc fusion protein of SEQ ID NO: 1.

FVNQHLCGSHLVEALALVCGEEGFFYTDR (SEQ ID NO: 40)
(SEQ ID NO: 10)
GIVEQCCHSICSLYQLENYCN

The resulting insulin polypeptide is given in SEQ ID NO: 45.

(SEQ ID NO: 45)
FVNQHLCGSHLVEALALVCGEEGFFYTDKGIVEQCCHSICSLYQLENY
CN

The insulin polypeptide of SEQ ID NO: 45 is linked via the peptide linker of SEQ ID NO: 11 (GGGGAGGGG) to the human IgG1 Fc fragment of SEQ ID NO: 34.

(SEQ ID NO: 34)
DQTHTCPPCPAPELLGGPSVFLFPPKPKDTLMISRTPEVTCVVVDVSHE
DPEVKFNWYVDGVEVHNAKTKPREEQYQSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG.

The resulting insulin-Fc fusion protein of SEQ ID NO: 30 is given below.

(SEQ ID NO: 30)
FVNQHLCGSHLVEALALVCGEEGFFYTDRGIVEQCCHSICSLYQLENYC
NGGGGAGGGGDQTHTCPPCPAPELLGGPSVFLFPPKPKDTLMISRTPEV
TCVVVDVSHEDPEVKFNWYVDGVEVHNAKTKPREEQYQSTYRVVSVLTV
LHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFL
YSKLTVDKSRWQQGNVFSCSVMHEALHNHYTQKSLSLSPG

The insulin-Fc fusion proteins of SEQ ID NO: 28 and SEQ ID NO: 30 were manufactured according to Example 1a. The resulting yields were acceptable and are shown in Table 1 below.

TABLE 1
Titer (mg/L) for Insulin-Fc Fusion Proteins
Manufactured in HEK293 Cells

| Sequence | Titer (mg/L) |
|---|---|
| SEQ ID NO: 28 | 72.4 |
| SEQ ID NO: 30 | 98.8 |

The insulin-Fc fusion proteins of SEQ ID NO: 28 and SEQ ID NO: 30 are purified according to Example 2b and the structure of the insulin-Fe fusion proteins of SEQ ID NO: 28 and SEQ ID NO: 30 is confirmed according to Example 3b and is identified according to Example 4. The homodimer percentage is determined according to Example 5b and is expected to be acceptable, at greater than 80%.

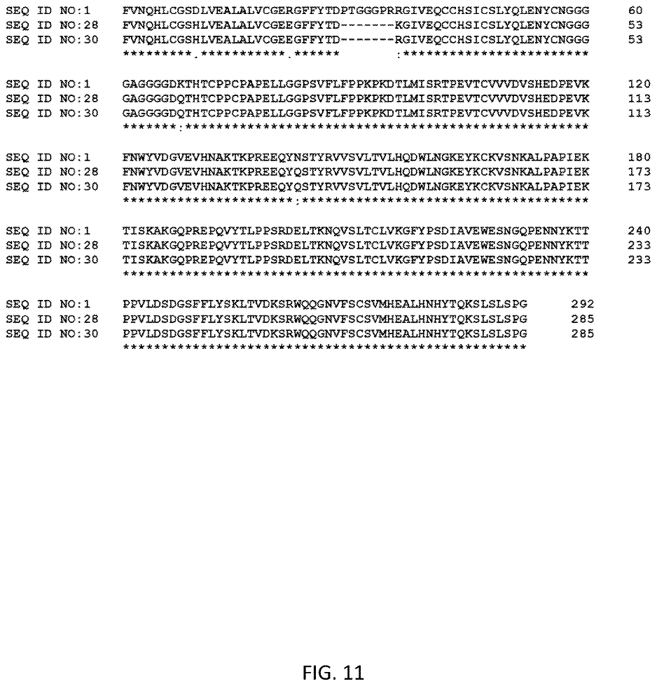

FIG. 11 illustrates a side-by-side sequence comparison of the insulin-Fc fusion protein of SEQ ID NO: 1, the insulin-Fc fusion protein of SEQ ID NO: 28 and the insulin-Fc fusion protein of SEQ ID NO 30. “*” represents complete homology across all sequences at a given sequence position, while “:”, “.” or spaces refer to conservative, moderate, or very different amino acid mutations across the sequences at a given sequence position, respectively.

Testing of the insulin-Fc fusion proteins of SEQ ID NO: 28 and SEQ ID NO: 30 according to Example 6b is expected to reveal that in spite of conserving the A8H mutation of the insulin-Fc fusion protein of SEQ ID NO: 1, the insulin-Fc fusion proteins of SEQ ID NO: 28 and SEQ ID NO: 30 do not have strong affinity to the insulin receptor in vitro (where the strength of the affinity or binding to the insulin receptor is relative to a reference insulin-Fc fusion protein). It is hypothesized that either the B10D mutation or the presence of the C-chain peptide between the B-chain and the A-chain, is a necessary part of the insulin-Fc fusion protein structure. When tested according to the protocol of Example 7b, the insulin-Fc fusion proteins of SEQ ID NO: 28 and SEQ ID NO: 30 are not expected to cause substantial downregulation of IR in HCT-116 cells at any concentration tested and are not expected to induce lower levels of Akt phosphorylation than RHI at any concentration tested.

Testing in nude mice according to Example 9b performed in HCT-116 xenograft models and testing according to Example 10b with an in vivo model of a metastatic human melanoma cell line (WM266.4) is expected to demonstrate that neither the insulin-Fc fusion protein of SEQ ID NO: 28 nor the insulin-Fc fusion protein of SEQ ID NO: 30 is capable of slowing tumor growth compared to controls under fasted or unfasted conditions.

Considering the expected results of the insulin-Fc fusion proteins of SEQ ID NO: 28 and SEQ ID NO: 30, in a further attempt to make an insulin-Fc fusion protein with strong insulin receptor affinity (where the strength of the affinity or binding to the insulin receptor is relative to a reference insulin-Fc fusion protein) and the ability to downregulate the insulin receptor in HCT-116 cells, three insulin-Fc fusion proteins were designed with the B10D B-chain mutation restored, in addition to the A8H A-chain mutation, and absent a C-chain peptide linker between the B-chain and the A-chain, that is where the B-chain was directly linked to the A-chain without a C-chain peptide linker. The first of these three Insulin-Fc fusion proteins is SEQ ID NO: 22. As shown in Table A, the insulin-Fc fusion protein of SEQ ID NO: 22 comprises the B-chain of SEQ ID NO: 36 which maintains the mutation at B10 from histidine to aspartic acid (B10D) from the insulin-Fc fusion protein of SEQ ID NO: 1, no C-Chain peptide sequence, and the A-Chain of SEQ ID NO: 10 which maintains the mutation at A8 from threonine to histidine (A8H) from the insulin-Fc fusion protein of SEQ ID NO: 1.

(SEQ ID NO: 36)
FVNQHLCGSDLVEALALVCGEEGFFYTDK
(SEQ ID NO: 10)
GIVEQCCHSICSLYQLENYCN

The resulting insulin polypeptide is given in SEQ ID NO: 42.

(SEQ ID NO: 42)
FVNQHLCGSDLVEALALVCGEEGFFYTDKGIVEQCCHSICS
LYQLENYCN

The insulin polypeptide of SEQ ID NO: 42 is linked via the peptide linker of SEQ ID NO: 11 (GGGGAGGGG) to the human IgG1 Fc fragment of SEQ ID NO: 34.

(SEQ ID NO: 34)
DQTHTCPPCPAPELLGGPSVFLFPPKPKDTLMISRTPEVTC
VVVDVSHEDPEVKFNWYVDGVEVHNAKTKPREEQYQSTYRV
VSVLTVLHQDWLNGKEYKCKVSNKALPAPIEKTISKAKGQP
REPQVYTLPPSRDELTKNQVSLTCLVKGFYPSDIAVEWESN
GQPENNYKTTPPVLDSDGSFFLYSKLTVDKSRWQQGNVFSC
SVMHEALHNHYTQKSLSLSPG.

The resulting insulin-Fc fusion protein of SEQ ID NO: 22 is given below.

(SEQ ID NO: 22)
FVNQHLCGSDLVEALALVCGEEGFFYTDKGIVEQCCHSICS
LYQLENYCNGGGGAGGGGDQTHTCPPCPAPELLGGPSVFLF
PPKPKDTLMISRTPEVTCVVVDVSHEDPEVKFNWYVDGVEV
HNAKTKPREEQYQSTYRVVSVLTVLHQDWLNGKEYKCKVSN
KALPAPIEKTISKAKGQPREPQVYTLPPSRDELTKNQVSLT
CLVKGFYPSDIAVEWESNGQPENNYKTTPPVLDSDGSFFLY
SKLTVDKSRWQQGNVFSCSVMHEALHNHYTQKSLSLSPG

The second of the three Insulin-Fe fusion proteins with the B10D B-chain mutation restored, in addition to the A8H A-chain mutation, and absent a C-chain peptide linker between the B-chain and the A-chain is SEQ ID NO: 24. As shown in Table A, the insulin-Fc fusion protein of SEQ ID NO: 24 comprises the B-chain of SEQ ID NO: 37 which maintains the mutation at B10 from histidine to aspartic acid (B10D) from the insulin-Fc fusion protein of SEQ ID NO: 1, no C-Chain peptide sequence, and the A-Chain of SEQ ID NO: 10 which maintains the mutation at A8 from threonine to histidine (A8H) from the insulin-Fc fusion protein of SEQ ID NO: 1.

(SEQ ID NO: 37)
FVNQHLCGSDLVEALALVCGEAGFFYTDK
(SEQ ID NO: 10)
GIVEQCCHSICSLYQLENYCN

The resulting insulin polypeptide is given in SEQ ID NO: 43.

(SEQ ID NO: 43)
FVNQHLCGSDLVEALALVCGEAGFFYTDKGIVEQCCHSICS
LYQLENYCN

The insulin polypeptide of SEQ ID NO: 43 is linked via the peptide linker of SEQ ID NO: 11 (GGGGAGGGG) to the human IgG1 Fe fragment of SEQ ID NO: 34.

(SEQ ID NO: 34)
DQTHTCPPCPAPELLGGPSVFLFPPKPKDTLMISRTPEVTC
VVVDVSHEDPEVKFNWYVDGVEVHNAKTKPREEQYQSTYRV
VSVLTVLHQDWLNGKEYKCKVSNKALPAPIEKTISKAKGQP
REPQVYTLPPSRDELTKNQVSLTCLVKGFYPSDIAVEWESN
GQPENNYKTTPPVLDSDGSFFLYSKLTVDKSRWQQGNVFSC
SVMHEALHNHYTQKSLSLSPG.

The resulting insulin-Fc fusion protein of SEQ ID NO: 24 is given below.

(SEQ ID NO: 24)
FVNQHLCGSDLVEALALVCGEAGFFYTDKGIVEQCCHSICS
LYQLENYCNGGGGAGGGGDQTHTCPPCPAPELLGGPSVFLF
PPKPKDTLMISRTPEVTCVVVDVSHEDPEVKFNWYVDGVEV
HNAKTKPREEQYQSTYRVVSVLTVLHQDWLNGKEYKCKVSN
KALPAPIEKTISKAKGQPREPQVYTLPPSRDELTKNQVSLT
CLVKGFYPSDIAVEWESNGQPENNYKTTPPVLDSDGSFFLY
SKLTVDKSRWQQGNVFSCSVMHEALHNHYTQKSLSLSPG

The third of the three Insulin-Fc fusion proteins with the B10D B-chain mutation restored, in addition to the A8H A-chain mutation, and absent a C-chain peptide linker between the B-chain and the A-chain is SEQ ID NO: 26. As shown in Table A, the insulin-Fc fusion protein of SEQ ID NO: 26 comprises the B-chain of SEQ ID NO: 38 which maintains the mutation at B10 from histidine to aspartic acid (B10D) from SEQ ID NO: 1, no C-Chain peptide sequence, and the A-Chain of SEQ ID NO: 10 which maintains the mutation at A8 from threonine to histidine (A8H) from the insulin-Fc fusion protein of SEQ ID NO: 1.

(SEQ ID NO: 38)
FVNQHLCGSDLVEALALVCGEEGFFYTDR
(SEQ ID NO: 10)
GIVEQCCHSICSLYQLENYCN

The resulting insulin polypeptide is given in SEQ ID NO: 44.

(SEQ ID NO: 44)
FVNQHLCGSDLVEALALVCGEEGFFYTDRGIVEQCCHSICS
LYQLENYCN

The insulin polypeptide of SEQ ID NO: 44 is linked via the peptide linker of SEQ ID NO: 11 (GGGGAGGGG) to the human IgG1 Fc fragment of SEQ ID NO: 34.

(SEQ ID NO: 34)
DQTHTCPPCPAPELLGGPSVFLFPPKPKDTLMISRTPEVTC
VVVDVSHEDPEVKFNWYVDGVEVHNAKTKPREEQYQSTYRV
VSVLTVLHQDWLNGKEYKCKVSNKALPAPIEKTISKAKGQP
REPQVYTLPPSRDELTKNQVSLTCLVKGFYPSDIAVEWESN
GQPENNYKTTPPVLDSDGSFFLYSKLTVDKSRWQQGNVFSC
SVMHEALHNHYTQKSLSLSPG.

The resulting insulin-Fc fusion protein of SEQ ID NO: 26 is given below.

(SEQ ID NO: 26)
FVNQHLCGSDLVEALALVCGEEGFFYTDRGIVEQCCHSICS
LYQLENYCNGGGGAGGGGDQTHTCPPCPAPELLGGPSVFLF
PPKPKDTLMISRTPEVTCVVVDVSHEDPEVKFNWYVDGVEV
HNAKTKPREEQYQSTYRVVSVLTVLHQDWLNGKEYKCKVSN
KALPAPIEKTISKAKGQPREPQVYTLPPSRDELTKNQVSLT
CLVKGFYPSDIAVEWESNGQPENNYKTTPPVLDSDGSFFLY
SKLTVDKSRWQQGNVFSCSVMHEALHNHYTQKSLSLSPG

The insulin-Fc fusion proteins of SEQ ID NO: 22, SEQ ID NO: 24 and SEQ ID NO: 26 were manufactured according to Example 1a. The resulting yields were acceptable and are shown in Table 2 below.

TABLE 2
Titer (mg/L) for Insulin-Fc Fusion Proteins
Manufactured in HEK293 Cells

| Sequence | Titer (mg/L) |
|---|---|
| SEQ ID NO: 22 | 107 |
| SEQ ID NO: 24 | 119 |
| SEQ ID NO: 26 | 106 |

The insulin-Fc fusion proteins of SEQ ID NO: 22, SEQ ID NO: 24 and SEQ ID NO: 26 are purified according to Example 2b and the structure of the insulin-Fc fusion proteins of SEQ ID NO: 22, SEQ ID NO: 24 and SEQ ID NO: 26 are confirmed according to Example 3b and identified according to Example 4. The homodimer percentage is determined according to Example 5b and is expected to be acceptable, at greater than 80%.

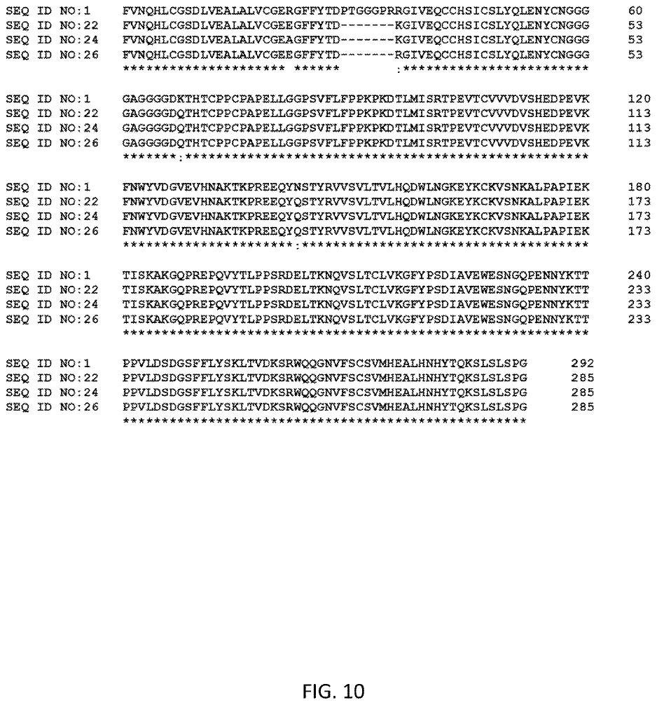

FIG. 10 illustrates a side-by-side sequence comparison of the insulin-Fc fusion proteins of SEQ ID NO: 1, SEQ ID NO: 22, SEQ ID NO: 24 and SEQ ID NO 26. “*” represents complete homology across all sequences at a given sequence position, while “:”, “.” or spaces refer to conservative, moderate, or very different amino acid mutations across the sequences at a given sequence position, respectively.

Unexpectedly, testing of the insulin-Fc fusion proteins of SEQ ID NO: 22, SEQ ID NO: 24 and SEQ ID NO: 26 according to Example 6b is expected to reveal that in spite of conserving both the B10D and A8H mutations of the insulin-Fc fusion protein of SEQ ID NO: 1, the insulin-Fc fusion proteins of the insulin-Fc fusion proteins of SEQ ID NO: 22, SEQ ID NO: 24 and SEQ ID NO: 26 do not have strong affinity to the insulin receptor in vitro (where the strength of the affinity or binding to the insulin receptor is relative to a reference insulin-Fc fusion protein). According, it is believed that the presence of the C-chain peptide between the B-chain and the A-chain is a necessary part of the insulin-Fc fusion protein structure to obtain strong affinity to the insulin receptor (where the strength of the affinity or binding to the insulin receptor is relative to a reference insulin-Fc fusion protein). When tested according to the protocol of Example 7b, the insulin-Fc fusion proteins of SEQ ID NO: 22, SEQ ID NO: 24 and SEQ ID NO: 26 are not expected to cause substantial downregulation of IR in HCT-116 cells at any concentration tested and are not expected to induce lower levels of Akt phosphorylation than RHI at any concentration tested.

Testing in nude mice according to Example 9b performed in HCT-116 xenograft models and testing according to Example 10b with an in vivo model of a metastatic human melanoma cell line (WM266.4) is expected to demonstrate that none of the insulin-Fc fusion protein of SEQ ID NO: 22, the insulin-Fc fusion protein of SEQ ID NO: 24 nor the insulin-Fc fusion protein of SEQ ID NO: 26 is capable of slowing tumor growth compared to controls under fasted or unfasted conditions.

As previously shown, the insulin-Fc fusion protein of SEQ ID NO: 1 exhibited strong affinity to the insulin receptor (where the strength of the affinity or binding to the insulin receptor is relative to a reference insulin-Fc fusion protein) and an ability to downregulate the insulin receptor in HCT-116 cells, and the insulin-Fc fusion protein of SEQ ID NO: 16 is expected to achieve the same results. However, on previous experiments of insulin-Fc fusion proteins with dogs and cats, it was unexpectedly discovered that the specific amino acids mutations at B10D and A8H that resulted in strong insulin receptor binding (where the strength of the affinity or binding to the insulin receptor is relative to a reference insulin-Fc fusion protein), additionally led to the development of neutralizing anti-drug antibodies after repeated subcutaneous injections in the target animal (e.g., dog or cat). The anti-drug antibodies led to an unacceptable reduction in the ability of the insulin-Fc fusion protein to interact with the insulin receptor after multiple injections, rendering the associated insulin-Fc fusion proteins non-viable for ongoing treatments. Specifically, it was discovered in the steps leading up to the invention of this disclosure that the A8 mutation to histidine and the B10 mutation to aspartic acid accounted for the vast majority of the anti-drug antibody specificity and thus represented immunogenic “hot spots” (e.g., immunogenic epitopes) on the insulin-polypeptide. The insulin-Fc fusion proteins of SEQ ID NO: 1 and SEQ ID NO: 16 are tested to assess their propensity to cause anti-drug antibodies according to Example 12. It is expected that significant inhibition of anti-drug antibody signals will be observed in the drug-inhibit wells, meaning that the anti-drug antibodies are specific to the therapeutic compounds.

The insulin-Fc fusion proteins of SEQ ID NO: 1 and SEQ ID NO: 16 are further analyzed for immunogenic epitope identification according to Example 13. It is expected that by correlating the resulting antibody concentrations from the assay with the known amino acid compositions of the coated insulin-Fc fusion protein library, it will be determined that the insulin acid mutations at position 10 on the insulin B-chain (B10D) and position 8 on the insulin A-chain (A8H) are responsible for some, most, or all of the total antibody signal on the assay, indicating weak or strong binding to various insulin-Fc fusion protein homodimers. It is expected that the amino acid mutations at position 10 on the insulin B-chain (B10D) and position 8 on the insulin A-chain (A8H) will be seen to be immunogenic “hot spots”.

In a further attempt to maintaining the efficacy of the insulin polypeptide of SEQ ID NO: 1, which was shown to have strong affinity to the insulin receptor (where the strength of the affinity or binding to the insulin receptor is relative to a reference insulin-Fc fusion protein), without the risk of anti-drug antibody generation, deglycosylating or preventing the glycosylation of the Fc fragment during synthesis in the host cell is desirable. The human IgG1 fragment contains a conserved asparagine (N)-glycosylation site in the CH2 domain of each heavy chain of the Fc region. Herein, the notation used to refer to the conserved N-glycosylation site is “cNg”. One way to remove the attached glycan from a synthesized insulin-Fc fusion protein is to mutate the cNg site to prevent the attachment of glycans altogether during production in the host cell. Herein, the notation used to describe a cNg mutation is cNg-(substituted amino acid). For example, if the asparagine at the cNg site is mutated to serine, this mutation is notated as “cNg-S”.

Maintaining the insulin polypeptide of the insulin-Fc fusion protein of SEQ ID NO: 1, which was shown to have strong affinity to the insulin receptor (where the strength of the affinity or binding to the insulin receptor is relative to a reference insulin-Fc fusion protein), an insulin-Fc fusion protein of SEQ ID NO: 18 was designed. The insulin polypeptide of the insulin-Fc fusion protein of SEQ ID NO: 18 includes mutations in the B-chain from the native human RHI insulin (SEQ ID NO: 5). Specifically, B10 is mutated to aspartic acid (D), B16 is mutated to Alanine (A), B28 is mutated to aspartic acid (D) and B29 is mutated to proline (P). The B-chain (SEQ ID NO: 7) of the insulin-Fc fusion protein of SEQ ID NO: 18 is given below. Additionally, there is a mutation in the A-chain of the insulin-Fc fusion protein of SEQ ID NO: 18 (specifically, A8 is mutated to histidine (H)), also shown below:

(SEQ ID NO: 7)
FVNQHLCGSDLVEALALVCGERGFFYTDPT
(SEQ ID NO: 10)
GIVEQCCHSICSLYQLENYCN

The B-chain of the insulin-Fc fusion protein and the A-chain of the insulin-Fc fusion protein of SEQ ID NO: 18 are joined via a C-chain peptide which comprises the amino acid sequence GGGPRR (SEQ ID NO: 8). The resulting insulin polypeptide is shown below:

(SEQ ID NO: 6)
FVNQHLCGSDLVEALALVCGERGFFYTDPTGGGPRRGIVEQ
CCHSICSLYQLENYCN

The Fc fragment of the insulin-Fc fusion protein of SEQ ID NO: 18 has a mutation at the conserved glycosylation site, cNg, from asparagine to serine (cNg-S). The Fc fragment of the insulin-Fc fusion protein of SEQ ID NO: 18 is SEQ ID NO: 32 and is shown below.

(SEQ ID NO: 32)
DKTHTCPPCPAPELLGGPSVFLFPPKPKDTLMISRTPEVTC
VVVDVSHEDPEVKFNWYVDGVEVHNAKTKPREEQYSSTYRV
VSVLTVLHQDWLNGKEYKCKVSNKALPAPIEKTISKAKGQP
REPQVYTLPPSRDELTKNQVSLTCLVKGFYPSDIAVEWESN
GQPENNYKTTPPVLDSDGSFFLYSKLTVDKSRWQQGNVFSC
SVMHEALHNHYTQKSLSLSPG

The Fc fragment (SEQ ID NO: 32) of the insulin-Fc fusion protein of SEQ ID NO: 18 is connected to the insulin polypeptide (SEQ ID NO: 6) of the insulin-Fc fusion protein of SEQ ID NO: 18 via the peptide linker GGGGAGGGG (SEQ ID NO: 11) to create the insulin-Fc fusion protein of SEQ ID NO: 18. The B10D and A8H mutations on the B-chain and A-chain respectively of the insulin polypeptide of the insulin-Fc fusion protein of SEQ ID NO: 18 were demonstrated through experimentation with other insulin-Fc fusion proteins to be necessary mutations to achieve an insulin receptor binding affinity that is high enough to achieve insulin receptor downregulation on cell surfaces. Furthermore in vivo studies are expected to show that the insulin-Fc fusion protein of SEQ ID NO: 18 achieves the proper glycemic control without hyperglycemia or hypoglycemia in vivo. In examples, the required necessary insulin receptor binding affinity is achieved when the fusion protein achieves an IR binding IC₅₀ ratio relative to RHI of less than 20, as described in detail in Example 6a. The resulting insulin-Fc fusion protein of SEQ ID NO: 18 is given below.

(SEQ ID NO: 18)
FVNQHLCGSDLVEALALVCGERGFFYTDPTGGGPRRGIVEQCCHSICSLY
QLENYCNGGGGAGGGGDKTHTCPPCPAPELLGGPSVFLFPPKPKDTLMIS
RTPEVTCVVVDVSHEDPEVKFNWYVDGVEVHNAKTKPREEQYSSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPG

In a further attempt to maintaining the efficacy of the insulin polypeptide of the insulin-Fc fusion protein of SEQ ID NO: 1, which was shown to have strong affinity to the insulin receptor (where the strength of the affinity or binding to the insulin receptor is relative to a reference insulin-Fc fusion protein), without the risk of anti-drug antibody generation, an insulin-Fc fusion protein of SEQ ID NO: 20 was designed. The insulin polypeptide of the insulin-Fc fusion protein of SEQ ID NO: 20 includes mutations in the B-chain from the native human RHI insulin (SEQ ID NO: 5). Specifically, B10 is mutated to aspartic acid (D), B16 is mutated to Alanine (A), B28 is mutated to aspartic acid (D) and B29 is mutated to proline (P). The B-chain (SEQ ID NO: 7) of the insulin-Fe fusion protein of SEQ ID NO: 20 is given below. Additionally, there is a mutation in the A-chain of the insulin-Fc fusion protein of SEQ ID NO: 20 (specifically, A8 is mutated to histidine (H)), also shown below.

(SEQ ID NO: 7)
FVNQHLCGSDLVEALALVCGERGFFYTDPT
(SEQ ID NO: 10)
GIVEQCCHSICSLYQLENYCN

The B-chain of the insulin-Fc fusion protein and the A-chain of the insulin-Fc fusion protein of SEQ ID NO: 20 are joined via a C-chain peptide which comprises the amino acid sequence GGGPRR (SEQ ID NO: 8). The resulting insulin polypeptide is shown below:

(SEQ ID NO: 6)
FVNQHLCGSDLVEALALVCGERGFFYTDPTGGGPRRGIVEQCCHSICSLY
QLENYCN

The Fc fragment of the insulin-Fc fusion protein of SEQ ID NO: 20 has a mutation at the conserved glycosylation site, cNg, from asparagine to glutamine (cNg-Q). The Fc fragment of the insulin-Fc fusion protein of SEQ ID NO: 20 is SEQ ID NO: 33 and is shown below.

(SEQ ID NO: 33)
DKTHTCPPCPAPELLGGPSVFLFPPKPKDTLMISRTPEVTCVVVDVSHED
PEVKFNWYVDGVEVHNAKTKPREEQYQSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG

The Fc fragment (SEQ ID NO: 33) of the insulin-Fc fusion protein of SEQ ID NO: 20 is connected to the insulin polypeptide (SEQ ID NO: 6) of the insulin-Fc fusion protein of SEQ ID NO: 20 via the peptide linker GGGGAGGGG (SEQ ID NO: 11) to create the insulin-Fc fusion protein of SEQ ID NO: 20. The B10D and A8H mutations on the B-chain and A-chain respectively of the insulin polypeptide of the insulin-Fc fusion protein of SEQ ID NO: 20 were demonstrated through experimentation with other insulin-Fc fusion proteins to be necessary mutations to achieve an insulin receptor binding affinity that is high enough to achieve insulin receptor downregulation on cell surfaces. Furthermore in vivo studies are expected to show that the insulin-Fc fusion protein of SEQ ID NO: 20 achieves the proper glycemic control without hyperglycemia or hypoglycemia in vivo. In examples, the required necessary insulin receptor binding affinity is achieved when the fusion protein achieves an IR binding IC₅₀ ratio relative to RHI of less than 20, as described in detail in Example 6a. The resulting insulin-Fc fusion protein of SEQ ID NO: 20 is given below.

(SEQ ID NO: 20)
FVNQHLCGSDLVEALALVCGERGFFYTDPTGGGPRRGIVEQCCHSICSLY
QLENYCNGGGGAGGGGDKTHTCPPCPAPELLGGPSVFLFPPKPKDTLMIS
RTPEVTCVVVDVSHEDPEVKFNWYVDGVEVHNAKTKPREEQYQSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPG

The insulin-Fc fusion proteins of SEQ ID NO: 18 and SEQ ID NO: 20 are manufactured according to Example 1b and purified according to Example 2b. The structure of the insulin-Fc fusion proteins of SEQ ID NO: 18 and SEQ ID NO: 20 are confirmed according to Example 3b and identified according to Example 4. The homodimer percentage for each insulin-Fc fusion protein is determined according to Example 5b. Testing of the insulin-Fc fusion proteins of SEQ ID NO: 18 and SEQ ID NO: 20 according to Example 6b is expected to reveal that like the insulin-Fc fusion protein of SEQ ID NO: 1, the insulin-Fc fusion proteins of SEQ ID NO: 18 and SEQ ID NO: 20 have strong affinity to the insulin receptor in vitro (where the strength of the affinity or binding to the insulin receptor is relative to a reference insulin-Fc fusion protein).

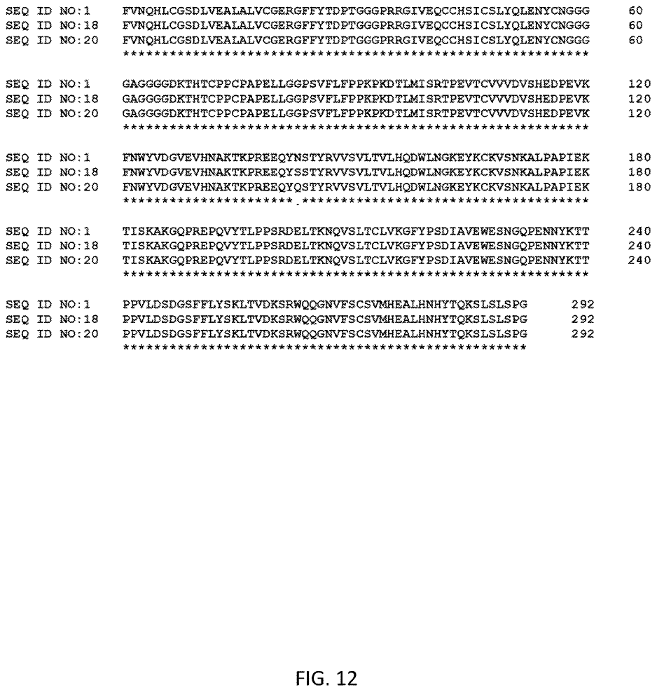

FIG. 12 illustrates a side-by-side sequence comparison of the insulin-Fc fusion proteins of SEQ ID NO: 1, SEQ ID NO: 18 and SEQ ID NO: 20. “*” represents complete homology across all sequences at a given sequence position, while “:”, “.” or spaces refer to conservative, moderate, or very different amino acid mutations across the sequences at a given sequence position, respectively.

Testing of the insulin-Fc fusion proteins of SEQ ID NO: 18 and SEQ ID NO: 20 according to Example 6b is expected to reveal that like the insulin-Fc fusion protein of SEQ ID NO: 1, the insulin-Fc fusion proteins of SEQ ID NO: 18 and SEQ ID NO: 20 have strong affinity to the insulin receptor in vitro (where the strength of the affinity or binding to the insulin receptor is relative to a reference insulin-Fc fusion protein). When tested according to the protocol of Example 7b, the insulin-Fc fusion proteins of SEQ ID NO: 18 and SEQ ID NO: 20 are expected to cause substantial downregulation of IR in HCT-116 cells at every concentration tested and are expected to induce lower levels of Akt phosphorylation than RHI at every concentration tested.

Testing in nude mice according to Example 9b performed in HCT-116 xenograft models and testing according to Example 10b with an in vivo model of a metastatic human melanoma cell line (WM266.4) is expected to demonstrate that the insulin-Fc fusion proteins of SEQ ID NO: 18 and SEQ ID NO: 20 are capable of slowing tumor growth compared to controls under fasted and unfasted conditions. Exemplary fusion proteins and their domains and sequences are shown in Table A.

The full-length sequences of fusion proteins designed in the description of the present technology and their corresponding cDNA sequences are provided below:

SEQ ID NO: 1:
FVNQHLCGSDLVEALALVCGERGFFYTDPTGGGPRRGIVEQCCHSICSLYQLENYCN
GGGGAGGGGDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQK
SLSLSPG
SEQ ID NO: 2 (cDNA sequence of SEQ ID NO: 1):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGCGGGGCTTCTTCTACACCGATCCCACTGGAGGCGGTCCACGCAGAG
GCATCGTGGAACAGTGCTGCCACTCCATCTGCTCCCTGTACCAGCTGGAAAACTA
CTGCAATGGCGGAGGTGGTGCAGGAGGCGGTGGAGACAAAACTCACACATGCCC
ACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGG
TGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAA
GCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGG
GTTAG
SEQ ID NO: 16:
FVNQHLCGSDLVEALALVCGEEGFFYTDPTGGGPRRGIVEQCCHSICSLYQLENYCN
GGGGSGGGGDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQK
SLSLSPG
SEQ ID NO: 17 (cDNA sequence of SEQ ID NO: 16):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGGAGGGCTTCTTCTACACCGATCCCACTGGAGGCGGTCCACGCAGAG
GCATCGTGGAACAGTGCTGCCACTCCATCTGCTCCCTGTACCAGCTGGAAAACTA
CTGCAATGGCGGAGGTGGTAGCGGAGGCGGTGGAGACAAAACTCACACATGCCC
ACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGG
TGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAA
GCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGG
GTTAG
SEQ ID NO: 18:
FVNQHLCGSDLVEALALVCGERGFFYTDPTGGGPRRGIVEQCCHSICSLYQLENYCN
GGGGAGGGGDKTHTCPPCPAPELLGGPSVFLFPPKPKDTLMISRTPEVTCVVVDVSHE
DPEVKFNWYVDGVEVHNAKTKPREEQYSSTYRVVSVLTVLHQDWLNGKEYKCKVS
NKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQK
SLSLSPG
SEQ ID NO: 19 (cDNA sequence of SEQ ID NO: 18):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGCGGGGCTTCTTCTACACCGATCCCACTGGAGGCGGTCCACGCAGAG
GCATCGTGGAACAGTGCTGCCACTCCATCTGCTCCCTGTACCAGCTGGAAAACTA
CTGCAATGGCGGAGGTGGTGCAGGAGGCGGTGGAGACAAAACTCACACATGCCC
ACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGG
TGGACGTGAGCCACGAAGACCCTGAGGTCAAGTTCAACTGGTACGTGGACGGCG
TGGAGGTGCATAATGCCAAGACAAAGCCGCGGGAGGAGCAGTACAGCAGCACGT
ACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAA
GCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGG
GTTAG
SEQ ID NO: 20:
FVNQHLCGSDLVEALALVCGERGFFYTDPTGGGPRRGIVEQCCHSICSLYQLENYCN
GGGGAGGGGDKTHTCPPCPAPELLGGPSVFLFPPKPKDTLMISRTPEVTCVVVDVSHE
DPEVKFNWYVDGVEVHNAKTKPREEQYQSTYRVVSVLTVLHQDWLNGKEYKCKVS
NKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQK
SLSLSPG
SEQ ID NO: 21 (cDNA sequence of SEQ ID NO: 20):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGCGGGGCTTCTTCTACACCGATCCCACTGGAGGCGGTCCACGCAGAG
GCATCGTGGAACAGTGCTGCCACTCCATCTGCTCCCTGTACCAGCTGGAAAACTA
CTGCAATGGCGGAGGTGGTGCAGGAGGCGGTGGAGACAAAACTCACACATGCCC
ACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGG
TGGACGTGAGCCACGAAGACCCTGAGGTCAAGTTCAACTGGTACGTGGACGGCG
TGGAGGTGCATAATGCCAAGACAAAGCCGCGGGAGGAGCAGTACCAGAGCACGT
ACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAA
GCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGG
GTTAG
SEQ ID NO: 22
FVNQHLCGSDLVEALALVCGEEGFFYTDKGIVEQCCHSICSLYQLENYCNGGGGAGG
GGDQTHTCPPCPAPELLGGPSVFLFPPKPKDTLMISRTPEVTCVVVDVSHEDPEVKFN
WYVDGVEVHNAKTKPREEQYQSTYRVVSVLTVLHQDWLNGKEYKCKVSNKALPAP
IEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG
SEQ ID NO: 23 (cDNA sequence of SEQ ID NO: 22):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGGAGGGCTTCTTCTACACCGACAAGGGCATCGTGGAACAGTGCTGCC
ACTCCATCTGCTCCCTGTACCAGCTGGAAAACTACTGCAATGGCGGAGGTGGTGC
AGGAGGCGGTGGAGACCAAACTCACACATGCCCACCGTGCCCAGCACCTGAACT
CCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGA
CAAAGCCGCGGGAGGAGCAGTACCAAAGCACGTACCGTGTGGTCAGCGTCCTCA
CCGTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGT
GCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACA
ACCACTACACGCAGAAGAGCCTCTCCCTGTCTCCGGGTTAG
SEQ ID NO: 24:
FVNQHLCGSDLVEALALVCGEAGFFYTDKGIVEQCCHSICSLYQLENYCNGGGGAG
GGGDQTHTCPPCPAPELLGGPSVFLFPPKPKDTLMISRTPEVTCVVVDVSHEDPEVKF
NWYVDGVEVHNAKTKPREEQYQSTYRVVSVLTVLHQDWLNGKEYKCKVSNKALP
APIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSP
G
SEQ ID NO: 25 (cDNA sequence of SEQ ID NO: 24):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGGCAGGCTTCTTCTACACCGACAAGGGCATCGTGGAACAGTGCTGCC
ACTCCATCTGCTCCCTGTACCAGCTGGAAAACTACTGCAATGGCGGAGGTGGTGC
AGGAGGCGGTGGAGACCAAACTCACACATGCCCACCGTGCCCAGCACCTGAACT
CCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGA
CAAAGCCGCGGGAGGAGCAGTACCAAAGCACGTACCGTGTGGTCAGCGTCCTCA
CCGTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGT
GCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACA
ACCACTACACGCAGAAGAGCCTCTCCCTGTCTCCGGGTTAG
SEQ ID NO: 26:
FVNQHLCGSDLVEALALVCGEEGFFYTDRGIVEQCCHSICSLYQLENYCNGGGGAGG
GGDQTHTCPPCPAPELLGGPSVFLFPPKPKDTLMISRTPEVTCVVVDVSHEDPEVKEN
WYVDGVEVHNAKTKPREEQYQSTYRVVSVLTVLHQDWLNGKEYKCKVSNKALPAP
IEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG
SEQ ID NO: 27 (cDNA sequence of SEQ ID NO: 26):
ATGGAATGGAGCTGGGTCTTTCTCTTCTTCCTGTCAGTAACGACTGGTGTCCACTC
CTTCGTGAACCAGCACCTGTGCGGCTCCGACCTGGTGGAAGCTCTGGCTCTCGTG
TGCGGCGAGGAGGGCTTCTTCTACACCGACCGAGGCATCGTGGAACAGTGCTGCC
ACTCCATCTGCTCCCTGTACCAGCTGGAAAACTACTGCAATGGCGGAGGTGGTGC
AGGAGGCGGTGGAGACCAAACTCACACATGCCCACCGTGCCCAGCACCTGAACT
CCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGA
CAAAGCCGCGGGAGGAGCAGTACCAAAGCACGTACCGTGTGGTCAGCGTCCTCA
CCGTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGT
GCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACA
ACCACTACACGCAGAAGAGCCTCTCCCTGTCTCCGGGTTAG
SEQ ID NO: 28:
FVNQHLCGSHLVEALALVCGEEGFFYTDKGIVEQCCHSICSLYQLENYCNGGGGAGG
GGDQTHTCPPCPAPELLGGPSVFLFPPKPKDTLMISRTPEVTCVVVDVSHEDPEVKFN
WYVDGVEVHNAKTKPREEQYQSTYRVVSVLTVLHQDWLNGKEYKCKVSNKALPAP
IEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG
SEQ ID NO: 29 (cDNA sequence of SEQ ID NO: 28):
ATGGAATGGAGCTGGGTCTTTCTCTTCTTCCTGTCAGTAACGACTGGTGTCCACTC
CTTCGTGAACCAGCACCTGTGCGGCTCCCACCTGGTGGAAGCTCTGGCTCTCGTG
TGCGGCGAGGAGGGCTTCTTCTACACCGACAAGGGCATCGTGGAACAGTGCTGCC
ACTCCATCTGCTCCCTGTACCAGCTGGAAAACTACTGCAATGGCGGAGGTGGTGC
AGGAGGCGGTGGAGACCAAACTCACACATGCCCACCGTGCCCAGCACCTGAACT
CCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGA
CAAAGCCGCGGGAGGAGCAGTACCAAAGCACGTACCGTGTGGTCAGCGTCCTCA
CCGTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGT
GCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACA
ACCACTACACGCAGAAGAGCCTCTCCCTGTCTCCGGGTTAG
SEQ ID NO: 30:
FVNQHLCGSHLVEALALVCGEEGFFYTDRGIVEQCCHSICSLYQLENYCNGGGGAGG
GGDQTHTCPPCPAPELLGGPSVFLFPPKPKDTLMISRTPEVTCVVVDVSHEDPEVKFN
WYVDGVEVHNAKTKPREEQYQSTYRVVSVLTVLHQDWLNGKEYKCKVSNKALPAP
IEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG
SEQ ID NO: 31 (cDNA sequence of SEQ ID NO: 30):
ATGGAATGGAGCTGGGTCTTTCTCTTCTTCCTGTCAGTAACGACTGGTGTCCACTC
CTTCGTGAACCAGCACCTGTGCGGCTCCCACCTGGTGGAAGCTCTGGCTCTCGTG
TGCGGCGAGGAGGGCTTCTTCTACACCGACAGGGGCATCGTGGAACAGTGCTGCC
ACTCCATCTGCTCCCTGTACCAGCTGGAAAACTACTGCAATGGCGGAGGTGGTGC
AGGAGGCGGTGGAGACCAAACTCACACATGCCCACCGTGCCCAGCACCTGAACT
CCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGA
CAAAGCCGCGGGAGGAGCAGTACCAAAGCACGTACCGTGTGGTCAGCGTCCTCA
CCGTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGT
GCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACA
ACCACTACACGCAGAAGAGCCTCTCCCTGTCTCCGGGTTAG
SEQ ID NO: 47:
FVNQHLCGSHLVEALELVCGERGFHYGGGGGGSGGGGGIVEQCCTSTCSLDQLENY
CGGGGGQGGGGQGGGGQGGGGGDKTHTCPPCPAPELLGGPSVFLFPPKPKDTLMIS
RTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVF
SCSVMHEALHNHYTQKSLSLSPG
SEQ ID NO: 48 (cDNA sequence of SEQ ID NO: 47):
ATGGAATGGAGCTGGGTCTTTCTCTTCTTCCTGTCAGTAACGACTGGTGTCCACTC
CTTCGTGAACCAGCACCTGTGCGGCTCCCACCTGGTGGAAGCTCTGGAACTCGTG
TGCGGCGAGCGGGGCTTCCACTACGGGGGTGGCGGAGGAGGTTCTGGTGGCGGC
GGAGGCATCGTGGAACAGTGCTGCACCTCCACCTGCTCCCTGGACCAGCTGGAAA
ACTACTGCGGTGGCGGAGGTGGTCAAGGAGGCGGTGGACAGGGTGGAGGTGGGC
AGGGAGGAGGCGGGGGAGACAAAACTCACACATGCCCACCGTGCCCAGCACCTG
AACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGC
AGCCCCGAGAACCACAGGTGTACACCCTGCCCCCATCCCGGGATGAGCTGACCA
AGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCC
CGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGC
ACAACCACTACACGCAGAAGAGCCTCTCCCTGTCTCCGGGTTAG

TABLE A
Insulin-Fc fusion protein sequences and their constituent parts

| Fusion | | | | | |
|---|---|---|---|---|---|
| protein | | | | | |
| SEQ ID | B-chain | C-peptide | A-chain | Linker aa | |
| NO: | aa seq | aa seq | aa seq | seq | Fc fragment aa seq |
| 1 | FVNQHLCGS | GGGPRR | GIVEQCC | GGGGAGG | DKTHTCPPCPAPELLGGPSVFLFPPKPK |
| | DLVEALALV | (SEQ ID | HSICSLY | GG | DTLMISRTPEVTCVVVDVSHEDPEVKF |
| | CGERGFFYT | NO: 8) | QLENYCN | (SEQ ID | NWYVDGVEVHNAKTKPREEQYNSTY |
| | DPT | | (SEQ ID | NO: 11) | RVVSVLTVLHQDWLNGKEYKCKVSN |
| | (SEQ ID NO: | | NO: 10) | | KALPAPIEKTISKAKGQPREPQVYTLPP |
| | 7) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 13) |
| 16 | FVNQHLCGS | GGGPRR | GIVEQCC | GGGGSGG | DKTHTCPPCPAPELLGGPSVFLFPPKPK |
| | DLVEALALV | (SEQ ID | HSICSLY | GG | DTLMISRTPEVTCVVVDVSHEDPEVKF |
| | CGEEGFFYT | NO: 8) | QLENYCN | (SEQ ID | NWYVDGVEVHNAKTKPREEQYNSTY |
| | DPT | | (SEQ ID | NO: 12) | RVVSVLTVLHQDWLNGKEYKCKVSN |
| | (SEQ ID NO: | | NO: 10) | | KALPAPIEKTISKAKGQPREPQVYTLPP |
| | 35) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 13) |
| 18 | FVNQHLCGS | GGGPRR | GIVEQCC | GGGGAGG | \|DKTHTCPPCPAPELLGGPSVFLFPPKPK |
| | DLVEALALV | (SEQ ID | HSICSLY | GG | DTLMISRTPEVTCVVVDVSHEDPEVKF |
| | CGERGFFYT | NO: 8) | QLENYCN | (SEQ ID | NWYVDGVEVHNAKTKPREEQYSSTY |
| | DPT | | (SEQ ID | NO: 11) | RVVSVLTVLHQDWLNGKEYKCKVSN |
| | (SEQ ID NO: | | NO: 10) | | KALPAPIEKTISKAKGQPREPQVYTLPP |
| | 7) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 32) |
| 20 | FVNQHLCGS | GGGPRR | GIVEQCC | GGGGAGG | DKTHTCPPCPAPELLGGPSVFLFPPKPK |
| | DLVEALALV | (SEQ ID | HSICSLY | GG | DTLMISRTPEVTCVVVDVSHEDPEVKF |
| | CGERGFFYT | NO: 8) | QLENYCN | (SEQ ID | NWYVDGVEVHNAKTKPREEQYQSTY |
| | DPT | | (SEQ ID | NO: 11) | RVVSVLTVLHQDWLNGKEYKCKVSN |
| | (SEQ ID NO: | | NO: 10) | | KALPAPIEKTISKAKGQPREPQVYTLPP |
| | 7) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 33) |
| 22 | FVNQHLCGS | No C- | GIVEQCC | GGGGAGG | DQTHTCPPCPAPELLGGPSVFLFPPKPK |
| | DLVEALALV CGEEGFFYT | Peptide | HSICSLY | GG | DTLMISRTPEVTCVVVDVSHEDPEVKF |
| | DK | | QLENYCN | (SEQ ID | NWYVDGVEVHNAKTKPREEQYQSTY |
| | (SEQ ID NO: | | (SEQ ID | NO: 11) | RVVSVLTVLHQDWLNGKEYKCKVSN |
| | 36) | | NO: 10) | | KALPAPIEKTISKAKGQPREPQVYTLPP |
| | | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 34) |
| 24 | FVNQHLCGS | No C- | GIVEQCC | GGGGAGG | DQTHTCPPCPAPELLGGPSVFLFPPKPK |
| | DLVEALALV | Peptide | HSICSLY | GG | DTLMISRTPEVTCVVVDVSHEDPEVKF |
| | CGEAGFFYT | | QLENYCN | (SEQ ID | NWYVDGVEVHNAKTKPREEQYQSTY |
| | DK | | (SEQ ID | NO: 11) | RVVSVLTVLHQDWLNGKEYKCKVSN |
| | (SEQ ID NO: | | NO: 10) | | KALPAPIEKTISKAKGQPREPQVYTLPP |
| | 37) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 34 ) |
| 26 | FVNQHLCGS | No C- | GIVEQCC | GGGGAGG | \|DQTHTCPPCPAPELLGGPSVFLFPPKPK |
| | DLVEALALV | Peptide | HSICSLY | GG (SEQ ID | DTLMISRTPEVTCVVVDVSHEDPEVKF |
| | CGEEGFFYT | | QLENYCN | NO: 11) | NWYVDGVEVHNAKTKPREEQYQSTY |
| | DR | | (SEQ ID | | RVVSVLTVLHQDWLNGKEYKCKVSN |
| | (SEQ ID NO: | | NO: 10) | | KALPAPIEKTISKAKGQPREPQVYTLPP |
| | 38) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 34) |
| 28 | FVNQHLCGS | No C- | GIVEQCC | GGGGAGG | DQTHTCPPCPAPELLGGPSVFLFPPKPK |
| | HLVEALALV | Peptide | HSICSLY | GG | DTLMISRTPEVTCVVVDVSHEDPEVKF |
| | CGEEGFFYT | | QLENYCN | (SEQ ID | NWYVDGVEVHNAKTKPREEQYQSTY |
| | DK | | (SEQ ID | NO: 11) | RVVSVLTVLHQDWLNGKEYKCKVSN |
| | (SEQ ID NO: | | NO: 10) | | KALPAPIEKTISKAKGQPREPQVYTLPP |
| | 39) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 34) |
| 30 | FVNQHLCGS | No C- | GIVEQCC | GGGGAGG | DQTHTCPPCPAPELLGGPSVFLFPPKPK |
| | HLVEALALV | Peptide | HSICSLY | GG | DTLMISRTPEVTCVVVDVSHEDPEVKF |
| | CGEEGFFYT | | QLENYCN | (SEQ ID | NWYVDGVEVHNAKTKPREEQYQSTY |
| | DR | | (SEQ ID | NO: 11) | RVVSVLTVLHQDWLNGKEYKCKVSN |
| | (SEQ ID NO: | | NO: 10) | | KALPAPIEKTISKAKGQPREPQVYTLPP |
| | 40) | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 34) |
| 47 | FVNQHLCGS | GGGGGG | GIVEQCC | GGGGGQG | DKTHTCPPCPAPELLGGPSVFLFPPKPK |
| | HLVEALELV | SGGGG | TSTCSLD | GGGQGGG | DTLMISRTPEVTCVVVDVSHEDPEVKF |
| | CGERGFHY | (SEQ ID | QLENYC | GQGGGGG | NWYVDGVEVHNAKTKPREEQYNSTY |
| | (SEQ ID NO: | NO: 50) | (SEQ ID | (SEQ ID | RVVSVLTVLHQDWLNGKEYKCKVSN |
| | 49) | | NO: 51) | NO: 52) | KALPAPIEKTISKAKGQPREPQVYTLPP |
| | | | | | SRDELTKNQVSLTCLVKGFYPSDIAVE |
| | | | | | WESNGQPENNYKTTPPVLDSDGSFFL |
| | | | | | YSKLTVDKSRWQQGNVFSCSVMHEA |
| | | | | | LHNHYTQKSLSLSPG |
| | | | | | (SEQ ID NO: 13) |

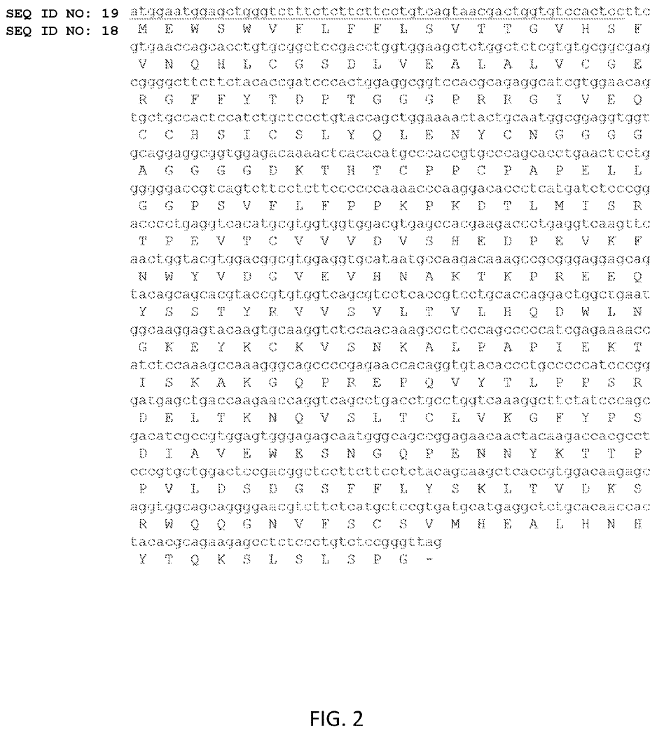

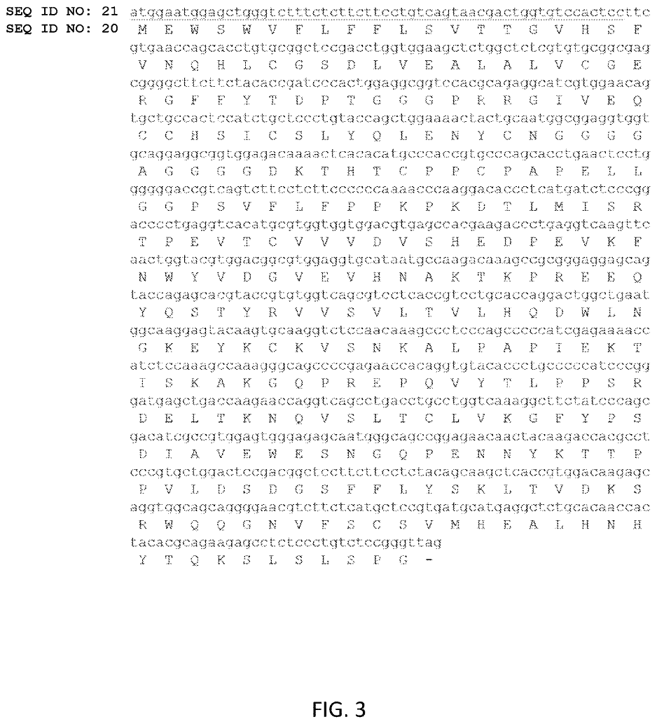

The “full aa sequences” of the insulin-Fc fusion proteins of SEQ ID NO: 18 (FIG. 2) and SEQ ID NO: 20 (FIG. 3) include a leader sequence. In embodiments, a fusion protein described herein does not include a leader sequence at the N-terminus. In embodiments, a fusion protein described herein includes a leader sequence, e.g., at the N-terminus. An exemplary leader sequence includes the amino acid sequence NMEWSWVFLFFLSVTTGVHIS (SEQ ID NO: 14). In embodiments, a fusion protein described herein is encoded by a nucleic acid molecule comprising a leader sequence, e.g., for expression (e.g., recombinant expression) in cells (e.g., eukaryotic, e.g., mammalian cells). In embodiments, the leader sequence is part of the fusion protein inside a cell and then the leader sequence is cleaved off, e.g., within the cell or in the cell culture, during expression of the fusion protein into the cell culture media via a process (e.g., an enzymatic process).

An exemplary nucleic acid sequence encoding a leader sequence includes the nucleic acid sequence:

(SEQ ID NO: 15)
ATGGAATGGAGCTGGGTCTTTCTCTTCTTCCTGTCAGTAACGACTGGTGT
CCACTCC.

In embodiments, a fusion protein described herein is encoded by a nucleic acid molecule not comprising a leader sequence.

In some embodiments, the fusion protein is in a preparation. In embodiments, the preparation has a percent dimer, e.g., homodimer, of the fusion protein that is greater than about 50%, e.g., greater than about 55%, about 60%, about 65%, about 70%, about 75%, about 80%, about 85%, about 90%, 95% or about 100%. In embodiments, the percent dimer, e.g., homodimer, of the fusion protein preparation is 91%, 92%, 93%, 94%, 95%, 96%, 97%, 98%, 99% or 100%. In embodiments, the percent homodimer is about 70% or higher (e.g., 80%, 85%, or 88% or more) and can be made 90% or higher (e.g., 95%, 97%, 98%, 99% or nearly 100%) using one or more processing steps (e.g., ion exchange chromatography, gel filtration, hydrophobic interaction chromatography, etc.). In some embodiments, the % dimer, e.g., homodimer, in the preparation is determined by size-exclusion chromatography (see Example 5a and Example 5b) which is an analytical separation method that can discriminate between dimers, e.g., homodimers, and higher-order non-covalent Fc fusion protein aggregates (e.g., multimers). In some embodiments, the % dimer, e.g., homodimer, is determined to be greater than 95%, e.g., as determined by size-exclusion chromatography. In some embodiments, the % dimer, e.g., homodimer, is determined to be greater than 99%, e.g., as determined by size-exclusion chromatography. In some embodiments, insulin-Fc fusion proteins with substantially greater homodimer content than other insulin-Fc fusion proteins demonstrate more bioactivity in a subject (e.g., a human).

### Fusion Protein Production

In embodiments, a fusion protein can be expressed by a vector as described in the Examples section.

### Expression and Purification

In embodiments, a fusion protein can be expressed recombinantly, e.g., in a eukaryotic cell, e.g., mammalian cell or non-mammalian cell. Exemplary mammalian cells used for expression include HEK cells, e.g., HEK293 cells, or CHO cells. In embodiments, cells are transfected with a nucleic acid molecule, e.g., vector, encoding the fusion protein (e.g., where the entire fusion protein is encoded by a single nucleic acid molecule). In other embodiments, cells are transfected with more than one nucleic acid molecule, where each nucleic acid molecule encodes a different domain of the fusion protein. For example, one nucleic acid molecule can encode the insulin polypeptide, and a different nucleic acid molecule can encode the Fc fragment. Cells can be cultured using standard methods in the art.

In some embodiments, the fusion protein is purified or isolated from the cells (e.g., by lysis of the cells). In other embodiments, the fusion protein is secreted by the cells and, e.g., the fusion protein is purified or isolated from the cell culture media in which the cells were grown. Purification of the fusion protein can include using column chromatography, e.g., affinity chromatography, or using other separation methods that involve size, charge, and/or affinity for certain molecules. In embodiments, purification of the fusion protein involves selecting or enriching for proteins with an Fc fragment, e.g., by using Protein A beads or a Protein A column that cause proteins containing an Fc fragment to become bound with high affinity at neutral solution pH to the Protein A covalently conjugated to the Protein A beads. The bound Fc fusion protein may then be eluted from the Protein A beads by a change in a solution variable (e.g., a decrease in the solution pH). Other separation methods such as ion exchange chromatography and/or gel filtration chromatography can also be employed alternatively or in addition. In embodiments, purification of the fusion protein further comprises filtering or centrifuging the protein preparation. In embodiments, further purification of the fusion protein comprises diafiltration, ultrafiltration, and filtration through porous membranes of various sizes, as well as final formulation with excipients.

The purified fusion protein can be characterized, e.g., for purity, yield, structure, and/or activity, using a variety of methods, e.g., absorbance at 280 nm (e.g., to determine yield), size exclusion or capillary electrophoresis (e.g., to determine the molecular weight, percent aggregation, and/or purity), mass spectrometry (MS) and/or liquid chromatography (LC-MS) (e.g., to determine purity), and/or ELISA (e.g., to determine extent of binding, e.g., affinity, to an anti-insulin antibody). Exemplary methods of characterization are also described in the Examples section.

### Functional Features of Fusion Proteins

Described herein are methods for interacting with the human insulin receptor to reduce cancer tumor growth rates in mammals. The methods comprise the administration of a fusion protein (e.g., fusion protein described herein) to a subject. In embodiments, a fusion protein described herein is capable of lowering glucose levels (e.g., blood glucose levels) after administration in a subject. In embodiments, the glucose lowering activity of the fusion protein is higher than that of an insulin reference standard. In some embodiments, the duration of activity of the fusion protein can be measured by a decrease, e.g., a statistically significant decrease, in blood glucose relative to a pre-dose level.

In embodiments, the duration of activity of the fusion protein (e.g., the time during which there is a statistically significant decrease in blood glucose level in a subject relative to a pre-dose level) is longer than about 2 hours. In embodiments, the duration of activity of the fusion protein (e.g., the time during which there is a statistically significant decrease in blood glucose level in a subject relative to a pre-dose level) is longer than about 2 hours, 6 hours, 9 hours, 12 hours, 18 hours, 1 day, 1.5 days, 2 days, 2.2 days, 2.5 days, 3 days, 5 days, 7 days, 8 days, 9 days, 10 days or longer. In embodiments, the duration of activity of the fusion protein (e.g., the time during which there is a statistically significant decrease in blood glucose level in a subject relative to a pre-dose level) is longer than that of an insulin reference standard or control formulation.

### Pharmaceutical Compositions and Routes of Administration

Provided herein are pharmaceutical compositions containing a fusion protein described herein that can be used to lower blood glucose in humans. The amount and concentration of the fusion protein in the pharmaceutical compositions, as well as the quantity of the pharmaceutical composition administered to a subject, can be selected based on clinically relevant factors, such as medically relevant characteristics of the subject (e.g. age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Formulations of the present disclosure include those suitable for parenteral administration. The phrases “parenteral administration” and “administered parenterally” as used herein means modes of administration other than enteral and topical administration, usually by intravenous or subcutaneous injection.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, e.g., Tween-like surfactants. In some embodiments, the pharmaceutical composition (e.g., as described herein) comprises a Tween-like surfactant, e.g., Tween-20 or Tween-80. In some embodiments, the pharmaceutical composition (e.g., as described herein) comprises a Tween-like surfactant, e.g., Tween-80, at a concentration between about 0.001% and about 2%, or between about 0.005% and about 0.1%, or between about 0.01% and about 0.5%.

In some embodiments, the fusion protein is administered as a bolus, infusion, or an intravenous push. In some embodiments, the fusion protein is administered through syringe injection, pump, pen, needle, or indwelling catheter. Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

### Dosages

Actual dosage levels of the fusion protein can be varied so as to obtain an amount of the active ingredient that is effective to achieve the desired therapeutic response for a particular subject. The selected dosage level will depend upon a variety of factors including the activity of the particular fusion protein employed, or the ester, salt or amide thereof, the route of administration, the time of administration, the rate of excretion of the particular compound being employed, the duration of the treatment, other drugs, compounds and/or materials used in combination with the particular fusion protein employed, the age, sex, weight, condition, general health and prior medical history of the human being treated, and like factors well known in the medical arts.

In general, a suitable dose of a fusion protein will be that amount of the fusion protein that is the lowest dose effective to produce a therapeutic effect. Such an effective dose will generally depend upon the factors described above. Generally, intravenous, and subcutaneous doses of the fusion protein for a subject will range from about 150 to about 1500 micrograms per kilogram of body weight per day.

The present disclosure contemplates formulation of the fusion protein in any of the aforementioned pharmaceutical compositions and preparations. Furthermore, the present disclosure contemplates administration via any of the foregoing routes of administration. One of skill in the art can select the appropriate formulation and route of administration based on the condition being treated and the overall health, age, and size of the patient being treated.

### EXAMPLES

The present technology is further illustrated by the following Examples, which should not be construed as limiting in any way.

### Example 1a: Synthesis and Methods of Making an Insulin-Fc Fusion Protein in HEK Cells

Insulin-Fc fusion proteins were synthesized as follows. A gene sequence of interest was constructed using proprietary software (LakePharma, Belmont, CA) and was cloned into a high expression mammalian vector. HEK293 cells were seeded in a shake flask 24 hours before transfection and were grown using serum-free chemically defined media. A DNA expression construct that encodes the insulin-Fc fusion protein of interest was transiently transfected into a 2 L suspension of HEK293 cells using the Syd Labs (Natick, MA) standard operating procedure for transient transfection. After 20 hours, cells were counted to determine the viability and viable cell count, and titer was measured by FortéBio® Octet® (Pall FortéBio LLC, Fremont, CA). Additional readings were taken throughout the transient transfection production run. The culture was harvested on or after day 5.

### Example 1b: Synthesis and Methods of Making an Insulin-Fc Fusion Protein in HEK Cells

Insulin-Fc fusion proteins are synthesized as follows. A gene sequence of interest is constructed using proprietary software (LakePharma, Belmont, CA) and is cloned into a high expression mammalian vector. HEK293 cells are seeded in a shake flask 24 hours before transfection and are grown using serum-free chemically defined media. A DNA expression construct that encodes the insulin-Fc fusion protein of interest is transiently transfected into a 2 L suspension of HEK293 cells using the Syd Labs (Natick, MA) standard operating procedure for transient transfection. After 20 hours, cells are counted to determine the viability and viable cell count, and titer is measured by FortéBio® Octet® (Pall FortéBio LLC, Fremont, CA). Additional readings are taken throughout the transient transfection production run. The culture is harvested on or after day 5.

### Example 1c: Synthesis and Methods of Making an Insulin-Fc Fusion Protein in CHO Cells

A CHO cell line is originally derived from CHO-K1 (LakePharma, Belmont, CA), and the endogenous glutamine synthetase (GS) genes are knocked out by recombinant technology using methods known in the art. Stable expression DNA vectors are designed and optimized for CHO expression and GS selection and incorporated into a high expression mammalian vector (LakePharma, Belmont, CA). The sequence of each completed construct is confirmed prior to initiating scale up experiments. The suspension-adapted CHO cells are cultured in a humidified 5% CO2 incubator at 37° C. in a chemically defined media (CD OptiCHO; Invitrogen, Carlsbad, CA). No serum or other animal-derived products are used in culturing the CHO cells.

Approximately 80 million suspension-adapted CHO cells, growing in CD OptiCHO media during the exponential growth phase, are transfected by electroporation using MaxCyte® STX® system (MaxCyte, Inc., Gaithersburg, MD) with 80 μg DNA to a create a stable CHO cell line for each insulin-Fc fusion protein (DNA construct contains the full-length sequence of the insulin-Fc fusion protein). After twenty-four hours, the transfected cells are counted and placed under selection for stable integration of the insulin-Fc fusion genes. The transfected cells are seeded into CD OptiCHO selection media containing between 0-100 μM methionine sulfoximine (MSX) at a cell density of 0.5×106 cells/mL in a shaker flask and are incubated at 37° C. with 5% C02. During a selection process, the cells are spun down and resuspended in fresh selection media every 2-3 days until the CHO stable pool recovered its growth rate and viability. The cell culture is monitored for growth and titer.

The cells are grown to 2.5×106 cells per mL. At the time of harvest for cell banking, the viability is to remain above 95%. The cells are then centrifuged, and the cell pellet resuspended in the CD OptiCHO media with 7.5% dimethyl sulfoxide (DMSO) to a cell count of 15×106 cells per mL per vial. Vials are cryopreserved for storage in liquid nitrogen.

A small-scale-up production is performed using the CHO cells as follows. The cells are scaled up for production in CD OptiCHO growth medium containing 100 μM MSX at 37° C. and fed every 2-4 days as needed, with CD OptiCHO growth medium supplemented with glucose and additional amino acids as necessary for approximately 14-21 days. The conditioned media supernatant harvested from the stable pool production run is clarified by centrifuge spinning. The protein is run over a Protein A (MabSelect, GE Healthcare, Little Chalfont, United Kingdom) column pre-equilibrated with binding buffer. Washing buffer is then passed through the column until the OD280 value (NanoDrop, Thermo Scientific) is measured to be at or near background levels. The insulin-Fc fusion protein is eluted using a low pH buffer, elution fractions are collected, and the OD280 value of each fraction is recorded. Fractions containing the target insulin-Fc fusion protein are pooled and optionally further filtered using a 0.2 μM membrane filter.

The cell line is optionally further subcloned to monoclonality and optionally further selected for high titer insulin-Fc-fusion protein-expressing clones using the method of limiting dilution, a method known to those skilled in the art. After obtaining a high titer, monoclonal insulin-Fc fusion protein-expressing cell line, production of the insulin-Fc fusion protein is accomplished as described above in growth medium without MSX, or optionally in growth medium containing MSX, to obtain a cell culture supernatant containing the recombinant, CHO-made, insulin-Fc fusion protein. The MSX concentration is optionally increased over time to exert additional selectivity for clones capable of yielding higher product titers.

### Example 2a: Purification of an Insulin-Fc Fusion Protein

Purification of an insulin-Fc fusion protein was performed as follows. Conditioned media supernatants containing the secreted Fc fusion protein were harvested from the transiently transfected HEK production runs and were clarified by centrifugation. The supernatant containing the desired insulin-Fc fusion protein was run over a Protein A column, washed with various wash buffers including 0.15-0.50 M sodium chloride, and then eluted using a low pH solution. Afterwards, the eluted desired protein fractions were pooled, and buffer exchanged into 200 mM HEPES, 100 mM NaCl, 50 mM NaOAc, pH 7.0 buffer. A final filtration step was performed using a 0.2 m membrane filter. The final protein concentration was calculated from the solution optical density at 280 nm. Further optional purification by ion-exchange chromatography (e.g., using an anion exchange bead resin or a cation exchange bead resin), gel filtration chromatography, or other methods was performed as necessary. In some embodiments, the insulin-Fc fusion was buffer exchanged via Zeba gel filtration columns (Thermo) into 50 mM sodium phosphate, pH 7.0 buffer and purified via Q-HP (Cytiva) ion exchange columns operating in flow through mode to remove molecular aggregates, host cell protein, and host cell DNA. After the Q-HP step, buffer exchange was performed as needed via Zeba gel filtration columns (Thermo) into PBS buffer (25 mM sodium phosphate, 150 mM sodium chloride, pH 7.4).

As shown in Table 3, the insulin-Fc fusions of the present technology synthesized in HEK293 cells exhibited an adequate titer. It has been determined that insulin-Fc fusion proteins of structures and compositions similar to the insulin-Fc fusion proteins of the current disclosure exhibiting protein-A purified titers in excess of 50 mg/L for transiently transfected HEK293 cells demonstrate higher, commercially viable CHO cell titers when the compounds are expressed using stably transfected CHO cells.

TABLE 3
Titer (mg/L) for Insulin-Fc Fusion Proteins
Manufactured in HEK293 Cells

| Sequence | Titer (mg/L) |
|---|---|
| SEQ ID NO: 1 | 80 |
| SEQ ID NO: 47 | 196 |
| SEQ ID NO: 22 | 107 |
| SEQ ID NO: 24 | 119 |
| SEQ ID NO: 26 | 106 |
| SEQ ID NO: 28 | 72 |
| SEQ ID NO: 30 | 99 |

### Example 2b: Purification of an Insulin-Fc Fusion Protein

Purification of an insulin-Fc fusion protein is performed as follows. Conditioned media supernatants containing the secreted Fc fusion protein are harvested from the transiently transfected HEK, stably transfected HEK, or stably transfected CHO production runs and are clarified by centrifugation. The supernatant containing the desired insulin-Fc fusion protein is run over a Protein A column, washed with various wash buffers including 0.15-0.50 M sodium chloride, and then eluted using a low pH solution. Afterwards, the eluted desired protein fractions are pooled, and buffer exchanged into 200 mM HEPES, 100 mM NaCl, 50 mM NaOAc, pH 7.0 buffer. A final filtration step is performed using a 0.2 μm membrane filter. The final protein concentration is calculated from the solution optical density at 280 nm. Further optional purification by ion-exchange chromatography (e.g., using an anion exchange bead resin or a cation exchange bead resin), gel filtration chromatography, or other methods is performed as necessary. In some examples, the insulin-Fc fusion is buffer exchanged via Zeba gel filtration columns (Thermo) into 50 mM sodium phosphate, pH 7.0 buffer and purified via Q-HP (Cytiva) ion exchange columns operating in flow through mode to remove molecular aggregates, host cell protein, and host cell DNA. After the Q-HP step, buffer exchange is performed as needed via Zeba gel filtration columns (Thermo) into PBS buffer (25 mM sodium phosphate, 150 mM sodium chloride, pH 7.4).

It is expected that the insulin-Fc fusion proteins of SEQ ID NO: 18 and SEQ ID NO: 20, synthesized in transiently transfected HEK293 cells, will exhibit protein-A purified titers in excess of 50 mg/L. It is expected that the insulin-Fc fusion proteins of SEQ ID NO: 1, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30 and SEQ ID NO; 47, when stably transfected in CHO-K1 GSN cells (LakePharma, Belmont, CA) will demonstrate stable pool titers of greater than 200 mg/mL and stable clone titers of greater than 500 mg/mL.

### Example 3a: Structure Confirmation by Non-Reducing and Reducing CE-SDS

Capillary electrophoresis sodium dodecyl sulfate (CE-SDS) analysis was performed in a LabChip® GXII (Perkin Elmer, Waltham, MA) on a solution of a purified insulin-Fc fusion protein dissolved in 200 mM HEPES, 100 mM NaCl, 50 mM NaOAc, pH 7.0 buffer, and the electropherogram was plotted. Under non-reducing conditions, the sample was run against known molecular weight (MW) protein standards, and the eluting peak represented the ‘apparent’ MW of the insulin-Fc fusion protein homodimer.

Under reducing conditions (e.g., using beta-mercaptoethanol to break disulfide bonds of the fusion protein), the apparent MW of the resulting insulin-Fc fusion protein monomer is compared against half the molecular weight of the insulin-Fc fusion protein homodimer as a way of determining that the structural purity of the insulin-Fc fusion protein is likely to be correct.

The non-reducing and reducing main peaks found via CE-SDS analysis for insulin-Fc fusion proteins of SEQ ID NO: 1 and SEQ ID NO: 47 synthesized in HEK293 cells are shown in Table 4, and 2× the apparent MW of the resulting insulin-Fc fusion protein monomer was compared to the molecular weight of the insulin-Fc fusion protein homodimer. The results in Table 4 illustrate that the structural purities of the insulin-Fc fusion proteins are likely to be correct.

TABLE 4
CE-SDS Non-Reducing and Reducing Main Peak for Insulin-Fc
Fusion Proteins Synthesized in HEK293 Cells

| Sequence | Non-reducing (kDa) Peak | Reducing (kDa) Peak | $\frac{{MW}_{homodimer}}{{MW}_{monomer}}$ |
|---|---|---|---|
| SEQ ID NO: 1 | 85.3 | 44.1 | 1.93 |
| SEQ ID NO: 47 | 77.7 | 36.9 | 2.11 |

### Example 3b: Structure Confirmation by Non-Reducing and Reducing CE-SDS

A capillary electrophoresis sodium dodecyl sulfate (CE-SDS) analysis is performed in a LabChip® GXII (Perkin Elmer, Waltham, MA) on a solution of a purified insulin-Fc fusion protein dissolved in 200 mM HEPES, 100 mM NaCl, 50 mM NaOAc, pH 7.0 buffer, and the electropherogram plotted. Under non-reducing conditions, the sample is run against known molecular weight (MW) protein standards, and the eluting peak represents the ‘apparent’ MW of the insulin-Fc fusion protein homodimer.

Under reducing conditions (e.g., using beta-mercaptoethanol to break disulfide bonds of the fusion protein), the apparent MW of the resulting insulin-Fc fusion protein monomer is compared against half the molecular weight of the insulin-Fc fusion protein homodimer as a way of determining that the structural purity of the insulin-Fc fusion protein is likely to be correct.

It is expected that the non-reducing and reducing main peaks that will be found via CE-SDS analysis for the insulin-Fc fusion proteins of SEQ ID NO: 16, SEQ ID NO: 18, SEQ ID NO: 20, SEQ ID NO: 22, SEQ ID NO: 24, SEQ ID NO: 26, SEQ ID NO: 28 and SEQ ID NO: 30, will illustrate that the structural purities of the insulin-Fc fusion proteins are likely to be correct.

### Example 4: Sequence Identification by LC-MS with Glycan Removal

To obtain an accurate estimate of the insulin-Fc mass via mass spectroscopy (MS), the sample is first treated to remove naturally occurring glycan that might interfere with the MS analysis. 100 μL of a 2.5 mg/mL insulin-Fc fusion protein dissolved in 200 mM HEPES, 100 mM NaCl, 50 mM NaOAc, pH 7.0 buffer solution is first buffer exchanged into 0.1 M Tris, pH 8.0 buffer containing 5 mM EDTA using a Zeba desalting column (ThermoFisher Scientific, Waltham, MA). 1.67 μL of PNGase F enzyme (Prozyme N-glycanase) is added to this solution in order to remove N-linked glycan present in the fusion protein, and the mixture is incubated at 37° C. overnight in an incubator. The sample is then analyzed via LC-MS (Novatia, Newtown, PA) resulting in a molecular mass of the molecule which corresponded to the desired homodimer without the glycan. This mass is then further corrected since the enzymatic process used to cleave glycan from asparagine also deaminates the asparagine side chain to form an aspartic acid, and in doing so the enzymatically treated homodimer gains 2 Da overall, corresponding to a mass of 1 Da for each chain present in the homodimer. Therefore, the actual molecular mass is the measured mass minus 2 Da to correct for the enzymatic modification of the insulin-Fc fusion protein structure in the analytical sample. The LC-MS expected molecular mass data, expected corrected mass data, and theoretical molecular masses (obtained via Expasy MW/pI tool) for exemplary insulin-Fc fusion proteins are shown in Table 5.

TABLE 5
Expected Molecular Mass by MS Compared to Theoretical

| | After PNGase treatment | | |
|---|---|---|---|
| | | | Desired |
| | | Measured | Homodimer |
| | | Mass, | Molecular |
| | Measured | Corrected | Mass |
| | Molecular | for N to D | (theoretical, |
| | Mass | transformation | from aa |
| Sequence | (Da) | (subtract 2 Da) | sequence, Da) |
| SEQ ID NO: 1 | 64406.7 ± 5 | 64404.7 ± 5 | 64404.7 |
| SEQ ID NO: 47 | 65219.0 ± 5 | 65217.0 ± 5 | 65217.0 |
| SEQ ID NO: 16 | 65003.3 ± 5 | 65001.3 ± 5 | 65001.3 |
| SEQ ID NO: 18 | 64350.6 ± 5 | 64348.6 ± 5 | 64348.6 |
| SEQ ID NO: 20 | 64432.8 ± 5 | 64430.8 ± 5 | 64430.8 |
| SEQ ID NO: 22 | 63077.2 ± 5 | 63075.2 ± 5 | 63075.2 |
| SEQ ID NO: 24 | 62961.1 ± 5 | 62959.1 ± 5 | 62959.1 |
| SEQ ID NO: 26 | 63133.2 ± 5 | 63131.2 ± 5 | 63131.2 |
| SEQ ID NO: 28 | 63121.3 ± 5 | 63119.3 ± 5 | 63119.3 |
| SEQ ID NO: 30 | 63177.3 ± 5 | 63175.3 ± 5 | 63175.3 |

### Example 5a: 00 Homodimer by Size-Exclusion Chromatography

Size-exclusion chromatography (SEC-HPLC) of insulin-Fc fusion proteins was carried out using a Waters 2795HT HPLC (Waters Corporation, Milford, MA) connected to a 2998 Photodiode array at a wavelength of 280 nm. 100 L or less of a sample containing an insulin-Fc fusion protein of interest was injected into a MAbPac SEC-1, 5 μm, 4×300 mm column (ThermoFisher Scientific, Waltham, MA) operating at a flow rate of 0.2 mL/min and with a mobile phase comprising 50 mM sodium phosphate, 300 mM NaCl, and 0.05% w/v sodium azide, pH 6.2. The MAbPac SEC-1 column operates on the principle of molecular size separation. Therefore, larger soluble insulin-Fc aggregates (e.g., multimers of insulin-Fc fusion protein homodimers) eluted at earlier retention times, and the non-aggregated homodimers eluted at later retention times. In separating the mixture of homodimers from aggregated multimeric homodimers via analytical SEC-HPLC, the purity of the insulin-Fc fusion protein solution in terms of the percentage of non-aggregated homodimer was ascertained. Table 6 shows the homodimer percentage of insulin-Fc fusion proteins manufactured in HEK293 cells.

TABLE 6
SE-HPLC Determined Percentage of Compound in Homodimer Form

| | | % Homodimer after |
|---|---|---|
| | | Protein A step and |
| | | Additional Q-HP ion |
| | % Homodimer | exchange column |
| | after Protein | step via the process |
| Sequence | A step of Example 2a | of Example 2a |
| SEQ ID NO: 1 | 87.0 | >98 |
| SEQ ID NO: 47 | 97.6 | >98 |

### Example 5b: % Homodimer by Size-Exclusion Chromatography

Size-exclusion chromatography (SEC-HPLC) of insulin-Fc fusion proteins is carried out using a Waters 2795HT HPLC (Waters Corporation, Milford, MA) connected to a 2998 Photodiode array at a wavelength of 280 nm. 100 L or less of a sample containing an insulin-Fc fusion protein of interest is injected into a MAbPac SEC-1, 5 μm, 4×300 mm column (ThermoFisher Scientific, Waltham, MA) operating at a flow rate of 0.2 mL/min and with a mobile phase comprising 50 mM sodium phosphate, 300 mM NaCl, and 0.05% w/v sodium azide, pH 6.2. The MAbPac SEC-1 column operates on the principle of molecular size separation. Therefore, larger soluble insulin-Fc aggregates (e.g., multimers of insulin-Fc fusion protein homodimers) elute at earlier retention times, and the non-aggregated homodimers elute at later retention times. In separating the mixture of homodimers from aggregated multimeric homodimers via analytical SEC-HPLC, the purity of the insulin-Fc fusion protein solution in terms of the percentage of non-aggregated homodimer is ascertained.

It is expected that the insulin-Fc fusion proteins of SEQ ID NO: 16, SEQ ID NO: 18, SEQ ID NO: 20, SEQ ID NO: 22, SEQ ID NO: 24, SEQ ID NO: 26, SEQ ID NO: 28 and SEQ ID NO: 30 will exhibit a homodimer percentage after Protein A step of Example 2b in excess of 80%. It is expected that the insulin-Fc fusion proteins of SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30 will exhibit a homodimer percentage after Protein A step and addition Q-HP ion exchange column step via the process of Example 2b in excess of 90%.

### Example 6a: In Vitro IM-9 Insulin Receptor (IR) Binding of Insulin-Fc Fusion Proteins at 4° C.

Human IM-9 cells (ATTC #CCL-159) that express human insulin receptor were cultured and maintained in complete RPMI 10% FBS medium at 70-80% confluency. Cultures of IM-9 cells were centrifuged at 250×g (~1000 rpm) for 10 min to pellet the cells. Cells were washed once with HBSS or PBS buffer, resuspended in cold FACS medium (HBSS/2 mM EDTA/0.1% Na-azide+2% horse serum) to a concentration of 1×107 cells/mL and kept on ice (4° C.) for 20-30 min in FACS buffer. Insulin-Fc proteins, insulins, or insulin analogs (e.g., test compounds) were diluted in FACS buffer in 1:4 serial dilutions as 2× concentrations (800 nM, 400 nM, 100 nM, 25 nM, 6.25 nM, 1.57 nM, 0.39 nM) in 1.2 mL tubes (approx. 60 μL volume of each dilution), and the solutions were kept on ice to reach 4° C. tubes until ready for pipetting.

Biotinylated-RHI was diluted in FACS staining medium as a 20× concentration at 10 μg/mL (final 0.5 μg/mL). 50 μL of each serially diluted test compound and 5 μL of 20× Biotin-RHI were added into each well of a V bottom microtiter plate, mixed, and placed on ice. 45 μL of IM-9 cell suspension was then added to each well by multichannel pipette, mixed again gently and incubated on ice for 30 min to allow competitive binding on the insulin receptor (IR) on IM-9 cells. Cells were then washed twice with 250 μL of ice-cold FACS wash buffer (HBSS/2 mM EDTA/0.1% Na-azide+0.5% horse serum) by centrifuging the V-bottom plate at 3000 rpm for 3 min and aspirating the supernatant. Cells were then resuspended in 50 L of FACS medium containing 1:200 diluted Streptavidin-PE(Life Technologies) for 20 min on ice. Cells were then washed once with 250 μL of ice-cold FACS buffer and finally fixed with 4% paraformaldehyde for 10 min.

Cells were then transferred to FACS tubes and analyzed on a Guava 8-HT flow cytometer (Millipore). Biotinylated-RHI binding to insulin receptor was quantitated by the median fluorescence intensity (MFI) of the cells on the FACS FL-2 channel and was measured for each concentration of the test compound. Control wells were labeled only with biotinylated-RHI and were used to calculate the % inhibition resulting from each test compound concentration. The percent (%) inhibition by test compounds of biotinylated-RHI binding on IM-9 cells was plotted against log concentrations of each test compound and IC₅₀ values were calculated using GraphPad Prism (GraphPad Software, La Jolla, CA) for each test compound. Lower IC₅₀ values of test compounds were reflective of stronger binding to insulin receptors (where the strength of the affinity or binding to the insulin receptor is relative to a reference insulin-Fc fusion protein). A control compound, such as unlabeled recombinant human insulin (RHI) was also used as an internal standard to generate an RHI IC₅₀ against which a given compound IC₅₀ could be ratioed (IC₅₀ (compound)/IC₅₀ (RHI)). Lower IC₅₀ ratios have more similar binding to RHI (stronger binding to insulin receptor), while higher IC₅₀ ratios have weaker binding to the insulin receptor relative to RHI. Inhibition of biotin labelled-insulin binding to IM-9 insulin receptor (IC₅₀; nM) of the test compound and inhibition of biotin labelled-insulin binding to IM-9 insulin receptor (IC₅₀; nM) of RHI were measured, and the IC₅₀ ratio of the test compound to RHI was determined.

It is noted that fusion proteins of the current technology exhibit weaker binding to the insulin receptor than RHI, as demonstrated by an IC₅₀ ratio of greater than 1. The term “strong binding to the insulin receptor” indicates a relative comparison of the insulin receptor binding ratio to RHI between different insulin-Fc fusion protein compounds. For example, an insulin-Fc fusion protein (referred to as ‘insulin-Fc fusion protein A’) with an IC₅₀ ratio of 20 means that the insulin-Fc fusion protein binds to the insulin receptor 20 times weaker than recombinant human insulin. However, a different insulin-Fc fusion protein with an IC₅₀ ratio of greater than 100 (referred to as ‘insulin-Fc fusion protein A’) binds the insulin receptor much less strongly than referred to as ‘insulin-Fc fusion protein A’. In this case, we may state that ‘insulin-Fc fusion protein A exhibits strong binding to the insulin receptor’ or that ‘insulin-Fc fusion protein A exhibits stronger binding to the insulin receptor than a reference insulin-Fc fusion protein’, where the reference insulin-Fc fusion protein may be SEQ ID NO: 47 which has an IC₅₀ ratio of 142. Alternatively, we may state that ‘insulin-Fc fusion protein A exhibits a low IC₅₀ ratio to RHI’. Such expressions may be interchangeable used to express that the exemplary insulin-Fc fusion proteins of the present technology exhibit relatively strong binding to the insulin receptor compared to other insulin-Fc fusion proteins.

The in vitro IM-9 insulin receptor binding for the insulin-Fc fusion protein of SEQ ID NO: 1 across two batches and of the insulin-Fc fusion protein of SEQ ID NO: 47 in a single batch is shown in Table 7. The results illustrate that the insulin-Fc fusion protein of SEQ ID NO:1 demonstrates high affinity for the insulin receptor compared to RHI and that the insulin-Fc fusion protein of SEQ ID NO: 47 has very low affinity for the insulin receptor.

TABLE 7
Inhibition of Biotin Labelled-insulin Binding to IM-9 Insulin Receptor and Associated
Compound Sequence Properties.

| | | | | AA at | AA at | |
|---|---|---|---|---|---|---|
| | | | | position 10 | position 8 of | |
| | | RHI | IC₅₀ Ratio | of insulin | insulin | |
| | Compound | IC₅₀ | (Compound/ | polypeptide | polypeptide | Linker AA |
| SEQ ID NO: | IC₅₀ (nM) | (nM) | RHI) | B chain | A chain | Sequence |
| SEQ ID NO: | 26 | 5 | 5 | D | H | GGGGAGGG |
| 1 | | | | | | G (SEQ ID |
| (Experiment | | | | | | NO: 11) |
| Batch #1) | | | | | | |
| SEQ ID NO: | 55 | 15 | 4 | D | H | GGGGAGGG |
| 1 | | | | | | G (SEQ ID |
| (Experiment | | | | | | NO: 11) |
| Batch #2) | | | | | | |
| SEQ ID NO: | 709 | 5 | 142 | H | T | GGGGGQGG |
| 47 | | | | | | GGQGGGGQ |
| | | | | | | GGGGG (SEQ |
| | | | | | | ID NO: 52) |

### Example 6b: In Vitro IM-9 Insulin Receptor (IR) Binding of Insulin-Fc Fusion Proteins at 4° C.

Human IM-9 cells (ATTC #CCL-159) that express human insulin receptor are cultured and maintained in complete RPMI 10% FBS medium at 70-80% confluency. Cultures of IM-9 cells are centrifuged at 250×g (~1000 rpm) for 10 min to pellet the cells. Cells are washed once with HLBSS or PBS buffer, resuspended in cold FACS medium (HBSS/2 mM EDTA/0.1% Na-azide+200 horse serum) to a concentration of 1×107 cells/mL and kept on ice (4° C.) for 20-30 min in FACS buffer. Insulin-Fc proteins, insulins, or insulin analogs (e.g., test compounds) are diluted in FACS buffer in 1:4 serial dilutions as 2× concentrations (800 nM, 400 nM, 100 nM, 25 nM, 6.25 nM, 1.57 nM, 0.39 nM) in 1.2 mL tubes (approx. 60 L volume of each dilution), and the solutions are kept on ice to reach 4° C. tubes until ready for pipetting.

Biotinylated-RHI is diluted in FACS staining medium as a 20× concentration at 10 μg/mL (final 0.5 μg/mL). 50 L of each serially diluted test compound and 5 μL of 20× Biotin-RHI are added into each well of a V bottom microtiter plate, mixed, and placed on ice. 45 μL of IM-9 cell suspension is then added to each well by multichannel pipette, mixed again gently and incubated on ice for 30 min to allow competitive binding on the insulin receptor (IR) on IM-9 cells. Cells are then washed twice with 250 μL of ice-cold FACS wash buffer (HBSS/2 mM EDTA/0.1% Na-azide+0.5% horse serum) by centrifuging the V-bottom plate at 3000 rpm for 3 min and aspirating the supernatant. Cells are then resuspended in 50 μL of FACS medium containing 1:200 diluted Streptavidin-PE(Life Technologies) for 20 min on ice. Cells are then washed once with 250 μL of ice-cold FACS buffer and finally fixed with 4% paraformaldehyde for 10 min.

Cells are then transferred to FACS tubes and analyzed on a Guava 8-HT flow cytometer (Millipore). Biotinylated-RHI binding to insulin receptor is quantitated by the median fluorescence intensity (MFI) of the cells on the FACS FL-2 channel and is measured for each concentration of the test compound. Control wells are labeled only with biotinylated-RHI and are used to calculate the % inhibition resulting from each test compound concentration. The percent (%) inhibition by test compounds of biotinylated-RHI binding on IM-9 cells is plotted against log concentrations of each test compound and IC₅₀ values are calculated using GraphPad Prism (GraphPad Software, La Jolla, CA) for each test compound. Lower IC₅₀ values of test compounds are reflective of stronger binding to insulin receptors. A control compound, such as unlabeled recombinant human insulin (RHI) is also used as an internal standard to generate an RHI IC₅₀ against which a given compound IC₅₀ could be ratioed (IC₅₀ (compound)/IC₅₀ (RHI)). Lower IC₅₀ ratios have more similar binding to RHI (stronger binding to insulin receptor), while higher IC₅₀ ratios have weaker binding to the insulin receptor relative to RHI. Inhibition of biotin labelled-insulin binding to IM-9 insulin receptor (IC₅₀; nM) of the test compound and inhibition of biotin labelled-insulin binding to IM-9 insulin receptor (IC₅₀; nM) of RHI are measured, and the IC₅₀ ratio of the test compound to RHI is determined. The meaning of the expression “stronger binding to insulin receptors” has been previously described and defined.

It is expected that the insulin-Fc fusion proteins of SEQ ID NO: 16, SEQ ID NO: 18, and SEQ ID NO: 20 will exhibit a ratio of (IC₅₀ (compound)/IC₅₀ (RHI)) less than 20, indicating stronger binding to the insulin receptor than a reference insulin-Fc fusion protein. It is expected that the insulin-Fc fusion proteins of SEQ ID NO: 22, SEQ ID NO: 24, SEQ ID NO: 26, SEQ ID NO: 28, and SEQ ID NO: 30 will exhibit a ratio of (IC₅₀ (compound)/IC₅₀ (RHI)) greater than 50, indicating weak binding to the insulin receptor.

### Example 7a: Downregulation of IR in HCT-116 Cells

HCT-116 cells were treated in vitro with either RHI, the insulin-Fc fusion protein of SEQ ID NO: 1 or the insulin-Fc fusion protein of SEQ ID NO: 47 at multiple concentrations (0.05-500 nM). Levels of tumor IR, phospho-IR+phospho-IGF1R (e.g., “phospho IR/IGF1R”), phospho-Akt (S473), pan Akt, and Beta (1) Actin expression were measured by Western blot. Tumors were lysed in RIPA buffer, electrophoresed on SDS-PAGE gels, transferred to PVDF membranes using a dry blotting system, and probed for the aforementioned proteins using antibodies from Cell Signaling at 1:1000 along with appropriate secondary antibodies known to those skilled in the art. Blots were imaged (cDigit blot scanner, Licor) and assessed using Image Studio software (Licor).

FIG. 4 provides Western blots that showed that after 72 hours of treatment, RHI caused observable downregulation of IR in HCT-116 cells. Unexpectedly, the insulin-Fc fusion protein of SEQ ID NO: 1 caused substantial downregulation of IR in HCT-116 cells at all concentrations tested. This is highlighted as 501 in FIG. 4. Treatments with the insulin-Fc fusion protein of SEQ ID NO: 1 caused downregulation of IR even at very low concentrations. Furthermore, the insulin-Fc fusion protein of SEQ ID NO: 1 induced lower levels of Akt phosphorylation than RHI at every concentration tested. This is an important finding, as blocking Akt signaling through the use of current cancer therapies has been correlated with slower tumor growth, as Akt signaling is involved in cell proliferation and growth. Unexpectedly, FIG. 4 illustrates that the insulin-Fc fusion protein of SEQ ID NO: 47 did not cause downregulation of IR at any concentration tested. This is highlighted as 503 in FIG. 4. The insulin-Fc fusion protein of SEQ ID NO: 47 induce lower levels of Akt ph phosphorylation than RHI at any concentration tested.

The Western blots in FIG. 4 additionally show that the insulin-Fc fusion protein of SEQ ID NO: 1 demonstrated measurable Phospho IR/IGF1R despite downregulating the IR. This is highlighted as 502 in FIG. 4. This illustrates that treatment with the insulin-Fc fusion protein of SEQ ID NO: 1 unexpectedly caused substantial downregulation of IR while at the same time activating the signal pathway to the insulin receptor (IR), enabling uptake of insulin, and lowering the risk of hyperglycemia that has been associated with downregulation of IR in other therapeutic approaches (e.g., without limitation, anti-IR antibodies), or preventing hyperglycemia altogether. FIG. 4 illustrates that the insulin-Fc fusion protein of SEQ ID NO: 47 did not demonstrate measurable Phospho IR/IGF1R.

### Example 7b: Downregulation of IR in HCT-116 Cells

HCT-116 cells are treated in vitro with either RHI, or the insulin-Fc fusion proteins of SEQ ID NO: 16, SEQ ID NO: 18, SEQ ID NO: 20, SEQ ID NO: 22, SEQ ID NO: 24, SEQ ID NO: 26, SEQ ID NO: 28, or SEQ ID: 30 at multiple concentrations (0.05-500 nM). Levels of tumor IR, phospho-IR, phospho-IGF1R, phospho-Akt (S473), pan Akt, and Beta (1) Actin expression are measured by Western blot. Tumors are lysed in RIPA buffer, electrophoresed on SDS-PAGE gels, transferred to PVDF membranes using a dry blotting system, and probed for the aforementioned proteins using antibodies from Cell Signaling at 1:1000 along with appropriate secondary antibodies known to those skilled in the art. Blots are imaged (cDigit blot scanner, Licor) and assessed using Image Studio software (Licor).

It is expected that Western blots created after 72 hours of treatment will demonstrate that the insulin-Fc fusion proteins of SEQ ID NO: 16, SEQ ID NO: 18 and SEQ ID NO: 20 will cause substantial downregulation of IR in HCT-116 cells at all concentrations tested, and even at very low concentrations. It is further expected that the insulin-Fc fusion proteins of SEQ ID NO: 16, SEQ ID NO: 18 and SEQ ID NO: 20 will induce lower levels of Akt phosphorylation than RHI at every concentration tested. It is additionally expected that the insulin-Fc fusion proteins of SEQ ID NO: 16, SEQ ID NO: 18 and SEQ ID NO: 20 will demonstrate measurable Phospho IR/IGF1R despite downregulating the IR. Therefore it is expected that treatment with the insulin-Fc fusion proteins of SEQ ID NO: 16, SEQ ID NO: 18 and SEQ ID NO: 20 will unexpectedly cause substantial downregulation of IR while at the same time activating the signal pathway to the insulin receptor (IR), enabling uptake of insulin, and lowering the risk of hyperglycemia that has been associated with downregulation of IR in other therapeutic approaches (e.g., without limitation, anti-IR antibodies), or preventing hyperglycemia altogether.

Western blots created after 72 hours of treatment with the insulin-Fc fusion proteins of SEQ ID NO: 22, SEQ ID NO: 24, SEQ ID NO: 26, SEQ ID NO: 28, and SEQ ID NO: 30 are not expected to demonstrate measurable downregulation of the insulin receptor, and in a therapeutic setting would be predicted to allow a patient's own endogenous insulin to bind abundant insulin receptor (IR) resulting in tumor proliferation and growth, which would be undesirable.

### Example 8a: In Vivo Pharmacodynamics (PD) after Single Administration of Fusion Protein in Mice

A bioactive fusion protein construct of the insulin-Fc fusion protein of SEQ ID NO: 1 was synthesized according to Example 1a and assessed for its effects on fasting blood glucose levels as follows. Naïve, non-fasted nude mice were used. On Day 0, the mice received a single injection of a pharmaceutical composition containing a fusion protein homodimer of the insulin-Fc fusion protein of SEQ ID NO: 1 in a solution of 50 mM sodium hydrogen phosphate, 150 mM sodium chloride, and 0.02% v/v Tween-80 at pH 7.5, at a dose of 6 nmol/kg (equivalent to 0.39 mg Fc fusion protein/kg or 1.9 U/kg insulin equivalent on molar basis). Blood was collected immediately prior to injection and at 15, 30, 45, 60, 120, 240, 360, and 480 minutes and at 1, 2, 3, 4, 5, 6, 7, and 8 days post injection. On Day 0, blood was collected from a suitable vein immediately prior to injection as well as for the rest of the post-treatment timepoints.

For each time point, a minimum of 0.1 mL of whole blood was collected. A glucose level reading was immediately determined using a glucose meter (ACCU-CHEK® Aviva Plus), which requires approximately one drop of blood. Average % fasting blood glucose levels (% FBGL) from Day 0 to Day 8 were plotted in FIG. 5, which allows the bioactivity of a fusion protein to be determined. FIG. 5 demonstrates that the insulin-Fc fusion protein of SEQ ID NO: 1 can lower blood glucose for a significant period of time on a single dose.

### Example 8b: In Vivo Pharmacodynamics (PD) after Single Administration of Fusion Protein in Mice

Insulin-Fc fusion protein constructs of SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30 and SEQ ID NO: 47 are synthesized according to Example 1b, or Example 1c and are assessed for their effects on fasting blood glucose levels as follows. Naïve, non-fasted nude mice are used. On Day 0, the mice receive a single injection of a pharmaceutical composition containing an insulin-Fc fusion protein homodimer of SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30 or SEQ ID NO: 47 in a solution of 50 mM sodium hydrogen phosphate, 150 mM sodium chloride, and 0.02% v/v Tween-80 at pH 7.5, at a dose of 6 nmol/kg (equivalent to 0.39 mg Fc fusion protein/kg or 1.9 U/kg insulin equivalent on molar basis). Blood is collected immediately prior to injection and at 15, 30, 45, 60, 120, 240, 360, and 480 minutes and at 1, 2, 3, 4, 5, 6, 7, and 8 days post injection. On Day 0, blood is collected from a suitable vein immediately prior to injection as well as for the rest of the post-treatment timepoints.

For each time point, a minimum of 0.1 mL of whole blood is collected. A glucose level reading is immediately determined using a glucose meter (ACCU-CHEK® Aviva Plus), which requires approximately one drop of blood. Average % fasting blood glucose levels (% FBGL) from Day 0 to Day 8 are plotted, which allows the bioactivity of a fusion protein to be determined.

### Example 9a: Tumor Volume in HCT-116 Xenograft Models in Nude Mice

HCT-116 cells were cultured under aseptic conditions at 37° C. with 5% CO2 in logarithmic growth phase. On the day of the inoculations, the cells were harvested, washed in PBS, and resuspended at the appropriate concentration in a serum-free medium:matrigel (1:1 vol:vol) mixture. Inoculations were carried out in conscious naïve, nude mice (n=60 females) while being manually restrained. Mice were injected subcutaneously (SC) on their dorsal right flank with 2×106 cells using a 28G needle in 200 μL volume. The injection areas were monitored until the tumors were visible/palpable. Once palpable, calipers were used for tumor measurements twice a week. The greatest longitudinal diameter (length) and the greatest transverse diameter (width) was used to determine tumor volume according to the equation:

$$Tumor volume = \frac{( length \times {width}^{2} )}{2} .$$

Once the tumors reached a volume between 100 and 300 mm³, mice bearing HCT-116 tumors were randomized into four groups according to Table 8.

TABLE 8
Test Groups for HCT-116 Bearing Nude Mice

| | Fasted 8-10 | |
|---|---|---|
| | hours per day | Unfasted |
| Vehicle | fasted controls | unfasted controls |
| Insulin-Fc fusion protein | fasted treated | unfasted treated |

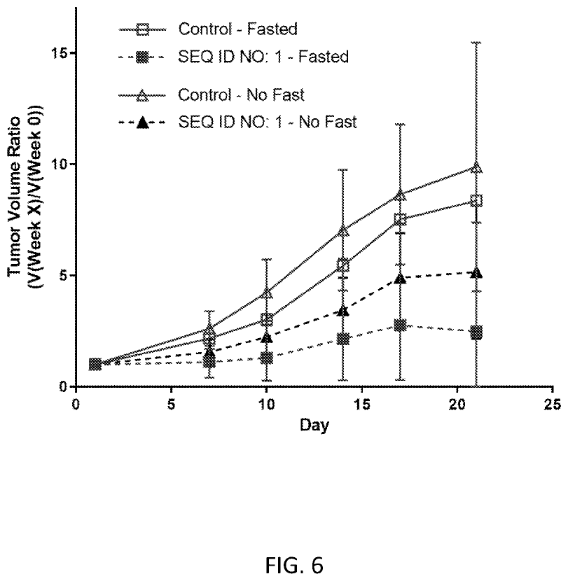

Tumor dimensions were measured before the testing using calipers and the tumor volume approximated using the formula given above. Tumor volume ratio (TVR) is defined as the ratio of the volume of the tumor at day X over the volume of the tumor at day 0. The mice were injected subcutaneously with either vehicle, 150 μg/kg of SEQ ID NO: 1, or 2.5 U/kg of conventional NPH insulin every day for 6 consecutive days followed by 1 day with no injection for a total of 3 weeks and subject to between 8 and 10 hours a day of fasting. As controls, parallel groups of treated and untreated mice were left unfasted. Tumor volume was measured before and after the testing and the measurements are shown in Table 9. Tumor volume ratio (TVR) is shown in FIG. 6.

TABLE 9
HCT-116 Tumor Volume Ratio (TVR), SEQ ID NO: 1 Treated
and Controls, Fasted and Unfasted and Fasted NPH

| | Volume | Volume | Comparison Result |
|---|---|---|---|
| | Before | After | (treated vs. controls) |
| Fasted treated | 158.3 | 505.6 | 62 ± 19% lower TVR |
| (SEQ ID NO: 1) | | | for SEQ ID NO: 1 |
| Fasted controls | 157.6 | 1279.6 | treated group vs. fasted |
| | | | controls, p < 0.02 |
| Fasted NPH | 157.2 | 1073.9 | Change not significant |
| | | | against fasted controls |
| Unfasted treated | 163.8 | 887.4 | 48% ± 14% lower TVR for |
| (SEQ ID NO: 1) | | | SEQ ID NO: 1 |
| Unfasted controls | 154.9 | 1542.7 | treated vs. unfasted |
| | | | controls, p < 0.02 |

### Example 9b: Tumor Volume in HCT-116 Xenograft Models in Nude Mice

HCT-116 cells are cultured under aseptic conditions at 37° C. with 50 CO2 in logarithmic growth phase. On the day of the inoculations, the cells are harvested, washed in PBS, and resuspended at the appropriate concentration in a serum-free medium:matrigel (1:1 vol:vol) mixture. Inoculations are carried out in conscious naïve, nude mice (n=60 females) while being manually restrained. Mice are injected subcutaneously (SC) on their dorsal right flank with 2×106 cells using a 28G needle in 200 μL volume. The injection areas are monitored until the tumors are visible/palpable. Once palpable, calipers are used for tumor measurements twice a week. The greatest longitudinal diameter (length) and the greatest transverse diameter (width) is used to determine tumor volume according to the equation:

$$Tumor volume = \frac{( length \times {width}^{2} )}{2} .$$

Once the tumors reach a volume between 100 and 300 mm³, mice bearing HCT-116 tumors are randomized into four groups according to Table 10.

TABLE 10
Test Groups for HCT-116 Bearing nude Mice

| | Fasted 8-10 hours per day | Unfasted |
|---|---|---|
| Vehicle | fasted controls | unfasted controls |
| Insulin-Fc fusion protein | fasted treated | unfasted treated |

Tumor dimensions are measured before the testing using calipers and the tumor volume approximated using the formula given above. Tumor volume ratio (TVR) is defined as the ratio of the volume of the tumor at day X over the volume of the tumor at day 0. The mice are injected subcutaneously with either vehicle, 150 μg/kg of the insulin-Fc fusion proteins of SEQ ID NO: 47, SEQ ID NO: 16, SEQ ID NO: 18, SEQ ID NO: 20, SEQ ID NO: 22, SEQ ID NO: 24, SEQ ID NO: 26, SEQ ID NO: 28 or SEQ ID NO: 30, or 2.5 U/kg of conventional NPH insulin every day for 6 consecutive days followed by 1 day with no injection for a total of 3 weeks and subject to between 8 and 10 hours a day of fasting. As controls, parallel groups of treated and untreated mice are left unfasted. Tumor volume is measured before and after the testing. It is expected that the tumor volume ratio (TVR) for mice injected with the insulin-Fc fusion proteins of SEQ ID NO: 16, SEQ ID NO: 18, and SEQ ID NO: 20 compared to the fasted control group will be at least 30% lower. It is expected that the tumor volume ratio (TVR) for mice injected with the insulin-Fc fusion proteins of SEQ ID NO: 16, SEQ ID NO: 18, and SEQ ID NO: 20, compared to the no fasting control group will be at least 40% lower. It is expected that the tumor volume ratio (TVR) for mice injected with the insulin-Fc fusion proteins of SEQ ID NO: 22, SEQ ID NO: 24, SEQ ID NO: 26, SEQ ID NO: 28, SEQ ID NO: 30, and SEQ ID NO: 47 will remain similar to the fasting control group and the no fasting control group.

### Example 10a: Tumor Volume in Metastatic Human Melanoma Cell Line (WM266.4)

WM266.4 cells were cultured under aseptic conditions at 37° C. with 5% CO₂ in logarithmic growth phase. On the day of the inoculations, the cells were harvested, washed in PBS, and resuspended at the appropriate concentration in a serum-free medium:matrigel (1:1 vol:vol) mixture. Inoculations were carried out in conscious Naïve, nude mice (n=60 females) while being manually restrained. Mice were injected subcutaneously (SC) on their dorsal right flank with 2×10⁶ cells using a 28G needle in 200 μl volume. The injection areas were monitored until the tumors were visible/palpable. Once palpable, calipers were used for tumor measurements twice a week. The greatest longitudinal diameter (length) and the greatest transverse diameter (width) was used to determine tumor volume according to the equation:

$$Tumor volume = \frac{( length \times {width}^{2} )}{2} .$$

Once the tumors reach a volume between 100 and 300 mm³, mice bearing WM266.4 tumors were randomized into four groups according to Table 11.

TABLE 11
Test Groups for WM266.4 Bearing Nude Mice

| | Fasted 8-10 hours per day | Unfasted |
|---|---|---|
| Vehicle | fasted controls | unfasted controls |
| Insulin-Fc fusion protein | fasted treated | unfasted treated |

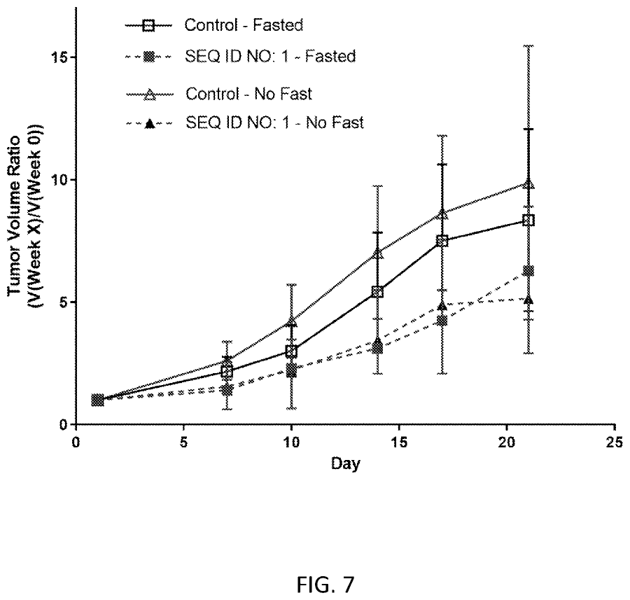

Tumor dimensions were measured before the testing using calipers and the tumor volume approximated using the formula given above. Tumor volume ratio (TVR) is defined as the ratio of the volume of the tumor at day X over the volume of the tumor at day 0. The mice were injected subcutaneously with either vehicle or 150 μg/kg of the insulin-Fc fusion protein of SEQ ID NO: 1, every day for 6 consecutive days followed by 1 day with no injection for a total of 3 weeks and were subject to between 8 and 10 hours a day of fasting. As controls, parallel groups of treated and untreated mice were left unfasted. Tumor volume was measured before and after the testing and the measurements are shown in Table 12. Tumor volume ratio (TVR) is shown in FIG. 7.

TABLE 12
WM266.4 Tumor Volume Ratio, SEQ ID NO: 1 Treated
and Controls, Fasted and Unfasted

| | Volume | Volume | Comparison Result |
|---|---|---|---|
| | Before | After | (treated vs. controls) |
| Fasted treated (SEQ | 148.1 | 1156.6 | 62 ± 19% lower TVR for SEQ |
| ID NO: 1) | | | ID NO: 1 treated vs. fasted |
| Fasted controls | 146.3 | 1685.9 | controls, p < 0.02 |
| Unfasted treated | 149.2 | 981.9 | 48% ± 14% lower TVR for |
| (SEQ ID NO: 1) | | | SEQ ID NO: 1 treated vs. |
| Unfasted controls | 146.3 | 1685.9 | unfasted controls, p < 0.02 |

### Example 10b: Tumor Volume in Metastatic Human Melanoma Cell Line (WM266.4)

WM266.4 cells are cultured under aseptic conditions at 37° C. with 500 CO₂ in logarithmic growth phase. On the day of the inoculations, the cells are harvested, washed in PBS, and resuspended at the appropriate concentration in a serum-free medium:matrigel (1:1 vol:vol) mixture. Inoculations are carried out in conscious Naïve, nude mice (n=60 females) while being manually restrained. Mice are injected subcutaneously (SC) on their dorsal right flank with 2×10⁶ cells using a 28G needle in 200 μl volume. The injection areas are monitored until the tumors were visible/palpable. Once palpable, calipers are used for tumor measurements twice a week. The greatest longitudinal diameter (length) and the greatest transverse diameter (width) is used to determine tumor volume according to the equation:

$$Tumor volume = \frac{( length \times {width}^{2} )}{2} .$$

Once the tumors reach a volume between 100 and 300 mm³, mice bearing WM266.4 tumors are randomized into four groups according to Table 13.

TABLE 13
Test Groups for WM266.4 Bearing Nude Mice

| | Fasted 8-10 hours per day | Unfasted |
|---|---|---|
| Vehicle | fasted controls | unfasted controls |
| Insulin-Fc fusion protein | fasted treated | unfasted treated |

Tumor dimensions are measured before the testing using calipers and the tumor volume approximated using the formula given above. Tumor volume ratio (TVR) is defined as the ratio of the volume of the tumor at day X over the volume of the tumor at day 0. The mice are injected subcutaneously with either vehicle, 150 μg/kg of the insulin-Fc fusion proteins of SEQ ID NO: 47, SEQ ID NO: 16, SEQ ID NO: 18, SEQ ID NO: 20, SEQ ID NO: 22, SEQ ID NO: 24, SEQ ID NO: 26, SEQ ID NO: 28 or SEQ ID NO: 30, every day for 6 consecutive days followed by 1 day with no injection for a total of 3 weeks and are subject to between 8 and 10 hours a day of fasting. As controls, parallel groups of treated and untreated mice are left unfasted. Tumor volume is measured before and after the testing. It is expected that the tumor volume ratio (TVR) for mice injected with the insulin-Fc fusion proteins of SEQ ID NO: 16, SEQ ID NO: 18, and SEQ ID NO: 20, compared to the fasted control group will be at least 50% lower. It is expected that the tumor volume ratio (TVR) for mice injected with the insulin-Fc fusion proteins of SEQ ID NO: 16, SEQ ID NO: 18, or SEQ ID NO: 20 compared to the no fasting control group will be at least 40% lower. It is further expected that the SEQ ID NO: 18 and SEQ ID NO: 20 will show greater efficacy in reducing tumor volume ratio compared to SEQ ID NO: 1 or SEQ ID NO: 16 due to being less immunogenic due to decreased Fc(gamma)R binding (measured as described in Example 14) and decreased C1q binding (measured as described in Example 15). It is expected that the tumor volume ratio (TVR) for mice injected with the insulin-Fc fusion proteins of SEQ ID NO: 22, SEQ ID NO: 24, SEQ ID NO: 26, SEQ ID NO: 28, SEQ ID NO: 30 and SEQ ID NO: 47 will remain similar to the fasting control group and the no fasting control group.

### Example 11: Tumor Volume in Human Breast Cancer Cell Line (MCF-7L) and Tamoxifen-Resistant Breast Cancer Cell Line (MCF-7L TamR) In Vivo without Fasting

MCF-7L or MCF-7L TamR (TamR=tamoxifen resistant) cells differ from each other in that MCF-7L cells express significant IR and IGF1R on their cell surfaces, whereas MCF-7L TamR cells express very low levels of IGF1R and significant levels of IR on their cell surfaces as measured by Western Blot techniques. These cells are cultured under aseptic conditions at 37° C. with 5% CO₂ in logarithmic growth phase. On the day of the inoculations, the cells are harvested, washed in PBS, and resuspended at the appropriate concentration in a serum-free medium. Mice are implanted bilaterally into the second mammary fat pads of female nude mice, using 1×10⁶ cells per implantation. Mice are implanted on both the left and right sides. The injection areas are monitored until the tumors were visible/palpable. Once palpable, calipers are used for tumor measurements twice a week. The greatest longitudinal diameter (length) and the greatest transverse diameter (width) are used to determine tumor volume according to the equation:

$$Tumor volume = \frac{( length \times {width}^{2} )}{2} .$$

Once the tumors reach a volume between 100 and 300 mm³, mice bearing MCF-7L tumors are randomized into groups according to Table 14.

TABLE 14
Test Groups for MCF-7L Bearing Nude Mice

| | Fasted 8-10 hours per day | Unfasted |
|---|---|---|
| Vehicle | fasted controls | unfasted controls |
| Insulin-Fc fusion protein | fasted treated | unfasted treated |

Tumor dimensions are measured before the testing using calipers and the tumor volume approximated using the formula given above. Tumor volume ratio (TVR) is defined as the ratio of the volume of the tumor at week X over the volume of the tumor at week 0. The mice are injected subcutaneously with either vehicle or 100-200 μg/kg of the insulin-Fc fusion proteins of SEQ ID NO: 1, SEQ ID NO: 47, SEQ ID NO: 16, SEQ ID NO: 18, SEQ ID NO: 20, SEQ ID NO: 22, SEQ ID NO: 24, SEQ ID NO: 26 SEQ ID NO: 28 or SEQ ID NO: 30 3× per week for up to 8 weeks. Vehicle and treated group tumor volume is measured before and after the testing and the expected measurements are shown in Table 15, with treatments using the insulin-Fc fusion proteins of SEQ ID NO: 1, SEQ ID NO: 16, SEQ ID NO: 18, and SEQ ID NO: 20 expected to show significant effects both alone and in combination with tamoxifen treatment. It is further expected that the insulin-Fc fusion proteins of SEQ ID NO: 18 and SEQ ID NO: 20 will show greater efficacy in reducing tumor volume ratio compared to the insulin-Fc fusion proteins of SEQ ID NO: 1 or SEQ ID NO: 16 due to being less immunogenic due to decreased Fc(gamma)R binding (measured as described in Example 14) and decreased C1q binding (measured as described in Example 15).

The insulin-Fc fusion proteins of SEQ ID NO: 1, SEQ ID NO. 16, SEQ ID NO: 18, and SEQ ID NO: 20 are referred to as Group A in Table 15, and the insulin-Fc fusion proteins of SEQ ID NO: 22, SEQ ID NO: 24, SEQ TD NO: 26, SEQ ID NO: 28, SEQ ID NO: 30 and SEQ TD NO: 47, are referred to as Group B in Table 15.

TABLE 15
Test Groups and Expected Outcomes for
MCF-7L Bearing Nude Mice Using
SEQ ID Nos. 1 and 47, Tamoxifen, or Vehicle; no Fasting.
Vehicle is PBS or Peanut Oil. Tam = Tamoxifen

| | | | Expected | Expected |
|---|---|---|---|---|
| | | | Result | Result |
| | | SEQ | for TVR | for TVR |
| | | ID | (Week8/ | (Week8/ |
| Treat- | | NO/ | Week0)# | Week0)# |
| ment | Treatment | Dose | as compared to | as compared to |
| ID | Combination | (μg/kg) | Vehicle/Vehicle | Vehicle/Tam |
| A | Vehicle (PBS)/ | N/A | 1 | 0 |
| | Vehicle (peanut | | | |
| | oil) | | | |
| B | Vehicle (PBS)/ | N/A | 3 | 4 |
| | Tam | | | |
| C | Group A/Vehicle | 100 | 2 | 0 |
| D | Group A/Tam | 100 | 3 | 5 |
| E | Group A/Vehicle | 200 | 2 | 0 |
| F | Group A/Tam | 200 | 3 | 6 |
| G | Group B/Vehicle | 200 | * | * |
| H | Group B/Tam | 200 | * | * |

#: 0 = increased TVR versus vehicle control,
1 = no change versus Vehicle/Vehicle;
2 = small TVR reduction versus Vehicle/Vehicle;
3 = significant TVR reduction versus Vehicle/Vehicle;
4 = no change versus Vehicle/Tam,
5 = small TVR reduction versus Vehicle/Tam,
6 = significant TVR reduction versus Vehicle/Tam.
* = mice expected to be culled from study due to significant hypoglycemia; not able to complete study.

MCF-7L TamR tumors are also studied in vivo by implanting MCF-7L TamR cells in vivo into female nude mice as described above. Once the tumors reach a volume between 100 and 300 mm³, mice bearing MCF-7L TamR tumors are randomized into groups according to Table 14 in a separate study. Tumor dimensions are measured before the testing using calipers and the tumor volume approximated using the formula given above. Tumor volume ratio (TVR) is defined as the ratio of the volume of the tumor at week X over the volume of the tumor at week 0. The mice are injected subcutaneously with either vehicle or 100-200 μg/kg of the insulin-Fc fusion protein of SEQ ID NO: 1, SEQ ID NO: 47, SEQ ID NO: 16, SEQ ID NO: 18, SEQ ID NO: 20, SEQ ID NO: 22, SEQ ID NO: 24, SEQ ID NO: 26, SEQ ID NO: 28 or SEQ ID NO: 30 3× per week for up to 8 weeks. Vehicle and treated group tumor volume is measured before and after the testing and the expected measurements are shown in Table 16, with the insulin-Fc fusion proteins of SEQ ID NO: 1, SEQ ID NO: 16, SEQ ID NO 18, and SEQ ID NO: 20 treatment expected to show significant effects as compared to tamoxifen treatment alone. It is further expected that the insulin-Fc fusion proteins of SEQ ID NO: 18 and SEQ ID NO: 20 will show greater efficacy in reducing tumor volume ratio compared to the insulin-Fc fusion proteins of SEQ ID NO: 1 or SEQ ID NO: 16 due to being less immunogenic due to decreased Fc(gamma)R binding (measured as described in Example 14) and decreased C1q binding (measured as described in Example 15).

This is expected since MCF-7L TamR cells are tamoxifen resistant and therefore tamoxifen treatment alone should have little to no impact on tumor growth, whereas the insulin-Fc fusion protein of SEQ ID NO: 1, SEQ ID NO: 16, SEQ ID NO: 18 or SEQ ID NO: 20 treatment, particularly at higher doses is expected to downregulate the insulin receptor on MCF-7L TamR cells resulting in slower tumor growth and lower TVR as compared to tamoxifen treatment alone.

TABLE 16
Test Groups and Expected Outcomes for MCF-7L TamR Bearing
Nude Mice Using SEQ ID NOs. 1 and 47, Tamoxifen, or
Vehicle; no Fasting. Vehicle is PBS or Peanut Oil. Tam = Tamoxifen

| | | SEQ ID | Expected Result for TVR |
|---|---|---|---|
| | | NO. | (Week8/Week0)# |
| Treatment | Treatment | Dose | as compared to |
| ID | Combination | (ug/kg) | Vehicle/Vehicle |
| I | Vehicle/Tam | N/A | 4 |
| J | Group A/Tam | 100 | 5 |
| K | Group A/Tam | 200 | 6 |

#: 0 = increased TVR versus vehicle control (undesirable),
4 = no change versus Vehicle/Tam,
5 = small TVR reduction versus Vehicle/Tam,
6 = significant TVR reduction versus Vehicle/Tam.

### Example 12: Assay Protocol for Measuring Anti-Drug Antibodies in Human Serum

Maxisorp ELISA Plates (Nunc) are coated with the insulin-Fc fusion protein of interest diluted in coating buffer (pH=9.6 Carbonate-Biocarbonate buffer) at 10 μg/mL overnight at 4° C. for measuring ADAs against the test compound. For measuring ADAs against the insulin portion of the insulin-Fc fusion protein containing an Fc fragment of human IgG origin, plates are coated with purified insulin at 30 μg/mL in coating buffer. Plates are then washed 5× with PBST (PBS+0.05% Tween 20) and blocked for at least 1 hour (or overnight) with SuperBlock blocking solution (ThermoFisher, Waltham M A). For calculating the ADAs in human IgG units, strips are directly coated with 1:2 serial dilutions of human IgG (Jackson Immunoresearch Laboratories, West Grove PA) in pH=9.6 Carb-Biocarb coating buffer at concentrations between 300-4.69 ng/ml overnight at 4° C. and used to create a 7-point pseudo-standard curve. The standards strip plates are also washed and blocked with SuperBlock blocking solution for at least 1 hour (or overnight).

Test serum samples are diluted to greater than or equal to 1:100 (typically tested as 1:200) in PBST/SB/20% HS sample dilution buffer (PBS+0.1% Tween 20+10% SuperBlock+20% horse serum) and added to the insulin-Fc fusion protein coated (or RHI coated) strips at 100 μL/well in duplicate. Duplicate strips of human IgG coated standard strips are also added to each plate and filled with PBST/SB (PBS+0.1% Tween 20+10% SuperBlock) buffer at 100 μL/well. Plates are incubated for 1 hour at RT and then washed 5× with PBST. For detection of ADAs, HRP-conjugated Goat anti-feline IgG F(ab′)2 (anti-feline IgG F(ab′)2 reagent is cross-reacted to human antibodies; Jackson Immunoresearch Laboratories, West Grove PA), which is diluted in PBST/SB to 1:10000 and added at 100 μL/well to both sample and standard wells and incubated for 45 minutes at RT in dark. Plates are washed 5× with PBST and then one time with deionized water and then developed by adding 100 μL/well TMB substrate (Invitrogen, ThermoFisher Scientific, Waltham MA) for 15-20 minutes at room temperature in the dark. Color development is then stopped by addition of 100 μL/well of ELISA Stop Solution (Boston Bioproducts) and the absorbance is read at 450 nm using a SpectraMax plate reader within 30 minutes. The anti-drug antibody concentration is determined by interpolating the OD values in the 4-PL pseudo-standard curve using SoftMax Pro Software (Molecular Devices, San Jose CA).

To demonstrate the specificity of the detected ADAs, an “inhibition” assay is carried out. In the drug inhibition ADA assay, serum samples are diluted 1:100 in PBST/SB/20% HS buffer and mixed with an equal volume of 300 μg/mL of the relevant therapeutic compound (final sample dilution at 1:200 and final inhibitory compound at 150 μg/mL) and incubated for 30-40 minutes at room temperature to allow anti-drug antibodies to bind the free inhibitor (i.e., the therapeutic compound). After pre-incubation, the samples are added to insulin-Fc fusion protein coated (or RHI coated) strips at 100 μL/well in duplicate. Samples diluted 1:200 in PBST/SB/20% HS buffer without the inhibitory compound are also tested in the sample plates along with duplicate strips of human IgG coated standards. Remaining steps of the assay procedure are carried out as described above. The ADAs measured in the drug-inhibited wells are matched with the non-inhibited ADA concentrations to assess the specificity of the ADAs. If significant inhibition of ADA signals is observed in the drug-inhibited wells, this means the ADAs are specific to the therapeutic compound.

### Example 13: Assay Procedure for Immunogenic Epitope Identification

Maxisorp ELISA microplates (Nunc) are coated with a library of insulin-Fc fusion protein homodimer compounds with known amino acid sequences, and the coated plates are blocked in a similar manner as described in the anti-drug antibody ELISA assay Example 12, except that each compound in the library is coated on a separate individual strip of ELISA microplate wells. The compounds in the library comprise a range of insulin-Fc fusion proteins with different insulin polypeptide amino acid compositions, including various B-chain, C-chain, and A-chain amino acid mutations, different linker compositions, and different Fc fragment compositions, including some of human origin. Separately, some plate strip wells are directly coated with 1:2 serial dilutions of human IgG (Jackson Immunoresearch Laboratories, West Grove PA) for calculating the anti-drug antibodies (ADA) in human IgG units, respectively, as described in Example 12.

Serum obtained from individual patients receiving repeated doses of an insulin-Fc fusion protein is first screened on the anti-drug antibody ELISA assay (Example 12). Serum samples demonstrating moderate or high positivity (e.g., moderate or high titers of antibodies) on the assay of Example 12 are serially diluted (1:200 to 1:8000) in PBST/SB/20% HS sample dilution buffer (PBS+0.1% Tween 20+10% SuperBlock+20% horse serum) and added to the plates coated with the library of insulin-Fc fusion protein compounds for 1 hour at RT. Following incubation, the plates are washed 5 times with PBST. For detection of human antibodies capable of cross-reacting to the coated compound library, HRP conjugated goat anti-feline IgG F(ab′)2 (Jackson Immunoresearch Laboratories, West Grove PA), which is cross-reactive to canine IgGs, is diluted in PBST/SB to 1:10000 and added at 100 μL/well to both sample and standard wells and incubated for 45 min at RT in the dark. Plates are washed 5 times with PBST, once with deionized water, and developed by the adding 100 μL/well TMB substrate (Invitrogen, ThermoFisher Scientific, Waltham MA) for 15-20 min at RT in the dark. Color development is then stopped by addition of 100 μL/well of ELISA Stop Solution (Boston Bioproducts, Ashland MA) and absorbance is read at 450 nm using a SpectraMax plate reader within 30 min. Anti-compound cross-reactive antibody concentrations present in the serum samples are determined by interpolating the OD values in the 4-PL pseudo-standard curve against the directly coated human IgG antibody controls using SoftMax Pro Software (Molecular Devices, San Jose CA).

By correlating the resulting antibody concentrations from the assay with the known amino acid compositions of the coated insulin-Fc fusion protein library, one can determine whether particular amino acid mutations or epitopes are responsible for causing none, some, most, or all of the total antibody signal on the assay, indicating no binding, weak binding, or strong binding to various insulin-Fc fusion protein homodimers. The mutations or epitopes responsible for moderate or strong binding are herein referred to as immunogenic “hot spots”.

### Example 14: In Vitro Fc(Gamma) Receptor I Binding Affinity Assay

The binding of insulin-Fc fusion proteins to the Fc(gamma) receptor I at pH 7.4 is conducted using an ELISA assay as follows. Since canine Fc(gamma) receptor I is not commercially available, human Fc(gamma) receptor I (i.e., rhFc(gamma) receptor I) is used as a surrogate mammalian receptor. Insulin-Fc compounds are diluted to 10 μg/mL in sodium bicarbonate buffer at pH 9.6 and coated on Maxisorp (Nunc) microtiter plates overnight at 4° C., after which the microplate strips are washed 5 times with PBST (PBS/0.05% Tween-20) buffer and blocked with Superblock blocking reagent (ThermoFisher). Serial dilutions of biotinylated rhFc(gamma) receptor I (recombinant human Fc(gamma)R—I; R & D Systems) are prepared in PBST/10% Superblock buffer from 6000 ng/mL to 8.2 ng/mL and loaded at 100 μL/well onto the microplate strips coated with insulin-Fc fusion protein. The microtiter plate is incubated for 1 hour at room temperature after which the microplate strips are washed 5 times with PBST and then loaded with 100 μL/well of streptavidin-RP diluted 1:10000 in PBST/10% Superblock buffer. After incubating for 45 min, the microplate strips are washed again 5 times with PBST. TMB is added to reveal the bound Fc(gamma) receptor I proteins and stopped with ELISA stop reagent (Boston Bioproducts). The plate is read in an ELISA plate reader at 450 nm, and the OD values (proportional to the binding of rhFc(gamma) receptor I to insulin-Fc protein) are plotted against log concentrations of rhFc(gamma) receptor I added to each well to generate binding curves using GraphPad Prism software.

### Example 15: In Vitro C1q Binding Affinity Assay

The binding of insulin-Fc fusion proteins to complement component C1q at pH 7.4 is conducted using an ELISA assay as follows using human complement component C1q. Insulin-Fc compounds are diluted to 10 μg/mL in sodium bicarbonate buffer at pH 9.6 and coated on Maxisorp (Nunc) microtiter plates overnight at 4° C., after which the microplate strips are washed 5 times with PBST (PBS/0.05% Tween-20) buffer and blocked with Superblock blocking reagent (ThermoFisher). Serial dilutions of biotinylated complement component C1q (human complement component C1q; Sigma-Aldrich) are prepared in PBST/10% Superblock buffer from 1000 ng/mL to 1.4 ng/mL and loaded at 100 μL/well onto the microplate strips coated with insulin-Fc fusion protein. The microtiter plate is incubated for 1 hour at room temperature after which the microplate strips are washed 5 times with PBST and then loaded with 100 μL/well of streptavidin-HRP diluted 1:12000 in PBST/10% Superblock buffer. After incubating for 45 min, the microplate strips are washed again 5 times with PBST. TMB is added to reveal the bound complement C1q proteins and stopped with ELISA stop reagent (Boston Bioproducts). The plate absorbance is read in an ELISA plate reader at 450 nm (OD450), and the OD values (proportional to the binding of complement component C1q to insulin-Fc protein) are plotted against log concentrations of complement component C1q added to each well to generate binding curves using GraphPad Prism software. For groupings of compounds with somewhat similar curves, the OD450 at one of the higher concentrations, for instance at complement component C1q concentrations of 1000 ng/mL, can be used to identify differences between coated insulin-Fc fusion protein compounds.

### Example 16: Exemplary Insulin-Fc Fusion Protein Domains and Sequences

Exemplary insulin-Fc fusion protein domains and sequences used in the above Examples are shown in Table A and FIG. 2 and FIG. 3.

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprise(s),” “comprising,” “contain(s),” and “containing” are intended to be open and the use thereof permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

1. A fusion protein comprising an insulin polypeptide and an Fc fragment, wherein the fusion protein comprises the sequence:
(SEQ ID NO: 18)
FVNQHLCGSDLVEALALVCGERGFFYTDPTGGGPRRGIVEQCCHSICSLY
QLENYCNGGGGAGGGGDKTHTCPPCPAPELLGGPSVFLFPPKPKDTLMIS
RTPEVTCVVVDVSHEDPEVKFNWYVDGVEVHNAKTKPREEQYSSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPG.

2. A fusion protein comprising an insulin polypeptide and an Fc fragment, wherein the fusion protein comprises the sequence:
(SEQ ID NO: 20)
FVNQHLCGSDLVEALALVCGERGFFYTDPTGGGPRRGIVEQCCHSICSLY
QLENYCNGGGGAGGGGDKTHTCPPCPAPELLGGPSVFLFPPKPKDTLMIS
RTPEVTCVVVDVSHEDPEVKFNWYVDGVEVHNAKTKPREEQYQSTYRVVS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPG

3. A fusion protein comprising an insulin polypeptide, a linker, and an Fc fragment, wherein the insulin polypeptide comprises the sequence:
(SEQ ID NO: 6)
FVNQHLCGSDLVEALALVCGERGFFYTDPTGGGPRRGIVEQCCHSICSLY
QLENYCN
and wherein the fusion protein has a maximum insulin receptor binding IC50 ratio relative to recombinant human insulin (RHI) of less than or equal to 50, less than or equal to 40, less than or equal to 30, or more preferably less than or equal to 20.

4. The fusion protein of claim 3, wherein the Fc fragment of the fusion protein comprises the sequence:
(SEQ ID NO: 32)
DKTHTCPPCPAPELLGGPSVFLFPPKPKDTLMISRTPEVTCVVVDVSHED
PEVKFNWYVDGVEVHNAKTKPREEQYSSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG.

5. The fusion protein of claim 3, wherein the Fc fragment of the fusion protein comprises the sequence:
(SEQ ID NO: 33)
DKTHTCPPCPAPELLGGPSVFLFPPKPKDTLMISRTPEVTCVVVDVSHED
PEVKFNWYVDGVEVHNAKTKPREEQYQSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG.

6. The fusion protein of claim 3, wherein the insulin polypeptide and the Fc fragment are connected by a linker, wherein the linker comprises the sequence:
(SEQ ID NO: 11)
GGGGAGGGG.

7. The fusion protein of claim 3, wherein the fusion protein comprises a dimer of two identical monomers bound together via disulfide bonds, e.g., the fusion protein is a homodimer.

8. The fusion protein of claim 3, wherein the fusion protein comprises domains in the following orientation from N- to C-termini: (N-terminus)—insulin polypeptide—linker—Fc fragment—(C-terminus), and wherein the insulin polypeptide comprises domains in the following orientation from N- to C-termini: (N-terminus)—B-chain—C-peptide—A-chain-(C-terminus).

9. A pharmaceutical composition, said composition comprising a fusion protein of claim 3 dispersed in a pharmaceutically acceptable carrier.

10. A method of inhibiting cancer cell metabolism, growth, and/or proliferation, said method comprising administering an effective amount of a fusion protein of claim 3 to a subject in need thereof.

11. A method of downregulating insulin-like growth factor 1 receptor (IGF1R) in a subject in need thereof, said method comprising administering an effective amount of a fusion protein of claim 3, wherein IGF1R is downregulated in said subject after said administration, wherein said subject has a reduction in tumor volume as compared to an untreated control tumor.

12. The method of claim 10, wherein said fusion protein inhibits tumor growth in said subject after said administration, and wherein said subject has a reduction in tumor volume of at least 30% after said administration as compared to an untreated control tumor.

13. (canceled)

14. The method of claim 10, wherein said fusion protein exhibits an anti-tumor effect on a cancer cell in said subject after said administering, said anti-tumor effect being selected from the group consisting of downregulation of insulin receptor, decreased phosphorylated Akt, and a combination thereof, as compared to an untreated control cancer cell.

15. The method of claim 10, wherein said fusion protein is administered via a route of administration selected from the group consisting of intravenous injection, subcutaneous injection, intratumoral injection, bolus, infusion, and intravenous push, optionally wherein said fusion protein is administered through syringe injection, pump, pen, needle, or indwelling catheter.

16.-17. (canceled)

18. The method of claim 10, wherein said fusion protein is administered to said subject at a dose of from about 150 to about 1,500 micrograms per kilogram of body weight per day.

19. The method of claim 10, wherein said fusion protein is co-administered with a primary or secondary cancer therapy selected from the group consisting of chemotherapy agents, tamoxifen agonists, or antibodies against the IGF1R.

20. The method of claim 10, wherein fusion protein is administered to said subject under fasted conditions.

21. The method of claim 10, wherein said subject has been diagnosed with a cancer selected from the group consisting of breast cancer, colorectal cancer, and melanoma.

22.-33. (canceled)

34. A recombinant cell comprising a nucleic acid encoding the fusion protein of claim 3.

35. A method of preparing a recombinant cell according to claim 34, said method comprising:
transfecting a host cell with a nucleic acid encoding for said fusion protein comprising said insulin polypeptide and said Fc fragment connected by said linker, wherein said fusion protein is expressed in said recombinant cell after said transfecting step.


SEQUENCE LISTING (53 sequences)

SEQ ID NO: 1 - protein, 292 aa, synthetic construct
FVNQHLCGSDLVEALALVCGERGFFYTDPTGGGPRRGIVEQCCHSICSLYQLENYCNGGGGAGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 936 bp, synthetic construct
  source 1..936: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 3 - protein, 51 aa, Homo sapiens
FVNQHLCGSHLVEALYLVCGERGFFYTPKTGIVEQCCTSICSLYQLENYCN

SEQ ID NO: 4 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 30 aa, Homo sapiens
FVNQHLCGSHLVEALYLVCGERGFFYTPKT

SEQ ID NO: 6 - protein, 57 aa, synthetic construct
FVNQHLCGSDLVEALALVCGERGFFYTDPTGGGPRRGIVEQCCHSICSLYQLENYCN

SEQ ID NO: 7 - protein, 30 aa, synthetic construct
FVNQHLCGSDLVEALALVCGERGFFYTDPT

SEQ ID NO: 8 - protein, 6 aa, Synthetic construct
GGGPRR

SEQ ID NO: 9 - protein, 21 aa, Homo sapiens
GIVEQCCTSICSLYQLENYCN

SEQ ID NO: 10 - protein, 21 aa, synthetic construct
GIVEQCCHSICSLYQLENYCN

SEQ ID NO: 11 - protein, 9 aa, synthetic construct
GGGGAGGGG

SEQ ID NO: 12 - protein, 9 aa, synthetic construct
GGGGSGGGG

SEQ ID NO: 13 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 19 aa, synthetic construct
MEWSWVFLFFLSVTTGVHS

SEQ ID NO: 15 - DNA, 57 bp, synthetic construct
  source 1..57: mol_type = other DNA
atggaatggagctgggtctttctcttcttcctgtcagtaacgactggtgtccactcc

SEQ ID NO: 16 - protein, 292 aa, synthetic construct
FVNQHLCGSDLVEALALVCGEEGFFYTDPTGGGPRRGIVEQCCHSICSLYQLENYCNGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - DNA, 936 bp, synthetic construct
  source 1..936: mol_type = other DNA
atggaatggagctgggtctttctcttcttcctgtcagtaacgactggtgtccactccttcgtgaaccagcacctgtgcggctccgacctggtggaagctctggctctcgtgtgcggcgaggagggcttcttctacaccgatcccactggaggcggtccacgcagaggcatcgtggaacagtgctgccactccatctgctccctgtaccagctggaaaactactgcaatggcggaggtggtagcggaggcggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 18 - protein, 292 aa, synthetic construct
FVNQHLCGSDLVEALALVCGERGFFYTDPTGGGPRRGIVEQCCHSICSLYQLENYCNGGGGAGGGGDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - DNA, 936 bp, synthetic construct
  source 1..936: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacagcagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 20 - protein, 292 aa, synthetic construct
FVNQHLCGSDLVEALALVCGERGFFYTDPTGGGPRRGIVEQCCHSICSLYQLENYCNGGGGAGGGGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - DNA, 936 bp, synthetic construct
  source 1..936: mol_type = other DNA
atggaatggagctgggtctttctcttcttcctgtcagtaacgactggtgtccactccttcgtgaaccagcacctgtgcggctccgacctggtggaagctctggctctcgtgtgcggcgagcggggcttcttctacaccgatcccactggaggcggtccacgcagaggcatcgtggaacagtgctgccactccatctgctccctgtaccagctggaaaactactgcaatggcggaggtggtgcaggaggcggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 22 - protein, 285 aa, synthetic construct
FVNQHLCGSDLVEALALVCGEEGFFYTDKGIVEQCCHSICSLYQLENYCNGGGGAGGGGDQTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - DNA, 915 bp, synthetic construct
  source 1..915: mol_type = other DNA
atggaatggagctgggtctttctcttcttcctgtcagtaacgactggtgtccactccttcgtgaaccagcacctgtgcggctccgacctggtggaagctctggctctcgtgtgcggcgaggagggcttcttctacaccgacaagggcatcgtggaacagtgctgccactccatctgctccctgtaccagctggaaaactactgcaatggcggaggtggtgcaggaggcggtggagaccaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 24 - protein, 285 aa, synthetic construct
FVNQHLCGSDLVEALALVCGEAGFFYTDKGIVEQCCHSICSLYQLENYCNGGGGAGGGGDQTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - DNA, 915 bp, synthetic construct
  source 1..915: mol_type = other DNA
atggaatggagctgggtctttctcttcttcctgtcagtaacgactggtgtccactccttcgtgaaccagcacctgtgcggctccgacctggtggaagctctggctctcgtgtgcggcgaggcaggcttcttctacaccgacaagggcatcgtggaacagtgctgccactccatctgctccctgtaccagctggaaaactactgcaatggcggaggtggtgcaggaggcggtggagaccaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 26 - protein, 285 aa, synthetic construct
FVNQHLCGSDLVEALALVCGEEGFFYTDRGIVEQCCHSICSLYQLENYCNGGGGAGGGGDQTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - DNA, 915 bp, synthetic construct
  source 1..915: mol_type = other DNA
atggaatggagctgggtctttctcttcttcctgtcagtaacgactggtgtccactccttcgtgaaccagcacctgtgcggctccgacctggtggaagctctggctctcgtgtgcggcgaggagggcttcttctacaccgaccgaggcatcgtggaacagtgctgccactccatctgctccctgtaccagctggaaaactactgcaatggcggaggtggtgcaggaggcggtggagaccaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 28 - protein, 285 aa, synthetic construct
FVNQHLCGSHLVEALALVCGEEGFFYTDKGIVEQCCHSICSLYQLENYCNGGGGAGGGGDQTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - DNA, 915 bp, synthetic construct
  source 1..915: mol_type = other DNA
atggaatggagctgggtctttctcttcttcctgtcagtaacgactggtgtccactccttcgtgaaccagcacctgtgcggctcccacctggtggaagctctggctctcgtgtgcggcgaggagggcttcttctacaccgacaagggcatcgtggaacagtgctgccactccatctgctccctgtaccagctggaaaactactgcaatggcggaggtggtgcaggaggcggtggagaccaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 30 - protein, 285 aa, synthetic construct
FVNQHLCGSHLVEALALVCGEEGFFYTDRGIVEQCCHSICSLYQLENYCNGGGGAGGGGDQTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - DNA, 915 bp, synthetic construct
  source 1..915: mol_type = other DNA
atggaatggagctgggtctttctcttcttcctgtcagtaacgactggtgtccactccttcgtgaaccagcacctgtgcggctcccacctggtggaagctctggctctcgtgtgcggcgaggagggcttcttctacaccgacaggggcatcgtggaacagtgctgccactccatctgctccctgtaccagctggaaaactactgcaatggcggaggtggtgcaggaggcggtggagaccaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 32 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 226 aa, synthetic construct
DQTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 30 aa, synthetic construct
FVNQHLCGSDLVEALALVCGEEGFFYTDPT

SEQ ID NO: 36 - protein, 29 aa, synthetic construct
FVNQHLCGSDLVEALALVCGEEGFFYTDK

SEQ ID NO: 37 - protein, 29 aa, synthetic construct
FVNQHLCGSDLVEALALVCGEAGFFYTDK

SEQ ID NO: 38 - protein, 29 aa, synthetic construct
FVNQHLCGSDLVEALALVCGEEGFFYTDR

SEQ ID NO: 39 - protein, 29 aa, synthetic construct
FVNQHLCGSHLVEALALVCGEEGFFYTDK

SEQ ID NO: 40 - protein, 29 aa, synthetic construct
FVNQHLCGSHLVEALALVCGEEGFFYTDR

SEQ ID NO: 41 - protein, 57 aa, synthetic construct
FVNQHLCGSDLVEALALVCGEEGFFYTDPTGGGPRRGIVEQCCHSICSLYQLENYCN

SEQ ID NO: 42 - protein, 50 aa, synthetic construct
FVNQHLCGSDLVEALALVCGEEGFFYTDKGIVEQCCHSICSLYQLENYCN

SEQ ID NO: 43 - protein, 50 aa, synthetic construct
FVNQHLCGSDLVEALALVCGEAGFFYTDKGIVEQCCHSICSLYQLENYCN

SEQ ID NO: 44 - protein, 50 aa, synthetic construct
FVNQHLCGSDLVEALALVCGEEGFFYTDRGIVEQCCHSICSLYQLENYCN

SEQ ID NO: 45 - protein, 50 aa, synthetic construct
FVNQHLCGSHLVEALALVCGEEGFFYTDKGIVEQCCHSICSLYQLENYCN

SEQ ID NO: 46 - protein, 50 aa, synthetic construct
FVNQHLCGSHLVEALALVCGEEGFFYTDRGIVEQCCHSICSLYQLENYCN

SEQ ID NO: 47 - protein, 304 aa, synthetic construct
FVNQHLCGSHLVEALELVCGERGFHYGGGGGGSGGGGGIVEQCCTSTCSLDQLENYCGGGGGQGGGGQGGGGQGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 972 bp, synthetic construct
  source 1..972: mol_type = other DNA
atggaatggagctgggtctttctcttcttcctgtcagtaacgactggtgtccactccttcgtgaaccagcacctgtgcggctcccacctggtggaagctctggaactcgtgtgcggcgagcggggcttccactacgggggtggcggaggaggttctggtggcggcggaggcatcgtggaacagtgctgcacctccacctgctccctggaccagctggaaaactactgcggtggcggaggtggtcaaggaggcggtggacagggtggaggtgggcagggaggaggcgggggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttag

SEQ ID NO: 49 - protein, 26 aa, synthetic construct
FVNQHLCGSHLVEALELVCGERGFHY

SEQ ID NO: 50 - protein, 11 aa, synthetic construct
GGGGGGSGGGG

SEQ ID NO: 51 - protein, 20 aa, synthetic construct
GIVEQCCTSTCSLDQLENYC

SEQ ID NO: 52 - protein, 21 aa, synthetic construct
GGGGGQGGGGQGGGGQGGGGG

SEQ ID NO: 53 - protein, 57 aa, synthetic construct
FVNQHLCGSHLVEALELVCGERGFHYGGGGGGSGGGGGIVEQCCTSTCSLDQLENYC